(19) United States
(12) Patent Application Publication
(10) Pub. No.: US 2023/0159945 A1
(43) Pub. Date: May 25, 2023

(54) METHOD

(71) Applicants: R.J. REYNOLDS TOBACCO COMPANY, Winston-Salem, NC (US); BRITISH AMERICAN TOBACCO (INVESTMENTS) LIMITED, London (GB)

(72) Inventors: Darlene Madeline Lawson, Winston-Salem, NC (US); Xingpeng Li, Winston-Salem, NC (US); Matthew Edward Humphry, London (GB); Sara Ben Khaled, London (GB)

(21) Appl. No.: 17/995,675

(22) PCT Filed: Apr. 9, 2021
(86) PCT No.: PCT/EP2021/059302

§ 371 Date: Oct. 6, 2022

Related U.S. Application Data
(60) Provisional application No. 63/007,644, filed on Apr. 9, 2020.

(51) Int. Cl. C12N 15/82; A24B 15/10; A24B 13/00; A24F 40/20

(52) U.S. Cl. CPC C12N 15/8243; C12N 15/8218; A24B 15/10; A24B 13/00; A24F 40/20

(57) ABSTRACT

The present invention provides a method for modulating (e.g. decreasing) the nicotine content of a plant (e.g. a tobacco plant) or part thereof, or tobacco plant cell, the method comprising modifying said plant or cell providing at least one mutation in a Nic3 locus. The present invention provides a method for modulating (e.g. decreasing) the nicotine content of a plant (e.g. a tobacco plant) or part thereof, or tobacco plant cell, the method comprising modifying said plant or cell to modulate the expression or activity of at least one Nic3 gene. The present invention also provides for the use of the Nic3 locus for modulating the alkaloid content of a plant, as well as tobacco cells, plants, plant propagation materials, harvested leaves, processed tobaccos, or delivery systems obtainable in accordance with the invention.

### FIELD OF THE INVENTION

The present invention relates to methods of modulating (e.g. decreasing) the alkaloid content e.g. nicotine content of a tobacco plant or part thereof or tobacco plant cell. The invention also extends to methods of modulating the expression and/or activity of polypeptides encoded by genes which modulate alkaloid content within plants. Alternatively, the invention provides methods of modulating the expression and/or activity of genes which encode polypeptides which modulate alkaloid content (e.g. nicotine content) within plants. The invention also extends to methods of modulating (e.g. decreasing) alkaloid content within plants by introducing mutations to tobacco plants or parts thereof or tobacco plant cells. The invention relates to plants produced by any of the methods herein. The invention also extends to constructs, which can be used to modulate the polypeptides, tobacco plant cells transformed with such constructs, and to transgenic tobacco plants themselves. The invention also relates to the use of harvested leaves from tobacco plants according to the invention having modulating alkaloid content (e.g. nicotine content), and delivery systems (e.g. combustible aerosol provision system, non-combustible aerosol provision system or aerosol-free delivery systems) comprising such leaves or extracts thereof. The invention also relates to the use of tobacco plants according to the invention having low alkaloid content (e.g. nicotine content) in molecular farming.

### BACKGROUND

Alkaloids are a group of naturally occurring compounds which mostly contain basic nitrogen atoms and are produced by a large variety of organisms including bacteria, fungi, plants and animals. Alkaloids may be classified according to the similarity of the carbon skeleton e.g. indole-, isoquinoline- and pyridine-like. Pyridine derivatives are one class of monomeric alkaloids; this class includes simple derivatives of pyridine, polycyclic condensed and noncondensing pyridine derivatives and sesquiterpene pyridine derivatives. Examples are nicotine, nornicotine, anabasine, myosmine and anatabine. Most of the known biological functions of alkaloids are related to protection.

Nicotine occurs naturally in several varieties of plant but is found at the highest level in the tobacco plant. It is produced in wild and cultivated Nicotiana species and it plays an important role in plant defence against herbivores and insects (Voelckel et al., 2001, incorporated herein by reference), accounting for ˜90% of the total alkaloid content. The remaining 10% of the alkaloid pool is mostly constituted by nornicotine, anatabine, myosmine and anabasine.

The regulation of alkaloid content in tobacco is complex. Several factors including genotype, environment, fertilization and agronomic practices (e.g. topping) affect alkaloid levels in tobacco plants.

In the 1930s, certain Cuban cigar tobacco (Nicotiana tabacum) types were identified as having very low alkaloid contents, and this trait was introduced into US tobacco breeding lines (Valleau 1949, incorporated herein by reference). The low-alkaloid trait was subsequently incorporated into the genetic background of cultivar Burley 21 (B21) through multiple generations of backcrosses (Legg et al., 1970, incorporated herein by reference).

Genetic studies using low-alkaloid Burley 21 (LA-B21) suggested that two unlinked loci, initially referred to as locus A and B (Legg et al., 1969, incorporated by reference), but later known as Nic1 and Nic2, contribute to the nicotine levels in tobacco leaf as regulatory loci for nicotine biosynthesis (Legg and Collins 1971, incorporated by reference; Hibi et al., 1994, incorporated by reference). LA B21 was reported to be more susceptible to insect damage, in line with the role of alkaloids in plant defence. It has also been reported that isogenic lines of flue-cured tobacco with low total alkaloids (around 0.2%) have lower yield. By means of haploid doubling of F₁ progeny from the cross between the wild type or high alkaloid B21 (HA-B21, AABB)×LA-B21 (aabb), Collins et al., (1974, incorporated by reference) developed another two isogenic lines (NILs) of B21 with high intermediate alkaloid (HI-B21, AAbb) and low intermediate (LI-B21, aaBB), which were later registered as varieties in 1988 (Nielsen et al., 1988 incorporated by reference). The near isogenic lines (NILs) are referred to herein as Burley 21 (B21, Nic1Nic2), High Intermediate (HI, Nic1nic2), Low Intermediate (LI, nic1Nic2) and Low Alkaloid B21 (LA, nic1nic2) were later registered as varieties in 1988.

Down regulation of nicotine biosynthesis genes in the nic mutants has been confirmed by various research (Hibi et al., 1994; Reed and Jelesko 2004 both incorporated herein by reference) and it is suggested that Nic1 and Nic2 are the regulatory loci that specifically control the expression of nicotine-related structural genes. Subsequent studies have shown that these two loci also control the expression of numerous genes unrelated to nicotine biosynthesis, such as stress response genes (Kidd et al. 2006 incorporated by reference).

Microarray analysis of HA-B21 and LA-B21 led to identification of a cluster of ERF (ethylene response factor) genes regulating nicotine biosynthesis at the Nic2 locus (Shoji et al., 2010, incorporated herein by reference). In the nic2 mutant lines, such as LA-B21 and HI-B21, these ERF genes are deleted altogether. Recent studies have confirmed that the Nic2 region contains a cluster of ERF genes and is located on chromosome 19 (Kajikawa et al. 2017, incorporated herein by reference).

Elucidation of the location of the Nic1 locus in tobacco has proven difficult, due to the complex nature of quantitative traits such as alkaloid levels inhibiting map-based cloning approaches. For the first time Humphry et al., demonstrated in WO2018/237107 (incorporated herein by reference) that a Nic1 locus located on chromosome 7 contains a number of homeologs of the Nic2 ERF genes, and showed that these Nic1 ERF genes function in regulating nicotine levels suggesting that Nic1 may regulate nicotine biosynthesis in a similar manner to Nic2.

Modifying alkaloid content in plants (e.g. tobacco) can have several commercial advantages. For example, decreasing total alkaloid content in plants can increase the value of said plant as a biomass resource. For example, modifying alkaloid content may comprise reducing the alkaloid content e.g. nicotine content of tobacco plants. Tobacco plants and products with reduced nicotine may be desirable in view of the potential regulation of “nicotine ceilings” i.e. average upper limits of nicotine in delivery systems. Alternatively, increasing alkaloid content in plants e.g. tobacco plants, can help to protect plants against insects and herbivores. There remains a need for plants with modulated alkaloid content, for example with modulated nicotine content, with improved commercially desirable traits and methods for making the same.

Tobacco pyridine alkaloids are precursors of tobacco-specific nitrosamines (TSNAs) that form during the post-harvest leaf curing. The four primary TSNAs found in cured tobacco leaves are N′-nitrosonornicotine (NNN), N′nitrosoanatabine (NAT), N′-nitrosoanabasine (NAB) and 4-(methyl nitrosamino)-1-(3-pyridyl)-1-butanone (NNK).

TSNAs form when nitrous oxide species (e.g. NO, NO₂, N₂O₃ and N₂O₄) react with tobacco alkaloids. NAT and NAB are formed via the nitrosation of the secondary alkaloids anatabine and anabasine, respectively. Although early studies claimed that NNN originates from both nicotine and nornicotine, more recent reports have demonstrated that the occurrence of NNN in cured tobacco leaves is correlated with nornicotine content, not nicotine (Bush et al., Rec. Adv. Tob. Sci. 27; 23-46 (2001); Lewis et al., Plant Biotech J. 6: 346-354 (2008) both incorporated herein by reference). Nornicotine is the demethylated derivative of nicotine, the major alkaloid in tobacco accounting for 90% of the total alkaloid content (Saitoh et al., 1985 Phytochemistry, 24 pp. 477-480, incorporated herein by reference). The precursor/product relationship of NNK formation is less clear. Some studies state that NNK is a nitrosation product of nicotine, but due to the slow reaction rate of nicotine nitrosation, it is likely that an oxidized derivative(s) of nicotine, rather than nicotine itself serves as the direct precursor of NNK (Caldwell et al Ann. N.Y. Acad. Sci. 686,213-228 (1993) incorporated herein by reference). Identifying the genes responsible of the production and regulation of the TSNA precursors is of high importance.

Although nornicotine typically accounts for only 2-4% of the total pyridine alkaloid content in tobacco plants, the genetic instability that leads to the spontaneous appearance of high nornicotine-containing converter plants is a chronic problem in delivery systemion. Maintaining low nornicotine levels may prevent the objectionable flavour and aroma associated with this alkaloid, as well as reducing the formation of N-nitrosonornicotine (NNN) in delivery systems, of which nornicotine is the direct precursor.

The gene responsible for the majority of the nicotine to nornicotine conversion is a nicotine demethylase gene CYP82E4, encoding a cytochrome P450 monooxygenase (Siminszky et al., Proc. Natl. Acad. Sci. USA, 102 (2005), pp. 14919-14924; Xu et al., Physiol. Plantarum, 129 (2007), pp. 307-319, both incorporated herein by reference). The nicotine demethylase gene family in tobacco is extensively characterised, but little is known about other cell processes that can influence nornicotine levels.

There still exists a great need to devise methodologies that can further reduce the levels of TSNAs in tobacco plants and products produced from tobacco plants.

As described in the Examples, the inventors sought to investigate genes responsible for alkaloid synthesis, with the aim of modulating alkaloid content in plants, e.g. decreasing nicotine content in tobacco plants.

A flue-cured tobacco variety (FC101) containing nic1 and nic2, was found to have lower than expected nicotine levels, based on these two loci alone. It was hypothesized that a third locus, Nic3, was controlling the reduced nicotine levels in this variety.

Their research prompted the present inventors to create a population segregating for Nic3, generated from a cross between FC101 (nic1 nic2 nic3) and LAFC53 (nic1 nic2 Nic3). The alkaloid content of the resulting F₂ plants was analysed. SNP genotyping was performed on Fes to identify polymorphic markers for further analysis. The inventors developed markers which segregate with the Nic3 locus. New potential regulators of alkaloid synthesis genes were identified.

### SUMMARY OF THE INVENTION

It has been surprisingly found that by modulating the activity or expression of a gene in a Nic3 locus as taught herein, the alkaloid content (e.g. nicotine content) of tobacco plants can be modulated. Thereby delivery systems with modulated alkaloid content and commercially desirable traits sought after by consumers of delivery systems can be produced. In some instances, consumers may desire a product with low levels of alkaloid content e.g. low levels of nicotine content.

The present invention may be particularly useful in the field of plant molecular farming, where plants, or parts thereof or plant cells (such as tobacco and other Nicotiana spp.) are used for the production of proteins, peptides, and metabolites e.g. for the production of therapeutics and pharmaceuticals such as antibiotics, virus like particles, or neutraceuticals or small molecules. Tobacco has been used for the development of an HIV-neutralising antibody in an EU-funded project called PharmPlant and Medicago Inc., Canada have worked on a tobacco-based platform for the production of virus-like particles for flu vaccine manufacture.

In other instances it may be desirable to produce plants with high alkaloid levels e.g. high levels of nicotine content so that nicotine may be purified from the tobacco plant to produce a pure nicotine product for example for use in devices which utilize liquid containing nicotine (e.g. e-cigarettes) or within tobacco heating devices. For example, the production of plants with leaves containing high levels of nicotine could reduce costs of nicotine extraction for the production of e-liquids for e-cigarettes.

The present inventors investigated the regulation of nicotine biosynthesis in tobacco plants. They identified and investigated a new locus, referred to herein as the Nic3 locus. In addition to the regulatory loci Nic1 and Nic2, it is hypothesised that Nic3 controls the expression of nicotine-related structural genes (and possibly other unrelated genes). One aim of the inventors was to provide altered alkaloid content and in particular, reduced nicotine content. Genes were identified in the Nic3 region which unexpectedly modulated alkaloid content in modified tobacco plants compared to their wild-type plant counterparts grown under the same conditions. In particular, the present inventors have identified a Nic3 locus (and Nic3 genes) which are capable of further reducing alkaloid (e.g. nicotine content) and TSNA precursor content in a nic1 nic2 background (i.e. in plants which already have low alkaloid e.g. low nicotine).

The present inventors have surprisingly determined a method for modulating the alkaloid content, e.g. nicotine content, of a tobacco plant or part thereof or plant cell by modulating the activity or expression of a Nic3 gene and/or providing a mutation in a Nic3 locus. Prior to the present invention it had not been known that a third locus—“Nic3” as described herein could be used to modulate alkaloid content alone or in combination with the Nic1 and/or Nic2 loci.

The present inventors have determined that the modulation of a Nic3 locus can reduce the alkaloid content (e.g. nicotine content) and/or TSNA precursor or TSNA content of the modified plant.

In particular, the present inventors have determined that modulation of a Nic1 locus, a Nic2 locus and a Nic3 locus (such as mutation of genes within said loci) provides a plant having surprisingly low alkaloid content (e.g. low nicotine content) and/or low TSNA precursor or low TSNA content.

In one aspect, the present invention provides a method of modulating (e.g. decreasing) the alkaloid content (e.g. nicotine content) of a tobacco plant or a part thereof, or tobacco plant cell, the method comprising modifying said plant or cell by modulating the activity or expression of at least one Nic3 gene from Table 3 or a sequence which has at least 90% identity thereto or a functional variant or functional fragment or orthologue of said gene.

Suitably, in any aspect of the present invention, the activity or expression of at least one Nic3 gene selected from SEQ ID No. 73, SEQ ID No. 76, SEQ ID No. 79, SEQ ID No. 82, SEQ ID No. 85, SEQ ID No. 88, SEQ ID No. 91, SEQ ID No. 94, SEQ ID No. 97, SEQ ID No. 100, SEQ ID No. 103, SEQ ID No. 106, SEQ ID No. 109, SEQ ID No. 112, SEQ ID No. 115, SEQ ID No. 118, SEQ ID No. 121, SEQ ID No. 124, SEQ ID No. 127, SEQ ID No. 130, SEQ ID No. 133, SEQ ID No. 136, SEQ ID No. 139, SEQ ID No. 142, SEQ ID No. 145, SEQ ID No. 148 or SEQ ID No. 151 may be modulated. Suitably, the activity or expression of at least one Nic3 gene selected from SEQ ID No. 73, 118, 124 or 127 may be modulated (e.g. decreased or increased).

Suitably, the activity or expression of at least one gene selected from SEQ ID No. 73, SEQ ID No. 76 or SEQ ID No. 79 may be modulated (e.g. decreased or increased).

In another aspect, the present invention provides a method of modulating (e.g. decreasing) the alkaloid content (e.g. nicotine content) of a tobacco plant or a part thereof, or tobacco plant cell, the method comprising modifying said plant or part thereof, or cell by: introducing at least one mutation to a Nic3 locus (e.g. in a Nic3 gene), and optionally at least one mutation to a Nic1 locus (e.g. in a Nic1 ERF gene) and/or at least one mutation to a Nic2 locus (e.g. in a Nic2 ERF gene). Suitably, in any aspect of the present invention, the Nic3 gene may be selected from SEQ ID No. 73, SEQ ID No. 76, SEQ ID No. 79, SEQ ID No. 82, SEQ ID No. 85, SEQ ID No. 88, SEQ ID No. 91, SEQ ID No. 94, SEQ ID No. 97, SEQ ID No. 100, SEQ ID No. 103, SEQ ID No. 106, SEQ ID No. 109, SEQ ID No. 112, SEQ ID No. 115, SEQ ID No. 118, SEQ ID No. 121, SEQ ID No. 124, SEQ ID No. 127, SEQ ID No. 130, SEQ ID No. 133, SEQ ID No. 136, SEQ ID No. 139, SEQ ID No. 142, SEQ ID No. 145, SEQ ID No. 148 or SEQ ID No. 151.

Suitably, the activity or expression of at least one gene selected from SEQ ID No. 73, 118, 124 or 127 may be modulated (e.g. decreased or increased). Suitably, the activity or expression of at least one gene selected from SEQ ID No. 73, SEQ ID No. 76 or SEQ ID No. 79 may be modulated (e.g. decreased or increased).

In one aspect, the present invention provides a method of modulating (e.g. decreasing) the alkaloid content (e.g. nicotine content) of a tobacco plant or a part thereof, or tobacco plant cell, the method comprising modifying said plant or cell by modulating the activity or expression of:
a) at least one Nic3 gene;
and optionally
b) at least one Nic1 ERF gene; and/or
c) at least one Nic2 ERF gene.

In such methods the activity or expression of at least one Nic3 gene is modified, and optionally, in addition, the activity or expression of at least one Nic1 ERF and/or Nic2 ERF gene may be modified. The term “optionally” as used herein requires that the features that follow are optional only, i.e. may or may not be present. The term “and/or” as used herein allows for the presence of one or both of the features preceding and following that term. In this aspect this therefore covers the alternatives of modifying: i) at least one Nic3 gene; ii) at least one Nic3 gene and at least one Nic1 ERF gene; iii) at least one Nic3 gene and at least one Nic2 ERF gene; and iv) at least one Nic3 gene, at least one Nic1 ERF gene and at least one Nic2 ERF gene. Suitably in option i) there are no modifications in the Nic1 ERF or Nic2 ERF genes. Such options similarly apply to other methods, uses and products described herein in which such modification is contemplated.

In another aspect, the present invention provides a method of modulating (e.g. decreasing) the alkaloid content (e.g. nicotine content) of a tobacco plant or a part thereof, or tobacco plant cell, the method comprising modifying said plant or part thereof, or cell by: introducing at least one mutation to a Nic3 locus (e.g. in a Nic3 gene), and optionally at least one mutation to a Nic1 locus (e.g. in a Nic1 ERF gene) and/or at least one mutation to a Nic2 locus (e.g. in a Nic2 ERF gene). In such methods at least one mutation is introduced to a Nic3 locus, and optionally, in addition, at least one mutation is introduced to a Nic1 locus and/or a Nic2 locus. This therefore covers the alternatives of introducing: i) at least one mutation to a Nic3 locus; ii) at least one mutation to a Nic3 locus and at least one mutation to a Nic1 locus; iii) at least one mutation to a Nic3 locus and at least one mutation to a Nic2 locus; and iv) at least one mutation to a Nic3 locus, at least one mutation to a Nic1 locus and at least one mutation to a Nic2 locus. Suitably in option i) no mutations are introduced to the Nic1 locus or Nic2 locus. Such options similarly apply to other methods, uses and products described herein in which such mutation is contemplated.

In a further aspect, the present invention provides a method of modulating (e.g. decreasing) the content of a tobacco specific nitrosamine (TSNA) precursor in a tobacco plant or plant part thereof, or tobacco plant cell, the method comprising modifying said plant or cell by:
i) modulating the activity or expression of:
   a) at least one Nic3 gene; and optionally
   b) at least one Nic1 ERF gene; and/or
   c) at least one Nic2 ERF gene; or
ii) introducing at least one mutation to a Nic3 locus (e.g. in a Nic3 gene), and optionally at least one mutation to a Nic1 locus (e.g. in a Nic1 ERF gene) and/or at least one mutation to a Nic2 locus (e.g. in a Nic2 ERF gene).

In a further aspect, the present invention provides the use of:
a) at least one Nic3 gene, and optionally at least one Nic1 ERF gene and/or at least one Nic2 ERF gene; or
b) at least one mutation in a Nic3 locus (e.g. in a Nic3 gene), and optionally at least one mutation in a Nic1 locus (e.g. in a Nic1 ERF gene) and/or at least one mutation in a Nic2 locus (e.g. in a Nic2 ERF gene);
for modulating (e.g. decreasing) alkaloid content (e.g. nicotine content) and or TSNA precursor content of a tobacco plant or part thereof or tobacco plant cell.

In another aspect, the present invention provides a method for producing a plant or part thereof, a tobacco plant cell, a tobacco plant propagation material, a tobacco leaf, a cut harvested tobacco leaf, a processed tobacco leaf or a cut and processed tobacco leaf which has modulated (e.g. decreased) alkaloid content (e.g. nicotine content), the method comprising modifying said tobacco plant or part thereof or tobacco cell to:
i) modulate the activity or expression of:
a) at least one Nic3 gene; and optionally
b) at least one Nic1 ERF gene; and/or
c) at least one Nic2 ERF gene; or
ii) introduce at least one mutation in a Nic3 locus (e.g. in a Nic3 gene), and optionally at least one mutation in a Nic1 locus (e.g. in a Nic1 ERF gene) and/or at least one mutation in a Nic2 locus (e.g. in a Nic2 ERF gene).

Suitably, in any aspect of the present invention, the nicotine content may be decreased in comparison to a tobacco plant or part thereof or tobacco cell which has not been modified to introduce at least one mutation to a Nic3 locus, and optionally at least one mutation to a Nic1 locus and/or at least one mutation to a Nic2 locus.

In another aspect, the present invention provides a tobacco plant or part thereof or tobacco cell which has been modified to achieve a reduction in alkaloid content (e.g. nicotine content) in comparison to an unmodified tobacco plant or part thereof or tobacco cell, wherein said modification comprises:
i) modulated activity of or expression of:
a) at least one Nic3 gene; and optionally
b) at least one Nic1 ERF gene; and/or
c) at least one Nic2 ERF gene; or
ii) at least one mutation in a Nic3 locus (e.g. in a Nic3 gene), and optionally at least one mutation in a Nic1 locus (e.g. in a Nic1 gene) and/or at least one mutation in a Nic2 locus (e.g. in a Nic2 ERF gene).

In a further aspect, the present invention provides a tobacco plant propagation material obtainable from a tobacco plant or part thereof or tobacco cell according to the present invention or from a tobacco plant or part thereof or tobacco cell produced by a method according to the present invention.

Suitably, in any aspect of the present invention (such as a method or use according to the present invention, a plant or part thereof or cell according to the present invention, or a plant propagation material according to the present invention):
a) the activity or expression of a Nic3 gene selected from those listed in Table 3 or a sequence which has at least 90% identity thereto or a functional variant or functional fragment or orthologue of said gene may be modulated; or said at least one mutation in the Nic3 locus may be in a Nic3 gene selected from those listed in Table 3 or a sequence which has at least 90% identity thereto or a functional variant or functional fragment or orthologue of said gene:
b) the activity or expression of a Nic1 ERF gene selected from those listed in Table 1 may be modulated; or said at least one mutation in the Nic1 locus may be in a Nic1 ERF gene selected from:
SEQ ID No. 8; or SEQ ID No. 12; or SEQ ID No. 16; or SEQ ID No. 20; or SEQ ID No. 24; or SEQ ID No. 28; or SEQ ID No. 32; or a sequence which has at least 90% identity thereto, or a functional variant or functional fragment or orthologue of said gene; and/or

c) the activity or expression of a Nic2 ERF gene selected from those listed in Table 2 may be modulated; or said at least one mutation in the Nic2 locus is may be a Nic2 ERF gene selected from: SEQ ID No. 69; SEQ ID No. 37; or SEQ ID No. 41; or SEQ ID No. 45; or SEQ ID No. 49; or SEQ ID No. 53; or SEQ ID No. 57; or SEQ ID No. 61; or SEQ ID No. 65; or a sequence which has at least 90% identity thereto or a functional variant or functional fragment or orthologue of said gene. Suitably, a Nic3 gene selected from those listed in Table 3 is selected from SEQ ID No. 73, 118, 124 or 127 or a sequence which has at least 90% identity thereto or a functional variant or functional fragment or orthologue of said gene, and the at least one mutation in the Nic3 locus is in a Nic3 gene selected from SEQ ID No. 73, 118, 124 or 127 or a sequence which has at least 90% identity thereto or a functional variant or functional fragment or orthologue of said gene.

Suitably, in any aspect of the present invention (such as a method or use according to the present invention, a plant or part thereof according to the present invention, or a plant propagation material according to the present invention):
i) the activity or expression of SEQ ID No. 8 may be modulated (e.g. decreased or increased); or said at least one mutation in the Nic1 locus may be in SEQ ID No. 8;
and/or
ii) the activity or expression of SEQ ID No. 69 may be modulated (e.g. decreased or increased); or said at least one mutation in the Nic2 locus may be in SEQ ID No. 69.

Suitably, in any aspect of the present invention (such as a method or use according to the present invention, a plant or part thereof according to the present invention, or a plant propagation material according to the present invention) said at least one mutation in the Nic3 locus may be in a Nic3 gene selected from Table 3, or a sequence which has at least 90% identity thereto or a functional variant or functional fragment or orthologue of said gene.

Suitably, said at least one mutation in the Nic3 gene is selected from:

i) a mutation in the Nic3 gene SEQ ID No. 73 or a sequence which has at least 90% identity thereto or a functional variant or functional fragment or orthologue of said gene that results in a mutation in amino acid residues 74 to 258 or 483-538 of SEQ ID No. 75 or a sequence which has at least 90% identity thereto, or a functional variant or functional fragment or orthologue of said polypeptide;

ii) a mutation in the Nic3 gene SEQ ID No. 118 or a sequence which has at least 90% identity thereto or a functional variant or functional fragment or orthologue of said gene that results in a mutation in amino acid residues 120-584 of SEQ ID No. 120 or a sequence which has at least 90% identity thereto, or a functional variant or functional fragment or orthologue of said polypeptide;

iii) a mutation in the Nic3 gene SEQ ID No. 124 or a sequence which has at least 90% identity thereto or a functional variant or functional fragment or orthologue of said gene that results in a mutation in amino acid residues 166-406 or 483-970 of SEQ ID No. 126 or a sequence which has at least 90% identity thereto, or a functional variant or functional fragment or orthologue of said polypeptide; and

iv) a mutation in the Nic3 gene SEQ ID No. 127 or a sequence which has at least 90% identity thereto or a functional variant or functional fragment or orthologue of said gene that results in a mutation in amino acid residues 171-406 or 509-967 of SEQ ID No. 129 or a sequence which has at least 90% identity thereto, or a functional variant or functional fragment or orthologue of said polypeptide.

In one aspect, the present invention provides the use of a plant or part thereof or plant cell according to the present invention, or of a plant produced by a method according to the present invention to breed a plant.

In one aspect, the present invention provides the use of a plant or part thereof or plant cell according to the present invention, or of a plant produced by a method according to the present invention for production of a product.

In one aspect, the present invention provides the use of a plant or part thereof or plant cell according to the present invention, or of a plant produced by a method according to the present invention to grow a crop.

In one aspect, the present invention provides the use of a plant or part thereof according to the present invention, or of a plant produced by a method according to the present invention to produce a leaf.

In a further aspect, the present invention provides a harvested leaf of a plant according to the present invention, or obtainable from a plant propagated from a propagation material according to the present invention, or obtainable from a plant obtained by a use according to the present invention, or obtainable from a plant produced by a method according to the present invention. The harvested leaf may be green leaf, such as green fresh leaf, or a dried leaf.

Suitably, the harvested leaf according to the present invention may be a cut harvested leaf.

In another aspect, the present invention provides a processed leaf, preferably a processed tobacco leaf, preferably a non-viable processed tobacco leaf:
obtainable (e.g. obtained) from a plant obtainable from a use according to the present invention;
obtainable (e.g. obtained) by processing a plant according to the present invention;
obtainable (e.g. obtained) from a plant propagated from a plant propagation material according to the present invention; or
obtainable (e.g. obtained) by processing a harvested leaf of a plant according to the present invention; or
obtainable (e.g. obtained) from a plant produced by a method according to the present invention.

Suitably, a processed leaf according to the present invention may be processed by curing, fermenting, pasteurising or a combination thereof, preferably wherein the content of one or more TSNAs selected from N′-nitrosonornicotine (NNN), 4-(methylnitrosamino)-1-(3-pyridyl)-1-butanone (NNK), N′-nitrosoanatabine (NAT) and N-nitrosoanabasine (NAB) is decreased, wherein preferably the content of NNN and/or NNK is modulated (e.g. decreased), wherein more preferably the content of NNN is decreased.

Suitably, a processed leaf according to the present invention may be a cut processed leaf.

In a further aspect, the present invention provides cured tobacco material made from a plant or a part thereof:
obtainable (e.g. obtained) from a plant obtainable from a use according to the present invention;
obtainable (e.g. obtained) by processing a plant according to the present invention;
obtainable (e.g. obtained) from a plant propagated from a plant propagation material according to the present invention; or
obtainable (e.g. obtained) by processing a harvested leaf of a plant according to the present invention; or
obtainable from a plant produced by a method according to the present invention.

Suitably, the cured tobacco material, tobacco blend or delivery system, may comprise an average alkaloid level or average nicotine level of about 0.01%, 0.02%, 0.05%, 0.0.75%. 0.1%, 0.2%, 0.3%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3%, 4% or 5% on a dry weight basis. Suitably, the cured tobacco material may comprise an average alkaloid level or average nicotine level of less than 5%, less, than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.075%, less than 0.05%, less than 0.02% or less than 0.01%.

In another aspect, the present invention provides a tobacco blend comprising cured tobacco material according to the present invention.

In a further aspect, the present invention provides a delivery system prepared from:
a tobacco plant according to the present invention, or a part thereof;
a tobacco plant or part thereof propagated from a tobacco plant propagation material according to the present invention;
a harvested leaf of a plant according to the present invention;
a processed leaf according to the present invention;
or
a plant produced by a method according to the present invention.

Suitably, a delivery system according to the present invention may be a combustible smoking article.

Suitably, a delivery system according to the present invention may be a smokeless delivery system.

Suitably, a delivery system according to the present invention may be a non-combustible aerosol provision system such as a tobacco heating device or an aerosol-generating device.

In a further aspect, the present invention provides a combustible smoking article, non-combustible aerosol provisioning system, smokeless delivery system or tobacco heating device comprising a plant or a part thereof according to the present invention or an extract (e.g. a tobacco extract) thereof; or a cured tobacco material according to the present invention; or a tobacco blend according to the present invention.

In another aspect, the present invention provides the use of a nucleotide sequence of a Nic3 locus (e.g. a Nic3 gene from Table 3 or selected from a gene with SEQ ID No. 73, 118, 124 or 127, or a sequence which has at least 90% identity thereto or a functional variant or functional fragment or orthologue of said gene), and optionally a Nic1 locus (e.g. a Nic1 ERF gene) and/or a Nic2 locus (e.g. a Nic2 ERF gene), to select a plant having reduced alkaloid content (e.g. nicotine content) and/or reduced content of tobacco specific nitrosamine (TSNA) or a precursor of a TSNA. Suitably, said nucleotide sequence may comprise a mutation.

In a further aspect, the present invention provides a mutant of a plant carrying at least one heritable mutation in a Nic3 locus (e.g. in a Nic3 gene from Table 3, or selected from a gene with SEQ ID No. 73, 118, 124 or 127 or a sequence which has at least 90% identity thereto or a functional variant or functional fragment or orthologue of said gene), and optionally at least one heritable mutation in a Nic1 locus (e.g. in a Nic1 ERF gene) and/or at least one heritable mutation in a Nic2 locus (e.g. in a Nic2 ERF gene); wherein said heritable mutations decrease the alkaloid content (e.g. nicotine content), and/or decrease the content of a tobacco specific nitrosamine (TSNA) or a precursor of a TSNA in the mutant tobacco plant relative to a comparable plant which does not carry said heritable mutations.

In a further aspect, the present invention provides progeny or seed of a mutant plant which carries the heritable mutation according to the present invention.

In another aspect, the present invention provides a harvested leaf, a processed leaf or cured tobacco material produced from a plant comprising at least one mutation in a Nic3 locus (e.g. in a Nic3 gene from Table 3, or selected from a gene with SEQ ID No. 73, 118, 124 or 127 or a sequence which has at least 90% identity thereto or a functional variant or functional fragment or orthologue of said gene), and optionally at least one mutation in a Nic1 locus (e.g. in a Nic1 ERF gene) and/or at least one mutation in a Nic2 locus (e.g. in a Nic2 ERF gene) and wherein said plant has decreased nicotine content and/or decreased content of a tobacco specific nitrosamine (TSNA) or a precursor of a TSNA relative to a comparable plant which does not carry said mutations in a Nic3 locus, and optionally a Nic1 locus and/or a Nic2 locus.

### BRIEF DESCRIPTION OF THE DRAWINGS

Embodiments of the invention will now be described, by way of example only, with reference to the accompanying drawings, in which:

FIG. 1 shows the nicotine and nornicotine content for FC101 and LAFC53. Asterisks indicate significant difference to FC101 (p-value <0.01)

FIG. 2 shows the nicotine and nornicotine content for an F2 population between FC101 and LAFC53. (A) Nicotine content, (B) Nornicotine content.

FIG. 3 shows nicotine quantitative trait locus (QTL) analysis results for chromosome 5 in an F2 population from FC101×LAFC53.

FIG. 4 shows nicotine and nornicotine content in an F2 population from FC101×LAFC53 segregating for marker Nt2AG2015.

FIGS. 5 to 8 show SEQ ID No. 571 to SEQ ID No. 574 which provide the TRV2 sequences for gene silencing genes with SEQ ID Nos: 73, 118, 124 and 127 in which the gene specific sequences are shown in bold and underlined.

FIG. 9 shows that virus-induced gene silencing of genes in the Nic3 locus results in reduction of nicotine content compared to nic1nic2.

### SEQUENCE LISTING

A summary of sequence identifiers used throughout the subject specification and the corresponding sequence listing is provided wherein:

TABLE 1
The Nic1 locus/Nic1 ERF genes comprise:

| | | Genomic | | | Amino acid |
|---|---|---|---|---|---|
| Identifier | Gene | sequence | cDNA | CDS | sequence |
| Nitab4.5_0003090g0020.1 | ERF17L3ΔN | SEQ ID | SEQ ID | SEQ ID | SEQ ID |
| | | No. 1 | No. 2 | No. 3 | No. 4 |
| Nitab4.5_0003090g0030.1 | ERF199 | SEQ ID | SEQ ID | SEQ ID | SEQ ID |
| | | No. 5 | No. 6 | No. 7 | No. 8 |
| Nitab4.5_0003665g0040.1 | JRE5L2 | SEQ ID | SEQ ID | SEQ ID | SEQ ID |
| | | No. 9 | No. 10 | No. 11 | No. 12 |
| Nitab4.5_0004620g0010.1 | ERF210 | SEQ ID | SEQ ID | SEQ ID | SEQ ID |
| | | No. 13 | No. 14 | No. 15 | No. 16 |
| Nitab4.5_0004620g0030.1 | ERF91 | SEQ ID | SEQ ID | SEQ ID | SEQ ID |
| | | No. 17 | No. 18 | No. 19 | No. 20 |
| Nitab4.5_0004620g0080.1 | ERF29 | SEQ ID | SEQ ID | SEQ ID | SEQ ID |
| | | No. 21 | No. 22 | No. 23 | No. 24 |
| Nitab4.5_0004620g0090.3 | ERF130 | SEQ ID | SEQ ID | SEQ ID | SEQ ID |
| | | No. 25 | No. 26 | No. 27 | No. 28 |
| Nitab4.5_0004620g0095.1 | ERF16 | SEQ ID | SEQ ID | SEQ ID | SEQ ID |
| | | No. 29 | No. 30 | No. 31 | No. 32 |
| Nitab4.5_0006382g0040.1 | ERF110 | SEQ ID | SEQ ID | SEQ ID | SEQ ID |
| | | No. 33 | No. 34 | No. 35 | No. 36 |

TABLE 2
The Nic 2 locus/Nic2 ERFs comprise:

| | | Genomic | | | Amino acid |
|---|---|---|---|---|---|
| Identifier | Gene | sequence | cDNA | CDS | sequence |
| Nitab4.5_0002924g0010.1 | ERF17LI | SEQ ID | SEQ ID | SEQ ID | SEQ ID |
| | | No. 37 | No. 38 | No. 39 | No. 40 |
| Nitab4.5_0002924g0020.2 | ERF179 | SEQ ID | SEQ ID | SEQ ID | SEQ ID |
| | | No. 41 | No. 42 | No. 43 | No. 44 |
| Nitab4.5_0002924g0040.2 | ERF17 | SEQ ID | SEQ ID | SEQ ID | SEQ ID |
| | | No. 45 | No. 46 | No. 47 | No. 48 |
| Nitab4.5_0002924g0045.1 | ERF168 | SEQ ID | SEQ ID | SEQ ID | SEQ ID |
| | | No. 49 | No. 50 | No. 51 | No. 52 |
| Nitab4.5_0002924g0050.2 | ERF115 | SEQ ID | SEQ ID | SEQ ID | SEQ ID |
| | | No. 53 | No. 54 | No. 55 | No. 56 |
| Nitab4.5_0006499g0010.1 | ERF104 | SEQ ID | SEQ ID | SEQ ID | SEQ ID |
| | | No. 57 | No. 58 | No. 59 | No. 60 |
| Nitab4.5_0006499g0020.2 | ERF221 | SEQ ID | SEQ ID | SEQ ID | SEQ ID |
| | | No. 61 | No. 62 | No. 63 | No. 64 |
| Nitab4.5_0012667g0020.2 | ERF91L1 | SEQ ID | SEQ ID | SEQ ID | SEQ ID |
| | | No. 65 | No. 66 | No. 67 | No. 68 |
| Nitab4.5_0015055g0010.2 | ERF189 | SEQ ID | SEQ ID | SEQ ID | SEQ ID |
| | | No. 69 | No. 70 | No. 71 | No. 72 |

TABLE 3
The Nic3 locus/Nic3 genes sequences comprise:

| | | Genomic | | Amino acid |
|---|---|---|---|---|
| Identifier | Predicted function | sequence | CDS | sequence |
| Nitab4.5_0002539g0040.2 | Myc-type, basic helix-loop-helix (bHLH) | SEQ ID | SEQ ID | SEQ ID |
| | domain, Transcription factor MYC/MYB N- | No. 73 | No. 74 | No. 75 |
| | terminal (MYC2a) | | | |
| Nitab4.5_0002683g0020.2 | ERF | SEQ ID | SEQ ID | SEQ ID |
| | | No. 76 | No. 77 | No. 78 |
| Nitab4.5_0002778g0050.2 | Plastid-lipid associated protein PAP/fibrillin | SEQ ID | SEQ ID | SEQ ID |
| | family protein | No. 79 | No. 80 | No. 81 |
| Nitab4.5_0004551g0030.2 | Myb TF | SEQ ID | SEQ ID | SEQ ID |
| | | No. 82 | No. 83 | No. 84 |
| Nitab4.5_0000535g0120.2 | NAC domain protein: involved in plant | SEQ ID | SEQ ID | SEQ ID |
| | hormonal control and defence. DNA-binding | No. 85 | No. 86 | No. 87 |
| | domain (DBD) and a dimerization domain | | | |
| Nitab4.5_0005226g0010.2 | Cytochrome P450 | SEQ ID | SEQ ID | SEQ ID |
| | | No. 88 | No. 89 | No. 90 |
| Nitab4.5_0009200g0010.2 | Cytochrome P450 | SEQ ID | SEQ ID | SEQ ID |
| | | No. 91 | No. 92 | No. 93 |
| Nitab4.5_0004528g0040.2 | BZIP transcription factor family protein | SEQ ID | SEQ ID | SEQ ID |
| | | No. 94 | No. 95 | No. 96 |
| Nitab4.5_0000556g0220.2 | Myb family transcription factor family | SEQ ID | SEQ ID | SEQ ID |
| | protein | No. 97 | No. 98 | No. 99 |
| Nitab4.5_0006594g0090.2 | Myb Transcription factor | SEQ ID | SEQ ID | SEQ ID |
| | | No. 100 | No. 101 | No. 102 |
| Nitab4.5_0006594g0030.2 | GATA transcription factor | SEQ ID | SEQ ID | SEQ ID |
| | | No. 103 | No. 104 | No. 105 |
| Nitab4.5_0006594g0020.2 | Transferase | SEQ ID | SEQ ID | SEQ ID |
| | | No. 106 | No. 107 | No. 108 |
| Nitab4.5_0006594g0050.2 | Nuclear RNA binding protein-like | SEQ ID | SEQ ID | SEQ ID |
| | | No. 109 | No. 110 | No. 111 |
| Nitab4.5_0002683g0040.2 | NB-ARC domain, Disease resistance protein | SEQ ID | SEQ ID | SEQ ID |
| | | No. 112 | No. 113 | No. 114 |
| Nitab4.5_0002683g0070.2/ | Aminotransferase | SEQ ID | SEQ ID | SEQ ID |
| Nitab05g026600.2.2 | | No. 115 | No. 116 | No. 117 |
| Nitab4.5_0002683g0080.2 | LRR, Disease resistance protein | SEQ ID | SEQ ID | SEQ ID |
| | | No. 118 | No. 119 | No. 120 |
| Nitab4.5_0006415g0030.2 | LRR, Disease resistance protein | SEQ ID | SEQ ID | SEQ ID |
| | | No. 121 | No. 122 | No. 123 |
| Nitab4.5_0005412g0010.2 | NB-ARC, Disease resistance protein | SEQ ID | SEQ ID | SEQ ID |
| | | No. 124 | No. 125 | No. 126 |
| Nitab4.5_0005412g0020.2 | NB-ARC, Disease resistance protein | SEQ ID | SEQ ID | SEQ ID |
| | | No. 127 | No. 128 | No. 129 |
| Nitab4.5_0000712g0030.2 | Transcriptional regulation, Homeobox- | SEQ ID | SEQ ID | SEQ ID |
| | leucine zipper family protein | No. 130 | No. 131 | No. 132 |
| Nitab4.5_0000712g0010.2 | Myb family transcription factor family | SEQ ID | SEQ ID | SEQ ID |
| | protein | No. 133 | No. 134 | No. 135 |
| Nitab4.5_0000712g0020.2 | Myb | SEQ ID | SEQ ID | SEQ ID |
| | | No. 136 | No. 137 | No. 138 |
| Nitab4.5_0000712g0040.2 | Myb family transcription factor family | SEQ ID | SEQ ID | SEQ ID |
| | protein | No. 139 | No. 140 | No. 141 |
| Nitab4.5_0022887g0010.2 | Metabolite exporter, WAT1-related protein | SEQ ID | SEQ ID | SEQ ID |
| | | No. 142 | No. 143 | No. 144 |
| Nitab4.5_0000712g0270.2 | Nitrate transporter | SEQ ID | SEQ ID | SEQ ID |
| | | No. 145 | No. 146 | No. 147 |
| Nitab4.5_0008372g0010.2 | Nitrate transporter | SEQ ID | SEQ ID | SEQ ID |
| | | No. 148 | No. 149 | No. 150 |
| Nitab4.5_0012102g0010.2 | Nitrate transporter | SEQ ID | SEQ ID | SEQ ID |
| | | No. 151 | No. 152 | No. 153 |
| Nitab4.5_0007629g0040.2 | Arf gtpase-activating protein | SEQ ID | SEQ ID | SEQ ID |
| | | No. 154 | No. 155 | No. 156 |
| Nitab4.5_0007629g0030.2 | Ubiquitin carboxyl-terminal hydrolase-like | SEQ ID | SEQ ID | SEQ ID |
| | protein | No. 157 | No. 158 | No. 159 |
| Nitab4.5_0008566g0040.2 | Ankyrin repeat-containing protein | SEQ ID | SEQ ID | SEQ ID |
| | | No. 160 | No. 161 | No. 162 |
| Nitab4.5_0002539g0020.2 | CAAX protease self-immunity protein | SEQ ID | SEQ ID | SEQ ID |
| | | No. 163 | No. 164 | No. 165 |
| Nitab4.5_0008566g0030.2 | MLH1 | SEQ ID | SEQ ID | SEQ ID |
| | | No. 166 | No. 167 | No. 168 |
| Nitab4.5_0008650g0030.2 | AT hook motif DNA-binding family protein | SEQ ID | SEQ ID | SEQ ID |
| | | No. 169 | No. 170 | No. 171 |
| Nitab4.5_0008566g0080.2 | PLATZ transcription factor family protein | SEQ ID | SEQ ID | SEQ ID |
| | | No. 172 | No. 173 | No. 174 |
| Nitab4.5_0008650g0010.2 | AT hook motif DNA-binding family protein | SEQ ID | SEQ ID | SEQ ID |
| | | No. 175 | No. 176 | No. 177 |
| Nitab05g026010.1.2 | F-box protein interaction domain protein | SEQ ID | SEQ ID | SEQ ID |
| | | No. 178 | No. 179 | No. 180 |
| Nitab4.5_0008650g0020.2 | AP-2 complex subunit mu | SEQ ID | SEQ ID | SEQ ID |
| | | No. 181 | No. 182 | No. 183 |
| Nitab4.5_0004551g0020.2 | Zinc finger, RING-type domain | SEQ ID | SEQ ID | SEQ ID |
| | | No. 184 | No. 185 | No. 186 |
| Nitab4.5_0000535g0050.2 | Leucine-rich repeat-containing protein | SEQ ID | SEQ ID | SEQ ID |
| | | No. 187 | No. 188 | No. 189 |
| Nitab4.5_0004551g0010.2 | Cinnamoyl-CoA reductase | SEQ ID | SEQ ID | SEQ ID |
| | | No. 190 | No. 191 | No. 192 |
| Nitab4.5_0000535g0020.2 | RING/U-box superfamily protein isoform 1 | SEQ ID | SEQ ID | SEQ ID |
| | | No. 193 | No. 194 | No. 195 |
| Nitab4.5_0000535g0030.2 | aluminum activated malate transporter | SEQ ID | SEQ ID | SEQ ID |
| | family protein | No. 196 | No. 197 | No. 198 |
| Nitab4.5_0000535g0010.2 | ABC transporter family protein | SEQ ID | SEQ ID | SEQ ID |
| | | No. 199 | No. 200 | No. 201 |
| Nitab4.5_0010737g0010.2 | Inositol-tetrakisphosphate 1-kinase | SEQ ID | SEQ ID | SEQ ID |
| | | No. 202 | No. 203 | No. 204 |
| Nitab4.5_0000535g0040.2 | Protein phosphatase 2c | SEQ ID | SEQ ID | SEQ ID |
| | | No. 205 | No. 206 | No. 207 |
| Nitab4.5_0010978g0020.2 | E3 ubiquitin-protein ligase RNF14 | SEQ ID | SEQ ID | SEQ ID |
| | | No. 208 | No. 209 | No. 210 |
| Nitab4.5_0000535g0100.2 | Phosphatase 2C family protein | SEQ ID | SEQ ID | SEQ ID |
| | | No. 211 | No. 212 | No. 213 |
| Nitab4.5_0010978g0010.2 | Pentatricopeptide repeat protein | SEQ ID | SEQ ID | SEQ ID |
| | | No. 214 | No. 215 | No. 216 |
| Nitab4.5_0000535g0110.2 | Protein phosphatase 2c | SEQ ID | SEQ ID | SEQ ID |
| | | No. 217 | No. 218 | No. 219 |
| Nitab4.5_0002778g0010.2 | Heat shock transcription factor | SEQ ID | SEQ ID | SEQ ID |
| | | No. 220 | No. 221 | No. 222 |
| Nitab4.5_0002778g0020.2 | Pre-mRNA-splicing factor CWC22 | SEQ ID | SEQ ID | SEQ ID |
| | | No. 223 | No. 224 | No. 225 |
| Nitab4.5_0002778g0030.2 | Syntaxin-like protein | SEQ ID | SEQ ID | SEQ ID |
| | | No. 226 | No. 227 | No. 228 |
| Nitab4.5_0006398g0040.2 | Glycine cleavage system h protein | SEQ ID | SEQ ID | SEQ ID |
| | | No. 229 | No. 230 | No. 231 |
| Nitab05g025900.1.2 | Replication factor-A carboxy- | SEQ ID | SEQ ID | SEQ ID |
| | terminal domain protein | No. 232 | No. 233 | No. 234 |
| Nitab4.5_0006398g0030.2 | Peptide transporter | SEQ ID | SEQ ID | SEQ ID |
| | | No. 235 | No. 236 | No. 237 |
| Nitab4.5_0002778g0070.2 | Dolichol phosphate-mannose | SEQ ID | SEQ ID | SEQ ID |
| | biosynthesis regulatory protein | No. 238 | No. 239 | No. 240 |
| Nitab4.5_0006398g0030.1/ | Peptide transporter | SEQ ID | SEQ ID | SEQ ID |
| Nitab05g026330.2.2 | | No. 241 | No. 242 | No. 243 |
| Nitab4.5_0006398g0010.2 | Type 1 inositol-1 | SEQ ID | SEQ ID | SEQ ID |
| | | No. 244 | No. 245 | No. 246 |
| Nitab4.5_0002778g0040.2 | GRIP/coiled-coil protein, | SEQ ID | SEQ ID | SEQ ID |
| | putative (DUF1664) | No. 247 | No. 248 | No. 249 |
| Nitab4.5_0010461g0020.2 | Expansin-like protein | SEQ ID | SEQ ID | SEQ ID |
| | | No. 250 | No. 251 | No. 252 |
| Nitab4.5_0002778g0080.2 | Lysine-specific demethylase | SEQ ID | SEQ ID | SEQ ID |
| | | No. 253 | No. 254 | No. 255 |
| Nitab4.5_0010461g0010.2 | Haloacid dehalogenase-like | SEQ ID | SEQ ID | SEQ ID |
| | hydrolase family protein | No. 256 | No. 257 | No. 258 |
| Nitab4.5_0005487g0030.2/ | Regulator of nonsense | SEQ ID | SEQ ID | SEQ ID |
| Nitab05g026390.1.2 | transcripts 1-like protein | No. 259 | No. 260 | No. 261 |
| Nitab4.5_0008709g0020.2 | Regulator of nonsense | SEQ ID | SEQ ID | SEQ ID |
| | transcripts 1 | No. 262 | No. 263 | No. 264 |
| Nitab4.5_0008709g0030.2 | SBP (S-ribonuclease binding | SEQ ID | SEQ ID | SEQ ID |
| | protein) family protein | No. 265 | No. 266 | No. 267 |
| Nitab4.5_0008709g0010.2 | Beta-amylase | SEQ ID | SEQ ID | SEQ ID |
| | | No. 268 | No. 269 | No. 270 |
| Nitab4.5_0008425g0020.2 | 60S ribosomal protein L37a | SEQ ID | SEQ ID | SEQ ID |
| | | No. 271 | No. 272 | No. 273 |
| Nitab4.5_0004528g0020.2 | Cellulose synthase | SEQ ID | SEQ ID | SEQ ID |
| | | No. 274 | No. 275 | No. 276 |
| Nitab4.5_0008425g0040.2 | Calcineurin B-like protein | SEQ ID | SEQ ID | SEQ ID |
| | | No. 277 | No. 278 | No. 279 |
| Nitab4.5_0004528g0030.2 | Dolichyl-diphosphooligosaccharide-protein | SEQ ID | SEQ ID | SEQ ID |
| | glycosyltransferase subunit DAD1 | No. 280 | No. 281 | No. 282 |
| Nitab4.5_0008425g0010.2 | AUGMIN subunit 2 | SEQ ID | SEQ ID | SEQ ID |
| | | No. 283 | No. 284 | No. 285 |
| Nitab4.5_0004528g0010.2 | Biotin/lipoate A/B protein ligase family | SEQ ID | SEQ ID | SEQ ID |
| | | No. 286 | No. 287 | No. 288 |
| Nitab4.5_0008425g0030.2 | Protein kinase | SEQ ID | SEQ ID | SEQ ID |
| | | No. 289 | No. 290 | No. 291 |
| Nitab05g026480.2.2 | Bifunctional inhibitor/lipid-transfer | SEQ ID | SEQ ID | SEQ ID |
| | protein/seed storage 2S albumin superfamily | No. 292 | No. 293 | No. 294 |
| | protein | | | |
| Nitab4.5_0000556g0260.2 | Poly [ADP-ribose] polymerase | SEQ ID | SEQ ID | SEQ ID |
| | | No. 295 | No. 296 | No. 297 |

SEQ ID No. 298 corresponds to marker Nt1AG1750.

SEQ ID No. 299 corresponds to marker Nt1AC2307.

SEQ ID No. 300 is a forward primer for SNP3.

SEQ ID No. 301 is a reverse primer for SNP3.

SEQ ID No. 302 is a forward primer for SNP5.

SEQ ID No. 303 is a reverse primer for SNP5.

SEQ ID No. 304 is a forward primer for SNP15.

SEQ ID No. 305 is a reverse primer for SNP15.

SEQ ID No. 306 is a forward primer for SNP18.

SEQ ID No. 307 is a reverse primer for SNP18.

SEQ ID No. 308 is a forward primer for SNP19.

SEQ ID No. 309 is a reverse primer for SNP19.

SEQ ID No. 310 corresponds to marker Nt2AG2015.

SEQ ID No. 311 corresponds to marker Nt1AG1750.

SEQ ID No. 312 corresponds to marker Nt1AC2307.

SEQ ID Nos. 313-569 are the sequences of the SNPs associated with the QTL identified in the Examples.

SEQ ID No. 570 is the TRV RNA1 used in Example 7.

SEQ ID No. 571-574 are the TRV RNA2 sequences used in Example 7.

Some sequences disclosed herein contain “N” in nucleotide sequences. “N” can be any nucleotide or a deletion or insertion of one or more nucleotides. For example, in some cases a string of “N”s are shown. The number of “N”s does not necessarily correlate with the actual number of nucleotides at that position. There may be more or fewer nucleotides than shown as “N” in the sequence.

### DETAILED DESCRIPTION

For the first time the present inventors have identified the Nic3 locus, which regulates nicotine biosynthesis in tobacco.

By modulating the activity or expression of at least one Nic3 gene in a plant (e.g. a tobacco plant) or part thereof or tobacco cell, the alkaloid and/or TSNA content of the plant may be modulated (e.g. reduced). The alkaloid (e.g. nicotine) content of a plant or part thereof of plant cell may be modulated (e.g. reduced) by introducing a mutation to the Nic3 locus.

As used herein, “Nic3” locus refers to any chromosomal position or location within or closely linked to the Nic3 region.

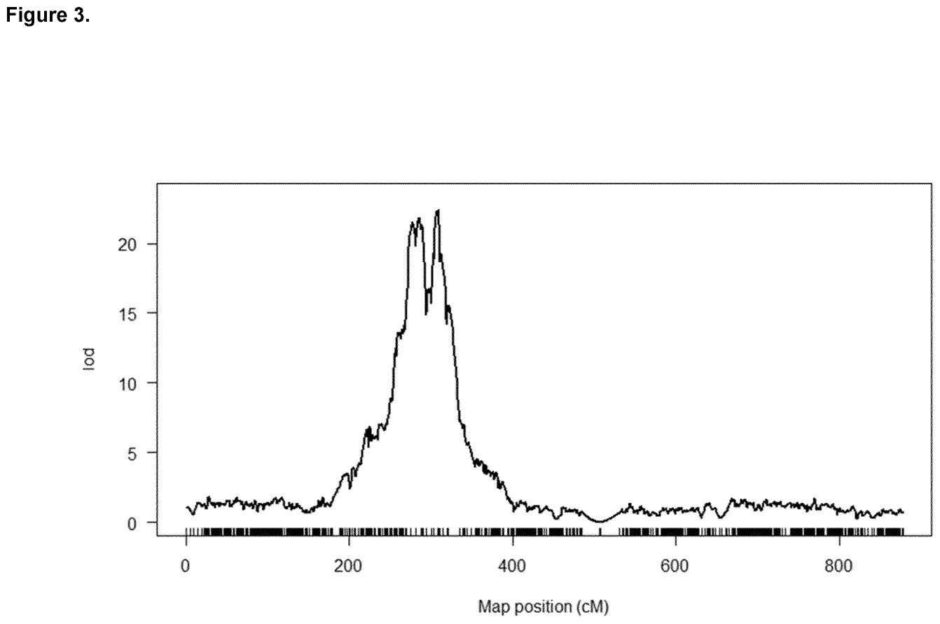

“Nic3 region” refers to a chromosomal segment subtended by the markers Nt1AG1750 (SEQ ID No. 298) and Nt1AC2307 (SEQ ID No. 299), corresponding to 206 cM to 398 cM shown in FIG. 3, and having allele(s) associated with a low alkaloid (low-nicotine) trait.

A “Nic3 mutation” refers to a mutation in a Nic3 locus.

As used herein, a “Nic3 gene” refers to a gene at or near a Nic3 locus and includes for example, the genes listed in Table 3; or a sequence which has at least 80% identity thereto (at least 85%, at least 90%, at least 95%, at least 97%, at least 98%, at least 99% identity thereto), or a functional variant or functional fragment or orthologue of said gene.

Suitably, a Nic3 gene may be selected from: SEQ ID No. 73, SEQ ID No. 76, SEQ ID No. 79, SEQ ID No. 82, SEQ ID No. 85, SEQ ID No. 88, SEQ ID No. 91, SEQ ID No. 94, SEQ ID No. 97, SEQ ID No. 100, SEQ ID No. 103, SEQ ID No. 106, SEQ ID No. 109, SEQ ID No. 112, SEQ ID No. 115, SEQ ID No. 118, SEQ ID No. 121, SEQ ID No. 124, SEQ ID No. 127, SEQ ID No. 130, SEQ ID No. 133, SEQ ID No. 136, SEQ ID No. 139, SEQ ID No. 142, SEQ ID No. 145, SEQ ID No. 148 or SEQ ID No. 151; or a sequence which has at least 80% identity thereto (at least 85%, at least 90%, at least 95%, at least 97%, at least 98%, at least 99% identity thereto), or a functional variant or functional fragment or orthologue of said gene.

Suitably, the Nic3 gene may selected from SEQ ID No. 73, SEQ ID No. 76 or SEQ ID No. 79; or a sequence which has at least 80% identity thereto (at least 85%, at least 90%, at least 95%, at least 97%, at least 98%, at least 99% identity thereto), or a functional variant or functional fragment or orthologue of said gene. Suitably the Nic3 gene may be selected from SEQ ID No. 73, SEQ ID No. 118, SEQ ID No. 124 or SEQ ID No. 127; or a sequence which has at least 80% identity thereto (at least 85%, at least 90%, at least 95%, at least 97%, at least 98%, at least 99% identity thereto), or a functional variant or functional fragment or orthologue of said gene.

In another aspect, a Nic3 locus comprises a sequence or a chromosomal segment within 50, 100, 200, 300, 400, 500, 6000, 700, 800, 900, 1000, 1500, 2000, 3000, 4000, 5000, 6000, 7000, 8000, 9000, 10000, 15000, 20000, 30000 40000, 50000, 60000, 70000 nucleotides of a sequence selected from the group consisting of SEQ ID No. 73, SEQ ID No. 76, SEQ ID No. 79, SEQ ID No. 82, SEQ ID No. 85, SEQ ID No. 88, SEQ ID No. 91, SEQ ID No. 94, SEQ ID No. 97, SEQ ID No. 100, SEQ ID No. 103, SEQ ID No. 106, SEQ ID No. 109, SEQ ID No. 112, SEQ ID No. 115, SEQ ID No. 118, SEQ ID No. 121, SEQ ID No. 124, SEQ ID No. 127, SEQ ID No. 130, SEQ ID No. 133, SEQ ID No. 136, SEQ ID No. 139, SEQ ID No. 142, SEQ ID No. 145, SEQ ID No. 148 and SEQ ID No. 151 (suitably from the group consisting of SEQ ID No. 73, SEQ ID No. 118, SEQ ID No. 124 and SEQ ID No. 127).

In one aspect, the Nic3 gene is SEQ ID No. 73, SEQ ID No. 118, SEQ ID No. 124 or SEQ ID No. 127; or a sequence which has at least 80% identity thereto (at least 85%, at least 90%, at least 95%, at least 97%, at least 98%, at least 99% identity thereto), or a functional variant or functional fragment or orthologue of said gene.

Suitably, the Nic3 gene may encode a polypeptide comprising an amino acid sequence set forth in Table 3; or a sequence which has at least 80% identity thereto (at least 85%, at least 90%, at least 95%, at least 97%, at least 98%, at least 99% identity thereto), or a functional variant or functional fragment or orthologue of said polypeptide.

Suitably, the Nic3 gene may encode a polypeptide selected from: SEQ ID No. 75, SEQ ID No. 78, SEQ ID No. 81, SEQ ID No. 84, SEQ ID No. 87, SEQ ID No. 90, SEQ ID No. 93, SEQ ID No. 96, SEQ ID No. 99, SEQ ID No. 102, SEQ ID No. 105, SEQ ID No. 108, SEQ ID No. 111, SEQ ID No. 114, SEQ ID No. 117, SEQ ID No. 120, SEQ ID No. 123, SEQ ID No. 126, SEQ ID No. 129, SEQ ID No. 132, SEQ ID No. 135, SEQ ID No. 138, SEQ ID No. 141, SEQ ID No. 144, SEQ ID No. 147, SEQ ID No. 150 or SEQ ID No. 153; or a sequence which has at least 80% identity thereto (at least 85%, at least 90%, at least 95%, at least 97%, at least 98%, at least 99% identity thereto), or a functional variant or functional fragment or orthologue of said polypeptide.

Suitably, the Nic3 gene may encode a polypeptide selected from: SEQ ID No. 75, SEQ ID No. 78 or SEQ ID No. 81; or a sequence which has at least 80% identity thereto (at least 85%, at least 90%, at least 95%, at least 97%, at least 98%, at least 99% identity thereto), or a functional variant or functional fragment or orthologue of said polypeptide.

In one aspect, the Nic3 gene encodes a polypeptide comprising the amino acid sequence SEQ ID No. 75, SEQ ID No. 120, SEQ ID No. 126 or SEQ ID No. 129; or a sequence which has at least 80% identity thereto (at least 85%, at least 90%, at least 95%, at least 97%, at least 98%, at least 99% identity thereto), or a functional variant or functional fragment or orthologue of said polypeptide.

In one aspect a Nic3 locus comprises one or more sequences selected from Table 3.

In one aspect a Nic3 locus comprises one or more sequences selected from the group consisting of: SEQ ID No. 73, SEQ ID No. 76, SEQ ID No. 79, SEQ ID No. 82, SEQ ID No. 85, SEQ ID No. 88, SEQ ID No. 91, SEQ ID No. 94, SEQ ID No. 97, SEQ ID No. 100, SEQ ID No. 103, SEQ ID No. 106, SEQ ID No. 109, SEQ ID No. 112, SEQ ID No. 115, SEQ ID No. 118, SEQ ID No. 121, SEQ ID No. 124, SEQ ID No. 127, SEQ ID No. 130, SEQ ID No. 133, SEQ ID No. 136, SEQ ID No. 139, SEQ ID No. 142, SEQ ID No. 145, SEQ ID No. 148 or SEQ ID No. 151, or a sequence which has at least 80% identity thereto (at least 85%, at least 90%, at least 95%, at least 97%, at least 98%, at least 99% identity thereto), or a functional variant or functional fragment or orthologue of said gene.

Suitably, a Nic3 locus may comprise one or more sequences selected from the group consisting of SEQ ID No. 73, SEQ ID No. 76 and SEQ ID No. 79 or a sequence which has at least 80% identity thereto (at least 85%, at least 90%, at least 95%, at least 97%, at least 98%, at least 99% identity thereto), or a functional variant or functional fragment or orthologue of said gene.

In one aspect, a Nic3 locus comprises at least one or more of SEQ ID No. 73, SEQ ID No. 118, SEQ ID No. 124 or SEQ ID No. 127 or a sequence which has at least 80% identity thereto (at least 85%, at least 90%, at least 95%, at least 97%, at least 98%, at least 99% identity thereto), or a functional variant or functional fragment or orthologue of said gene.

As used herein “closely linked to” or “associated with” means that the marker or locus is within about 20 cM, 1 ocM, 5 cM, 1 cM, 0.5 cM or less than 0.5 cM of another marker or locus. For example, 10 cM means that the recombination between the marker and the locus with a frequency of equal to or less than about 10%.

As used herein, centimorgan (cM) refers to a unit of measure of recombination frequency. A cM is equivalent to a 1% chance that a marker at one genetic locus will be separated from a marker at a second locus due to crossing over in a single generation. Methods for calculating genetic distances from recombination values using the Kosambi function are known in the art, for example in Kosambi, (Annals of Eugenics, 12:172-175 (1944), which is incorporated herein by reference). As used herein, “Nic1” locus refers to any chromosomal position or location within or closely linked to the Nic1 region.

“Nic1 region” refers to a chromosomal segment as disclosed in WO2018/237107 (incorporated herein by reference) for example, a chromosomal segment subtended by the markers SNP3 and SNP5 Nt1AB6591 and Nt1AA9777), and having allele(s) associated with a low alkaloid (low-nicotine) trait. A forward primer for SNP3 is SEQ ID No. 300; a reverse primer for SNP3 is SEQ ID No. 301. A forward primer for SNP5 is SEQ ID No. 302; a reverse primer for SNP5 is SEQ ID No. 303.

A “Nic1 mutation” refers to a mutation in a Nic1 locus.

In one aspect a Nic1 locus comprises one or more sequences selected from Table 1.

In one aspect a Nic1 locus comprises one or more sequences selected from the group consisting of: SEQ ID No. 5, SEQ ID No. 1, SEQ ID No. 9, SEQ ID No. 13, SEQ ID No. 17, SEQ ID No. 21, SEQ ID No. 25, SEQ ID No. 29, or SEQ ID No. 33, or a sequence which has at least 80% identity thereto (at least 85%, at least 90%, at least 95%, at least 97%, at least 98%, at least 99% identity thereto), or a functional variant or functional fragment or orthologue of said gene.

In one aspect, a Nic1 locus comprises at least SEQ ID No. 8 or a sequence which has at least 80% identity thereto (at least 85%, at least 90%, at least 95%, at least 97%, at least 98%, at least 99% identity thereto), or a functional variant or functional fragment or orthologue of said gene.

In another aspect, a Nic1 locus comprises a sequence or a chromosomal segment within 50, 100, 200, 300, 400, 500, 6000, 700, 800, 900, 1000, 1500, 2000, 3000, 4000, 5000, 6000, 7000, 8000, 9000, 10000, 15000, 20000, 30000 40000, 50000, 60000, 70000 nucleotides of a sequence selected from the group consisting of SEQ ID No. 5, SEQ ID No. 1, SEQ ID No. 9, SEQ ID No. 13, SEQ ID No. 17, SEQ ID No. 21, SEQ ID No. 25, SEQ ID No. 29, and SEQ ID No. 33.

As used herein, a “Nic1 gene” refers to a gene at or near a Nic1 locus and includes for example, the genes listed in Table 1; or a sequence which has at least 80% identity thereto (at least 85%, at least 90%, at least 95%, at least 97%, at least 98%, at least 99% identity thereto), or a functional variant or functional fragment or orthologue of said gene.

The at least one Nic1 ERF gene may be selected from the group comprising: a gene which encodes a polypeptide which comprises an amino acid sequence as set out in: SEQ ID No. 8; or SEQ ID No. 12; or SEQ ID No. 16; or SEQ ID No. 20; or SEQ ID No. 24; or SEQ ID No. 28; or SEQ ID No. 32 or a functional variant or functional fragment or orthologue thereof; or wherein the ERF gene comprises a nucleotide sequence as set out in: SEQ ID No. 5; or SEQ ID No. 9; or SEQ ID No. 13; or SEQ ID No. 17; or SEQ ID No. 21; or SEQ ID No. 25; or SEQ ID No. 29; or a functional variant or functional fragment or orthologue thereof.

Suitably, the at least one Nic1 ERF gene may be one, or two, or three, or four, or five, or six or seven genes selected from the group comprising: a gene which encodes a polypeptide which comprises an amino acid sequence as set out in: SEQ ID No. 8; or SEQ ID No. 12; or SEQ ID No. 16; or SEQ ID No. 20; or SEQ ID No. 24; or SEQ ID No. 28; or SEQ ID No. 32 or a functional variant or functional fragment or orthologue thereof; or wherein the ERF gene comprises a nucleotide sequence as set out in: SEQ ID No. 5; or SEQ ID No. 9; or SEQ ID No. 13; or SEQ ID No. 17; or SEQ ID No. 21; or SEQ ID No. 25; or SEQ ID No. 29; or a functional variant or functional fragment or orthologue thereof.

In one aspect, the at least one Nic1 ERF gene encodes a polypeptide which comprises an amino acid sequence as set out in: SEQ ID No. 8 or a functional variant or functional fragment or orthologue thereof; or wherein the Nic1 ERF gene comprises a nucleotide sequence as set out in SEQ ID No. 5 or a functional variant or functional fragment or orthologue thereof.

In one aspect, the activity or expression of at least one additional Nic1 ERF is modulated. Suitably, at least two, at least three, at least four, at least five, at least six, at least seven or at least eight additional Nic1 ERFs selected from Table 1 may also be modulated.

In one aspect, the at least one Nic1 ERF gene encodes a polypeptide which comprises an amino acid sequence as set out in: SEQ ID No. 8 or a functional variant or functional fragment or orthologue thereof; or the at least one Nic1 ERF gene comprises a nucleotide sequence as set out in SEQ ID No. 5 or a functional variant or functional fragment or orthologue thereof is modulated; and the activity or expression of at least one additional Nic1 ERF is modulated.

Suitably, the at least one additional Nic1 ERF may be selected from: a Nic1 ERF gene which encodes a polypeptide which comprises an amino acid sequence as set out in: SEQ ID No. 4; or SEQ ID No. 12; or SEQ ID No. 16; or SEQ ID No. 20; or SEQ ID No. 24; or SEQ ID No. 28; or SEQ ID No. 32; or SEQ ID No. 36 or a functional variant or functional fragment or orthologue thereof; or wherein the ERF gene comprises a nucleotide sequence as set out in: SEQ ID No. 1; SEQ ID No. 3, or SEQ ID No. 9; or SEQ ID No. 13; or SEQ ID No. 17; or SEQ ID No. 21; or SEQ ID No. 25; or SEQ ID No. 29; or SEQ ID No. 33 or a functional variant or functional fragment or orthologue thereof. Suitably, at least two, at least three, at least four, at least five, at least six, at least seven or at least eight additional Nic1 ERFs may be modulated.

As used herein, “Nic2” locus refers to any chromosomal position or location within or closely linked to the Nic2 region.

“Nic2 region” refers to a chromosomal segment as disclosed in WO2018/237107 (incorporated herein by reference), for example a chromosomal segment delimited by the markers SNP15 and SNP18/19 and having allele(s) associated with a low alkaloid (low-nicotine) trait. A forward primer for SNP15 is SEQ ID No. 304; a reverse primer for SNP15 is SEQ ID No. 305. A forward primer for SNP18 is SEQ ID No. 306; a reverse primer for SNP18 is SEQ ID No. 307. A forward primer for SNP19 is SEQ ID No. 308; a reverse primer for SNP19 is SEQ ID No. 309.

A “Nic2 mutation” refers to a mutation in a Nic2 locus.

In one aspect a Nic1 locus comprises one or more sequences selected from Table 1.

In one aspect a Nic2 locus comprises one or more sequences selected from the group consisting of: SEQ ID No. 69, SEQ ID No. 37, SEQ ID No. 41, SEQ ID No. 45, SEQ ID No. 49, SEQ ID No. 3, SEQ ID No. 57, SEQ ID No. 61, or SEQ ID No. 65, or a sequence which has at least 80% identity thereto (at least 85%, at least 90%, at least 95%, at least 97%, at least 98%, at least 99% identity thereto), or a functional variant or functional fragment or orthologue of said gene.

In one aspect, a Nic2 locus comprises at least SEQ ID No. 69 or a sequence which has at least 80% identity thereto (at least 85%, at least 90%, at least 95%, at least 97%, at least 98%, at least 99% identity thereto), or a functional variant or functional fragment or orthologue of said gene.

In another aspect, a Nic2 locus comprises a sequence or a chromosomal segment within 50, 100, 200, 300, 400, 500, 6000, 700, 800, 900, 1000, 1500, 2000, 3000, 4000, 5000, 6000, 7000, 8000, 9000, 10000, 15000, 20000, 30000 40000, 50000, 60000, 70000 nucleotides of a sequence selected from the group consisting of SEQ ID No. 69, SEQ ID No. 37, SEQ ID No. 41, SEQ ID No. 45, SEQ ID No. 49, SEQ ID No. 3, SEQ ID No. 57, SEQ ID No. 61, and SEQ ID No. 65.

As used herein, a “Nic2 gene” refers to a gene at or near a Nic2 locus and includes for example, the genes listed in Table 2; or a sequence which has at least 80% identity thereto (at least 85%, at least 90%, at least 95%, at least 97%, at least 98%, at least 99% identity thereto), or a functional variant or functional fragment or orthologue of said gene.

The at least one Nic2 ERF gene may be selected from the group comprising: a gene which encodes a polypeptide which comprises an amino acid sequence as set out in: SEQ ID No. 72; or SEQ ID No. 40; or SEQ ID No. 44; or SEQ ID No. 48; or SEQ ID No. 52; or SEQ ID No. 56; or SEQ ID No. 60, SEQ ID No. 64 or SEQ ID No. 68 or a functional variant or functional fragment or orthologue thereof; or wherein the ERF gene comprises a nucleotide sequence as set out in: SEQ ID No. 69; or SEQ ID No. 37; or SEQ ID No. 41; or SEQ ID No. 45; or SEQ ID No. 49; or SEQ ID No. 53; or SEQ ID No. 57; SEQ ID No. 61; SEQ ID No. 65; or a functional variant or functional fragment or orthologue thereof.

Suitably, the at least one Nic2 ERF gene may be one, or two, or three, or four, or five, or six or seven, or either or nine genes selected from Table 2.

In one aspect, the at least one Nic2 ERF gene encodes a polypeptide which comprises an amino acid sequence as set out in: SEQ ID No. 72 or a functional variant or functional fragment or orthologue thereof; or wherein the Nic1 ERF gene comprises a nucleotide sequence as set out in SEQ ID No. 59 or a functional variant or functional fragment or orthologue thereof.

The term “modulating” is used herein to mean either increasing or decreasing.

The term “increasing alkaloid content” is used herein to mean that the concentration and/or total alkaloid content in the product of the present invention (e.g. plant, part thereof (e.g. leaf), processed leaf or a product made from the plant (e.g. a delivery system)) is higher compared with a comparable product which has not been modified in accordance with the present invention.

The term “decreasing alkaloid content” is used herein to mean that the concentration and/or total alkaloid content in the product of the present invention (e.g. plant, part thereof (e.g. leaf), processed leaf or a product made from the plant (e.g. a delivery system)) is lower compared with a comparable product which has not be modified in accordance with the present invention.

In one aspect, the tobacco plants or parts thereof or tobacco cells according to the present invention comprise a total alkaloid level of less than 3%, less than 2.75%, less than 2.5%, less than 2.25%, less than 2%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1% or less than 0.05%.

In one aspect, the tobacco plants or parts thereof or tobacco cells according to the present invention comprise a nicotine level of less than 3%, less than 2.75%, less than 2.5%, less than 2.25%, less than 2%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1% or less than 0.05%. In one aspect, the tobacco plants or parts thereof or tobacco cells according to the present invention comprise an alkaloid level or nicotine level of less than 1%, less than 2%, less than 5%, less than 10%, less than 15%, less than 20%, less than 25%, less than 30%, less than 40%, less than 50%, less than 60%, less than 70%, less than 80% of the alkaloid level or nicotine level of a comparable plant or part thereof or cell.

In one embodiment the present invention provides a method of modulating (e.g. reducing) the content of tobacco-specific nitrosamine (TSNA) or a precursor of a TSNA in a plant (e.g. a tobacco plant) or a part thereof, the method comprising modifying said plant by modulating the activity or expression of at least one Nic3 gene. Suitably, the method may comprise modulating (e.g. decreasing) the activity or expression of at least one Nic3 gene, and optionally at least one Nic1 ERF gene and/or at least one Nic2 ERF gene.

In one embodiment the TSNA is N′nitrosonornicotine (NNN) and/or the precursor is nornicotine.

In one embodiment the TSNA may be one or more of group selected from: N′-nitrosonornicotine (NNN), N′nitrosoanatabine (NAT), N′-nitrosoanabasine (NAB) and 4-(methyl nitrosamino)-1-(3-pyridyl)-1-butanone (NNK).

In a preferred embodiment the TSNA is N′-nitrosonornicotine (NNN).

The TSNA may be measured in a processed tobacco, e.g. cured tobacco or reconstituted tobacco. In one embodiment the TSNA content is measured and/or modified (e.g. reduced) in a cured tobacco plant or part thereof (e.g. in cured tobacco leaf).

The term “tobacco-specific nitrosamine” or “TSNA” as used herein has its usual meaning in the art, namely a nitrosamine which is found only in delivery systems or other nicotine-containing products. Suitably the at least one tobacco-specific nitrosamine may be N′-nitrosonornicotine (NNN), 4-(methylnitrosamino)-1-(3-pyridyl)-1-butanone (NNK), N′-nitrosoanatabine (NAT) or N-nitrosoanabasine (NAB).

The term “precursor thereto” when used in relation to at least one tobacco-specific nitrosamine refers to one or more chemicals or compounds of a tobacco plant that give rise to the formation of a tobacco-specific nitrosamine or are involved in the nitrosation reaction leading to tobacco-specific nitrosamine production.

In one embodiment the precursor of the TSNA is one or more of the group selected from nornicotine, anabasine, anatabine, and an oxidised derivative of nicotine such as pseudooxynicotine (PON).

In a preferred embodiment the precursor of the TSNA is nornicotine.

The precursor of the TSNA (e.g. NNN, NNK, NAB and/or NAT) may be measured in green tobacco leaf, e.g. prior to processing, e.g. prior to curing. In one embodiment, the precursor of the TSNA (e.g. NNN, NNK, NAB and/or NAT) is measured and/or reduced in a green tobacco leaf, e.g. prior to processing, e.g. prior to curing.

In one embodiment carrying out a method and or use of the invention results in a reduction of at least one TSNA or a precursor thereto in the modified tobacco plant (or part thereof) or tobacco cell when compared to a tobacco plant (or part thereof) which has not been modified in accordance with the present invention.

The terms “reducing at least one TSNA or precursor thereto” or “reduction of at least one TSNA or precursor thereto” are used herein to mean that the concentration and/or total content of the at least one TSNA or precursor thereto in the product, method or use of the invention is lower in relation to a comparable product, method or use. For example, a comparable delivery system would be derived from a tobacco plant which had not been modified according to the present invention, but in which all other relevant features were the same (e.g. plant species, growing conditions, method of processing tobacco, etc.).

Any method known in the art for determining the concentration and/or levels of at least one TSNA or precursor thereto may be used. In particular such a method may comprise the addition of deuterium labelled internal standard, an aqueous extraction and filtration, followed by analysis using reversed phase high performance liquid chromatography with tandem mass spectrometry (LC-MS/MS) may be used. Other examples for determining the concentration and/or level of a precursor to a tobacco-specific nitrosamine include a method such as the one detailed in CORESTA recommended method CRM-72: Determination of Tobacco Specific Nitrosamines in Tobacco and Delivery systems by LC-MS/MS; CRM being developed into ISO/DIS 21766 or Wagner et al. Analytical Chemistry (2005), 77(4), 1001-1006 all of which are incorporated herein by reference.

Suitably the concentration and/or total content of the at least one tobacco-specific nitrosamine or precursor thereto may be reduced by carrying out a method and/or use of the present invention.

Suitably the concentration and/or level of the at least one tobacco-specific nitrosamine or precursor thereto may be reduced in a tobacco plant of the invention (e.g. obtainable or obtained by a method and/or use of the invention) when compared to the concentration and/or level of the at least one tobacco-specific nitrosamine(s) or precursor thereto in a tobacco plant which has not been modified in accordance with present invention.

The concentration and/or total content of the at least one tobacco-specific nitrosamine(s) or precursor thereto may be reduced in a tobacco leaf, harvested leaf, processed tobacco leaf, delivery system or combinations thereof obtainable or obtained from a tobacco plant (or part of a tobacco plant or a tobacco cell culture) of the invention when compared with a tobacco leaf, harvested leaf, processed tobacco leaf, delivery system or combinations thereof obtainable or obtained from a tobacco plant (or part of a tobacco plant or a tobacco cell culture) which has not been modified in accordance with the present invention.

Suitably the concentration and/or total content of the at least one tobacco-specific nitrosamine or precursor thereto may be reduced in a processed tobacco leaf.

Suitably the concentration and/or level of the at least one tobacco-specific nitrosamine or precursor thereto may be reduced in a delivery system.

In one embodiment the at least one tobacco-specific nitrosamine or precursor thereto may be reduced by at least about 1%, at least about 3%, at least about 5%, at least about 10%, at least about 20%, at least about 30%, at least about 40%, at least about 50%, at least about 60%, at least about 70%, at least about 80% or at least about 90%. In some embodiments the at least one tobacco-specific nitrosamine or precursor thereto may be reduced by between about 5% and about 95%, by between about 10% and about 90%, by between 20% and about 80%, by between 30% and about 70%, or by between about 40% and 60%.

In relation to processed (e.g. cured) tobacco leaf (e.g. cured or reconstituted), the at least one tobacco-specific nitrosamine or precursor thereto may be reduced by between about 5000 ng/g and about 50 ng/g, by between about 4000 ng/g and about 100 ng/g, by between about 3000 ng/g and 500 ng/g or by between 2000 ng/g and 1000 ng/g. In some embodiments the at least one tobacco-specific nitrosamine or precursor thereto may be reduced by at least about 5000 ng/g, at least about 4000 ng/g, at least about 3000 ng/g, at least about 2000 ng/g, at least about 1000 ng/g, at least about 500 ng/g, at least about 100 ng/g or at least about 50 ng/g.

The term “a comparable product” as defined herein would be one derived from a plant (e.g. a tobacco plant) which had not been modified according to the present invention, but in which all other relevant features were the same (e.g. plant species, growing conditions, method of processing the plant, e.g. tobacco, etc.). The comparable product according to the present invention may mean a tobacco plant cell or a plant (e.g. a tobacco plant) or a part thereof, such as a leaf (e.g. a tobacco leaf), a harvested leaf (e.g. a harvested tobacco leaf), a cut harvested leaf (e.g. a cut harvested tobacco leaf), a processed leaf (e.g. a processed tobacco leaf) or plant propagation material (e.g. tobacco plant propagation material), or a product comprising said plant or part therefore, e.g. a delivery system or combinations thereof obtainable or obtained from a plant which has not been modified in accordance with the present invention, e.g. to modulate the activity or expression of a Nic3 gene (or a Nic3 gene in combination with one or more Nic1 ERF genes in combination with one or more Nic2 ERF genes). Comparable products may also be known as controls or as wild-type.

The term “unmodified plant” as defined herein would be a plant (e.g. a tobacco plant) which had not been modified according to the present invention, to modulate the activity or expression of a Nic3 gene and in which all other relevant features were the same (e.g. plant species, growing conditions, method of processing tobacco, etc.).

The “activity or expression” of a Nic3 gene (or a Nic1 ERF gene or Nic2 ERF gene) may refer to the level of transcription, translation i.e. protein expression, or the activity of the protein encoded by the Nic3 gene (or the Nic1 ERF or Nic2 ERF gene respectively). The activity of a Nic3 gene relates to its ability to function as a regulator of alkaloid biosynthesis and in particular nicotine biosynthesis. The activity of a Nic1 ERF gene (or a Nic2 ERF gene) relates to its ability to function as a transcription factor in the biosynthesis of alkaloids. The activity of a Nic3 gene (or a Nic1 ERF gene or a Nic2 ERF gene) may be determined by measuring the products of alkaloid synthesis i.e. by measuring alkaloid content.

According to one aspect of the invention, gene expression may be decreased (or inhibited) by inhibiting transcription and/or translation. In one embodiment the activity or expression of a gene may refer to the level of transcription, i.e. the amount of mRNA produced, or translation i.e. the level or amount of protein produced.

In some embodiments, the modulation of alkaloid content refers to an increase in alkaloid content wherein the activity or expression of at least one Nic3 gene is modulated.

In some embodiments, the modulation of alkaloid content refers to a decrease in alkaloid content wherein the activity or expression of at least one Nic3 gene is modulated.

In some embodiments, the modulation of alkaloid content refers to an increase in alkaloid content wherein the activity or expression of at least one Nic3 gene, and optionally the activity or expression of at least one Nic1 ERF gene and/or Nic2 ERF is modulated in combination.

In some embodiments, the modulation of alkaloid content refers to a decrease in alkaloid content wherein the activity or expression of at least one Nic3 gene, and optionally the activity or expression of at least one Nic1 ERF gene and/or Nic2 ERF is modulated in combination.

In a further aspect, the alkaloid content is measured from leaves. In one aspect the alkaloid content is measured from green leaves. In a further aspect, the alkaloid content is measured from cured leaves, e.g. air-cured, flue-cured, fire-cured or sun-cured leaves. In a further aspect, the alkaloid content is measured from flue-cured leaves. In a further aspect, the alkaloid content is measured from air-cured leaves.

The term “alkaloid content” is used herein to mean the concentration and/or total amount of the entire group of compounds classified as alkaloids. Alkaloids typically present in tobacco include nicotine, anatabine, anabasine, myosmine and nornicotine. In one embodiment the content of one or more alkaloids selected from nicotine, anatabine, anabasine, myosmine and nornicotine is modulated. In one embodiment the content of one or more alkaloids selected from nicotine, anatabine, anabasine, myosmine and nornicotine is reduced. In one embodiment the content of one or more alkaloids selected from nicotine, anatabine, anabasine and nornicotine is increased.

Suitably nicotine content is modulated. In one embodiment the nicotine content is reduced.

Any method known in the art for determining the concentration and/or total content of alkaloids may be used. One preferred method for analysing alkaloid content involves the analysis by gas chromatography-flame ionization detection method (GC-FID).

In one embodiment there is provided a method for producing a plant (e.g. a tobacco plant) or part thereof, a plant propagation material (e.g. a tobacco plant propagation material), a cell (e.g. a tobacco cell), a leaf (e.g. a tobacco leaf), a harvested leaf (e.g. a harvested tobacco leaf), a cut harvested leaf (e.g. a cut harvested tobacco leaf), a processed leaf (e.g. a processed tobacco leaf), a cut and processed leaf (e.g. a cut and processed tobacco leaf), a product comprising said plant or part thereof (e.g. a delivery system) or combinations thereof obtainable or obtained by a plant of the invention which has modulated alkaloid content, the method comprising modifying said tobacco to modulate the activity or expression of a Nic3 gene or the combination of a Nic3 gene and at least one Nic1 ERF gene and/or at least one Nic2 ERF gene. The modulated alkaloid content may be determined by comparing the alkaloid content in the plant (e.g. tobacco plant) or part thereof, plant propagation material (e.g. tobacco plant propagation material), a cell (e.g. a tobacco cell), leaf (e.g. tobacco leaf), harvested leaf (e.g. a harvested tobacco leaf), cut harvested leaf (e.g. a cut harvested tobacco leaf), processed leaf (e.g. processed tobacco leaf), cut and processed leaf (e.g. cut and processed tobacco leaf), a product comprising a plant or part thereof of the present invention, e.g. a delivery system, or combinations thereof with a comparable product.

Suitably the alkaloid content may be modulated in a plant, e.g. a tobacco plant e.g. modified tobacco plant. Suitably the alkaloid content may be modulated in a leaf (e.g. a tobacco leaf e.g. a tobacco leaf from a modified tobacco plant). Suitably the alkaloid content may be modulated in a harvested leaf (e.g. a harvested tobacco leaf from a modified tobacco plant). Suitably the alkaloid content may be modulated in a cut harvested leaf (e.g. a cut harvested tobacco leaf from a modified tobacco plant). Suitably the alkaloid content may be modulated in a processed leaf (e.g. a processed tobacco leaf e.g. a processed tobacco leaf from a modified tobacco plant). Suitably the alkaloid content may be modulated in a cut and processed leaf (e.g. a cut and processed tobacco leaf e.g. a cut and processed tobacco leaf from a modified tobacco plant). Suitably the alkaloid content may be modulated in a cured leaf (e.g. cured a tobacco leaf from a modified tobacco plant). Suitably the alkaloid content may be modulated in an extract of a green leaf (e.g. a green tobacco leaf from a modified tobacco plant). Suitably the alkaloid content may be modulated in a product comprising the plant of the present invention or part thereof (e.g. a delivery system, for example a delivery system produced from a modified tobacco plant or part thereof).

Suitably the alkaloid content may be modulated in any one of the above products or combinations thereof. Suitably the modulation of alkaloid content described above may be an increase in alkaloid content. Suitably the modulation of alkaloid content described above may be a decrease in alkaloid content.

In one embodiment the content of one or more alkaloids selected from nicotine, anatabine, anabasine, myosmine and nornicotine is decreased.

Suitably the modulation of alkaloid content described above may be a decrease in nicotine content.

In one embodiment the nicotine content of a modified tobacco cell, or modified plant or part thereof (e.g. tobacco plant), plant propagation material (e.g. tobacco plant propagation material), leaf (e.g. tobacco leaf), harvested leaf (e.g. harvested tobacco leaf), cut harvested leaf (e.g. cut harvested tobacco leaf), processed leaf (e.g. processed tobacco leaf), cut and processed leaf (e.g. cut and processed tobacco leaf) or delivery system from a modified tobacco plant is decreased.

In one embodiment the alkaloid content of a plant (e.g. tobacco plant) or part thereof may be modulated by at least 2, 3, 4, 5, 6, 7, 8, 9 or 10, fold when compared to the alkaloid content of a plant (e.g. tobacco plant) or part thereof, respectively, which has not been modified to modulate the activity or expression of at least one Nic3 gene (or at least one Nic3 gene in combination with at least one Nic1 ERF and/or at least one Nic2 ERF gene) which has been grown under similar growth conditions. Suitably the alkaloid content may be modulated (e.g. reduced) by about 2 fold to about 10 fold, preferably about 3 fold to about 10 fold, suitably about 3 fold to about 5 fold. Suitably the modification may be an increase or a decrease in alkaloid content. Suitably the modulation (e.g. reduction) may be of one or more alkaloids selected from nicotine, anatabine, anabasine, myosmine and nornicotine. Suitably, nicotine content is reduced.

In one embodiment of the invention the alkaloid content of a tobacco plant cell or plant (e.g. a tobacco plant) or part thereof may be modulated by 1%, 2%, 5%, 8%, 10%, 12%, 15%, 20%, 25%, 30%, 40%, 50%, 60%, 70%, 80% or 90% in comparison to a cell or plant (e.g. a tobacco plant) or part thereof which has not been modified according to the present invention. The modulation may be an increase or a decrease in alkaloid content when compared to an unmodified plant (e.g. a tobacco plant) or part thereof. Suitably the modulation may be of total alkaloid content. Suitably the modulation may be of one or more alkaloids selected from nicotine, anatabine, anabasine, myosmine and nornicotine. Suitably nicotine content is reduced.

In one embodiment the method or use results in modulated alkaloid content in comparison to a plant (e.g. a tobacco plant) or part thereof or cell which has not been modified to modulate the activity or expression of a Nic3 gene (or a combination of a Nic3 gene and a Nic1 ERF gene and/or a Nic2 ERF gene) and more particularly as compared to, or relative to, the expression by a plant (e.g. a tobacco plant) in the absence of the introduced modification.

In one embodiment the method or use results in modulated alkaloid content in comparison to a plant (e.g. a tobacco plant) or part thereof or cell which has not been modified to introduce a mutation to a Nic3 gene (or a combination of a Nic3 gene and a Nic1 ERF gene and/or a Nic2 ERF gene) and more particularly as compared to, or relative to, a plant (e.g. a tobacco plant) or part thereof or cell in the absence of the introduced modification.

In an embodiment a plant (e.g. a tobacco plant) or part thereof or cell has been modified to achieve a modulation in alkaloid content in comparison to a plant (e.g. a tobacco plant) or part thereof, respectively, which has not been modified to modulate the activity or expression of the at least one Nic3 gene (or at least one Nic3 gene and at least one Nic1 ERF gene and/or at least one Nic2 ERF gene). References herein to Nic3 and Nic1 and/or Nic2 (when referring to loci or genes) concern the options i) Nic3 and Nic1, ii) Nic3 and Nic 2, and iii) Nic3, Nic1 and Nic 2.

The term “modifying” or “modified” as used herein means a cell (e.g. tobacco cell), plant (e.g. a tobacco plant) that has been altered or changed. The present invention comprises the modification of plants using techniques for genetic modification of plants or non-genetic modification of plants. Such methods are well known in the art and examples of genetic modification techniques include transformation, transgenics, cisgenics, and gene editing methods. Examples of non-genetic modification techniques include fast-neutron mutagenesis, chemical mutagenesis e.g. ethyl methanesulfonate (EMS) mutagenesis and modern population analysis approaches.

In one embodiment a natural variant which has a modified Nic3 gene (or a modified Nic3 gene in combination with at least one modified Nic1 ERF gene and/or at least one modified Nic2 ERF gene) is selected and that trait or gene is bred into a second plant which has commercially desirable traits.

In one embodiment the cell or plant (e.g. a tobacco plant) according to the invention may be a transgenic cell plant.

In another embodiment the cell plant (e.g. a tobacco plant) according to the invention may be a non-transgenic cell or plant.

Suitably the mutation in the at least one Nic3 gene according to the present invention may not be present in K326.

Suitably the mutation in the at least one Nic3 gene according to the present invention may not be present in Green Briar.

Suitably the modulation of the at least one Nic3 gene is not present in Burley 21.

In some embodiments a modification which modulates the activity or expression of at least one Nic3 gene and thereby modulates alkaloid content is selected from the group consisting of: decreasing, preventing or attenuating transcription, translation or expression of the at least one Nic3 gene (or the combination of at least one Nic3 gene and at least one Nic1 ERF gene and/or at least one Nic2 ERF gene);

inhibiting synthesis of the polypeptide encoded by at least one Nic3 gene (or the combination of at least one Nic3 gene and at least one Nic1 ERF gene and/or at least one Nic2 ERF gene in combination), or its release from intracellular stores; or

increasing the rate of degradation of the polypeptide encoded by at least one Nic3 gene (or the combination of at least one Nic3 gene and at least one Nic1 ERF gene and/or at least one Nic2 ERF gene in combination).

In one embodiment the modification which decreases the activity or expression of at least one Nic3 gene (or one or more modifications which decreases the activity of at least one Nic3 gene and at least one Nic1 ERF gene and/or at least one Nic2 ERF gene in combination) comprises a mutation in one or more genes.

In one embodiment the mutation deletes the entire one or more Nic3 gene(s). Suitably, a mutation may delete one or more Nic1 ERF genes. Suitably, a mutation may delete one or more Nic2 ERF genes.

In one embodiment the one or more Nic3 gene(s) may comprise one or more mutations within the gene(s). Suitably the one or more mutations result in reduced or eliminated gene activity in the mutated gene. In one embodiment the one or more mutations results in an inactive gene. In one embodiment the mutation results in an amino acid substitution. In one embodiment the mutation is a nonsense mutation. Suitably, the mutation may inhibit the normal function of the protein encoded by the gene, such as a Nic3 gene, for example inhibiting DNA binding in the case of a transcription factor.

By way of example, the present method may comprise:
providing a mutation in a nucleic acid sequence which encodes a protein listed in Table 3, or an amino acid sequence which has at least 70% (preferably at least 80%, preferably at least 90%, preferably at least 96%, preferably at least 98%) sequence identity thereto sequence identity thereto;
providing a mutation in a promoter of a nucleic acid sequence which encodes a protein comprising the amino acid sequence shown in Table 3, or an amino acid sequence which has at least 70% (preferably at least 80%, preferably at least 90%, preferably at least 96%, preferably at least 98%) sequence identity thereto
providing a mutation in a nucleic acid sequence of a Nic3 gene listed in Table 3, or a nucleotide sequence which has at least 70% (preferably at least 80%, preferably at least 90%, preferably at least 96%, preferably at least 98%) sequence identity thereto;
providing a mutation in a promoter of a nucleic acid sequence of a Nic3 gene listed in Table 3 or a nucleotide sequence which has at least 70% (preferably at least 80%, preferably at least 90%, preferably at least 96%, preferably at least 98%) sequence identity thereto;
providing an antisense RNA, siRNA or miRNA which reduces the level of nucleic acid sequence encoding a Nic3 protein listed in Table 3 or an amino acid sequence which has at least 70% (preferably at least 80%, preferably at least 90%, preferably at least 96%, preferably at least 98%) sequence identity thereto;
providing an antisense RNA, siRNA or miRNA which reduces the level of a nucleic acid sequence listed in Table 3, or a sequence which has at least 70% (preferably at least 80%, preferably at least 90%, preferably at least 96%, preferably at least 98%) sequence identity thereto. Suitably the protein listed in Table 3 or amino acid sequence shown in Table 3 is selected from SEQ ID Nos. 75, 120, 127 and 129 (or their related sequences as described hereinbefore). Suitably the gene or sequence listed in Table 3 is selected from SEQ ID Nos. 73, 118, 124 and 127 (or their related sequences as described hereinbefore).
In one embodiment the at least one mutation (or one or more mutation) in the Nic3 locus is in a Nic3 gene selected from SEQ ID No. 73, 118, 124 or 127 or a sequence which has at least 90% identity thereto or a functional variant or functional fragment or orthologue of said gene and said at least one mutation in the Nic3 gene is selected from:
   i) a mutation in the Nic3 gene SEQ ID No. 73 or a sequence which has at least 90% identity thereto or a functional variant or functional fragment or orthologue of said gene that results in a mutation in amino acid residues 74 to 258 or 483-538 of SEQ ID No. 75 or a sequence which has at least 90% identity thereto, or a functional variant or functional fragment or orthologue of said polypeptide;
   ii) a mutation in the Nic3 gene SEQ ID No. 118 or a sequence which has at least 90% identity thereto or a functional variant or functional fragment or orthologue of said gene that results in a mutation in amino acid residues 120-584 of SEQ ID No. 120 or a sequence which has at least 90% identity thereto, or a functional variant or functional fragment or orthologue of said polypeptide;
   iii) a mutation in the Nic3 gene SEQ ID No. 124 or a sequence which has at least 90% identity thereto or a functional variant or functional fragment or orthologue of said gene that results in a mutation in amino acid residues 166-406 or 483-970 of SEQ ID No. 126 or a sequence which has at least 90% identity thereto, or a functional variant or functional fragment or orthologue of said polypeptide; and
   iv) a mutation in the Nic3 gene SEQ ID No. 127 or a sequence which has at least 90% identity thereto or a functional variant or functional fragment or orthologue of said gene that results in a mutation in amino acid residues 171-406 or 509-967 of SEQ ID No. 129 or a sequence which has at least 90% identity thereto, or a functional variant or functional fragment or orthologue of said polypeptide.

Amino acid residues 74 to 258 of SEQ ID No. 75 provide the N-terminal MYC domain of the MYC transcription factor. Amino acid residues 483-538 of SEQ ID No. 75 provide a bHLH domain with several DNA-binding sites (which suitably provide sites for mutation), namely at amino acid residues 488, 489, 492, 493, 500, 517 and 518. Amino acid residues 120 to 584 of SEQ ID No. 120 provide an LRR domain. Amino acid residues 166 to 406 of SEQ ID No. 124 provide an NB-ARC domain and amino acid residues 483-970 provide an LRR domain. Amino acid residues 171-406 of SEQ ID No. 129 provide an NB-ARC domain and amino acid residues 509-967 provide an LRR domain. The site of the mutation may also be described by reference to the encoding nucleotide, e.g. amino acid residues 74 to 258 of SEQ ID No. 75 are encoded by nucleotides 631-1185 of SEQ ID No. 73.

As described above the mutation is made in a specific gene or a related sequence (as defined herein) and provides a mutation in the recited amino acids or in a related sequence (as defined herein). The related sequences correspond to one another. For example, if a mutation is made in a sequence which has 90% sequence identity to SEQ ID No. 75, the resulting mutant is made in the context of that related sequence, i.e. provides a sequence with the same sequence identity before taking into account the one or more mutations that have been introduced. Where a related sequence is mutated a mutation is made in the sequence corresponding to the above recited domains, i.e. taking into account any changes in amino acid number resulting from the generation of a related sequence.

In one embodiment the mutation may be a deletion. By way of example the domains described above may be deleted in part or in their entirety. In one embodiment the mutation may be an insertion. In one embodiment the mutation may introduce an early stop codon. In one embodiment the target site is unique to the target Nic3 gene and does not exist in other genes.

In one embodiment the mutants have reduced total alkaloid and/or reduced nicotine levels.

In one embodiment, the present invention provides one or more mutations in a Nic1 ERF gene encoding a polypeptide comprising (or consisting of) amino acid sequence as shown in Table 1, or a sequence with at least 90%, preferably at least 96%, identity therewith.

Suitably, the present invention may provide one or more mutations in a Nic1 ERF gene encoding a polypeptide comprising (or consisting of) amino acid sequence SEQ ID No. 8 or a sequence with at least 90%, preferably at least 96%, identity therewith.

Suitably, the present invention may provide one or more mutations in a Nic1 ERF gene comprising (or consisting of) the nucleotide sequence as set out in: SEQ ID No. 5 or a sequence with at least 90%, preferably at least 96%, identity therewith.

By way of example, the present method may comprise:
providing a mutation in a nucleic acid sequence which encodes a protein comprising the amino acid sequence shown SEQ ID No. 8; or SEQ ID No. 4; or SEQ ID No. 12; or SEQ ID No. 16; or SEQ ID No. 20; or SEQ ID No. 24; or SEQ ID No. 28; or SEQ ID No. 32; or SEQ ID No. 36 or an amino acid sequence which has at least 70% (preferably at least 80%, preferably at least 90%, preferably at least 96%, preferably at least 98%) sequence identity thereto;
providing a mutation in a promoter of a nucleic acid sequence which encodes a protein comprising the amino acid sequence shown as SEQ ID No. 8; or SEQ ID No. 4; or SEQ ID No. 12; or SEQ ID No. 16; or SEQ ID No. 20; or SEQ ID No. 24; or SEQ ID No. 28; or SEQ ID No. 32; or SEQ ID No. 36 or an amino acid sequence which has at least 70% (preferably at least 80%, preferably at least 90%, preferably at least 96%, preferably at least 98%) sequence identity thereto
providing a mutation in a nucleic acid sequence of an ERF gene which comprises SEQ ID No. 5; or SEQ ID No. 1; or SEQ ID No. 3; or SEQ ID No. 9; or SEQ ID No. 13; or SEQ ID No. 17; or SEQ ID No. 21; or SEQ ID No. 25; or SEQ ID No. 29; or SEQ ID No. 33 or a nucleotide sequence which has at least 70% (preferably at least 80%, preferably at least 90%, preferably at least 96%, preferably at least 98%) sequence identity thereto;
providing a mutation in a promoter of a nucleic acid sequence of an ERF gene which comprises SEQ ID No. 5; or SEQ ID No. 1; or SEQ ID No. 3; or SEQ ID No. 9; or SEQ ID No. 13; or SEQ ID No. 17; or SEQ ID No. 21; or SEQ ID No. 25; or SEQ ID No. 29; or SEQ ID No. 33 or a nucleotide sequence which has at least 70% (preferably at least 80%, preferably at least 90%, preferably at least 96%, preferably at least 98%) sequence identity thereto;
providing an antisense RNA, siRNA or miRNA which reduces the level of nucleic acid sequence encoding a protein comprising the amino acid sequence shown as SEQ ID No. 8; or SEQ ID No. 4; or; or SEQ ID No. 12; or SEQ ID No. 16; or SEQ ID No. 20; or SEQ ID No. 24; or SEQ ID No. 28; or SEQ ID No. 32; or SEQ ID No. 36 or an amino acid sequence which has at least 70% (preferably at least 80%, preferably at least 90%, preferably at least 96%, preferably at least 98%) sequence identity thereto;
providing an antisense RNA, siRNA or miRNA which reduces the level of nucleic acid sequence SEQ ID No. 5; or SEQ ID No. 1; or SEQ ID No. 3, or; or SEQ ID No. 9; or SEQ ID No. 13; or SEQ ID No. 17; or SEQ ID No. 21; or SEQ ID No. 25; or SEQ ID No. 29; or SEQ ID No. 33 or a nucleotide sequence which has at least 70% (preferably at least 80%, preferably at least 90%, preferably at least 96%, preferably at least 98%) sequence identity thereto.

In one embodiment one or more Nic1 ERF gene(s) and/or one or more Nic2 ERF gene(s) are modulated (e.g. mutated).

Suitably any one of the Nic3 gene and/or Nic1 ERF gene modifications (e.g. mutations) taught herein may be used in combination with one or more modifications of a Nic2 ERF gene wherein the Nic2 ERF gene encodes a polypeptide which comprises an amino acid sequence as set out in: SEQ ID No. 72; or SEQ ID No. 40; or SEQ ID No. 44; or SEQ ID No. 48; or SEQ ID No. 52; or SEQ ID No. 56; or SEQ ID No. 60; or SEQ ID No. 64; or SEQ ID No. 68; or a functional variant or functional fragment or orthologue thereof; or the Nic2 ERF gene comprises a nucleotide sequence as set out in: SEQ ID No. 69; SEQ ID No. 37; or SEQ ID No. 41; or SEQ ID No. 45; or SEQ ID No. 49; or SEQ ID No. 53; or SEQ ID No. 57; or SEQ ID No. 61; or SEQ ID No. 65; or a functional variant or functional fragment or orthologue thereof.

By way of example, the present method may comprise:
providing a mutation in a nucleic acid sequence which encodes a protein comprising the amino acid sequence shown as SEQ ID No. 72; or SEQ ID No. 40; or SEQ ID No. 44; or SEQ ID No. 48; or SEQ ID No. 52; or SEQ ID No. 56; or SEQ ID No. 60; or SEQ ID No. 64; or SEQ ID No. 68; or an amino acid sequence which has at least 70% (preferably at least 80%, preferably at least 90%, preferably at least 96%, preferably at least 98%) sequence identity thereto;
providing a mutation in a promoter of a nucleic acid sequence which encodes a protein comprising the amino acid sequence shown as SEQ ID No. 72; or SEQ ID No. 40; or SEQ ID No. 44; or SEQ ID No. 48; or SEQ ID No. 52; or SEQ ID No. 56; or SEQ ID No. 60; or SEQ ID No. 64; or SEQ ID No. 68; or an amino acid sequence which has at least 70% (preferably at least 80%, preferably at least 90%, preferably at least 96%, preferably at least 98%) sequence identity thereto;
providing a mutation in a nucleic acid sequence of an ERF gene which comprises SEQ ID No. 69; or SEQ ID No. 37; or SEQ ID No. 41; or SEQ ID No. 45; or SEQ ID No. 49; or SEQ ID No. 53; or SEQ ID No. 57; or SEQ ID No. 61; or SEQ ID No. 65; or a nucleotide sequence which has at least 70% (preferably at least 80%, preferably at least 90%, preferably at least 96%, preferably at least 98%) sequence identity thereto;
providing a mutation in a promoter of a nucleic acid sequence of an ERF gene which comprises SEQ ID No. 69; or SEQ ID No. 37; or SEQ ID No. 41; or SEQ ID No. 45; or SEQ ID No. 49; or SEQ ID No. 53; or SEQ ID No. 57; or SEQ ID No. 61; or SEQ ID No. 65; or a nucleotide sequence which has at least 70% (preferably at least 80%, preferably at least 90%, preferably at least 96%, preferably at least 98%) sequence identity thereto;
providing an antisense RNA, siRNA or miRNA which reduces the level of nucleic acid sequence encoding a protein comprising the amino acid sequence shown as SEQ ID No. 72; or SEQ ID No. 40; or SEQ ID No. 44; or SEQ ID No. 48; or SEQ ID No. 52; or SEQ ID No. 56; or SEQ ID No. 60; or SEQ ID No. 64; or SEQ ID No. 68; or an amino acid sequence which has at least 70% (preferably at least 80%, preferably at least 90%, preferably at least 96%, preferably at least 98%) sequence identity thereto;
providing an antisense RNA, siRNA or miRNA which reduces the level of nucleic acid sequence SEQ ID No. 69; or SEQ ID No. 37; or SEQ ID No. 41; or SEQ ID No. 45; or SEQ ID No. 49; or SEQ ID No. 53; or SEQ ID No. 57; or SEQ ID No. 61; or SEQ ID No. 65; or a nucleotide sequence which has at least 70% (preferably at least 80%, preferably at least 90%, preferably at least 96%, preferably at least 98%) sequence identity thereto.

In one embodiment there is used in combination a mutation in at least one Nic1 ERF gene, selected from the group consisting of one or more mutations in a nucleic acid sequence which encodes a protein comprising the amino acid sequence shown as SEQ ID No. 4; or SEQ ID No. 8; or SEQ ID No. 12; or SEQ ID No. 16; or SEQ ID No. 20; or SEQ ID No. 24; or SEQ ID No. 28; or SEQ ID No. 32; or SEQ ID No. 36 or an amino acid sequence which has at least 70% (preferably at least 80%, preferably at least 90%, preferably at least 96%, preferably at least 98%) sequence identity thereto, or one or more mutations in a nucleic acid sequence of an ERF gene which comprises SEQ ID No. 1; or SEQ ID No. 3; or SEQ ID No. 5; or SEQ ID No. 9; or SEQ ID No. 13; or SEQ ID No. 17; or SEQ ID No. 21; or SEQ ID No. 25; or SEQ ID No. 29; or SEQ ID No. 33 or a nucleotide sequence which has at least 70% (preferably at least 80%, preferably at least 90%, preferably at least 96%, preferably at least 98%) sequence identity thereto); and/or a mutation in at least one Nic2 ERF gene, particularly one or mutations in the nucleotide sequence encoding the amino acid sequence SEQ ID No. 40, SEQ ID No. 44, SEQ ID No. 48, SEQ ID No. 52 or SEQ ID No. 56, SEQ ID No. 64, SEQ ID No. 68 or SEQ ID No. 72 or an amino acid sequence which has at least 70% (preferably at least 80%, preferably at least 90%, preferably at least 96%, preferably at least 98%) sequence identity thereto, or one or more mutations in a nucleic acid sequence which comprises SEQ ID No. 37, SEQ ID No. 41, SEQ ID No. 45, SEQ ID No. 49 or SEQ ID No. 53, or SEQ ID No. 57 or SEQ ID No. 61 or SEQ ID No. 65 or SEQ ID No. 69 or a nucleotide sequence which has at least 70% (preferably at least 80%, preferably at least 90%, preferably at least 96%, preferably at least 98%) sequence identity thereto, more particularly, the Nic2 ERF mutation is one or mutations in the nucleotide sequence encoding the amino acid sequence SEQ ID No. 72 or an amino acid sequence which has at least 70% (preferably at least 80%, preferably at least 90%, preferably at least 96%, preferably at least 98%) sequence identity thereto, or in nucleotide sequence SEQ ID No. 69 or a nucleotide sequence which has at least 70% (preferably at least 80%, preferably at least 90%, preferably at least 96%, preferably at least 98%) sequence identity thereto.

In one embodiment there is used, optionally in combination a mutation in at least one Nic1 ERF gene consisting of one or more mutations in a nucleic acid sequence which encodes a protein comprising the amino acid sequence shown as SEQ ID No. 8; or an amino acid sequence which has at least 70% (preferably at least 80%, preferably at least 90%, preferably at least 96%, preferably at least 98%) sequence identity thereto, or one or more mutations in a nucleic acid sequence of an ERF gene which comprises SEQ ID No. 5; or a nucleotide sequence which has at least 70% (preferably at least 80%, preferably at least 90%, preferably at least 96%, preferably at least 98%) sequence identity thereto); and/or a mutation in at least one Nic2 ERF gene, particularly one or mutations in the nucleotide sequence encoding the amino acid sequence SEQ ID No. 40, SEQ ID No. 44, SEQ ID No. 48, SEQ ID No. 52, SEQ ID No. 56, SEQ ID No. 60, SEQ ID No. 64, SEQ ID No. 68 or SEQ ID No. 72 or an amino acid sequence which has at least 70% (preferably at least 80%, preferably at least 90%, preferably at least 96%, preferably at least 98%) sequence identity thereto, or one or more mutations a nucleotide sequence which comprises SEQ ID No. 37, SEQ ID No. 41, SEQ ID No. 45, SEQ ID No. 49, SEQ ID No. 53, SEQ ID No. 57, SEQ ID No. 61, SEQ ID No. 65 or SEQ ID No. 69 or a nucleotide sequence which has at least 70% (preferably at least 80%, preferably at least 90%, preferably at least 96%, preferably at least 98%) sequence identity thereto.

In one embodiment there is used optionally in combination a mutation in at least one Nic1 ERF gene consisting of one or more mutations in a nucleic acid sequence which encodes a protein comprising the amino acid sequence shown as SEQ ID No. 8; or an amino acid sequence which has at least 70% (preferably at least 80%, preferably at least 90%, preferably at least 96%, preferably at least 98%) sequence identity thereto, or one or more mutations in a nucleic acid sequence of an ERF gene which comprises SEQ ID No. 5; or a nucleotide sequence which has at least 70% (preferably at least 80%, preferably at least 90%, preferably at least 96%, preferably at least 98%) sequence identity thereto); and/or a mutation in at least one Nic2 ERF gene consisting of one or more mutations in a nucleotide sequence which encodes the amino acid sequence shown as SEQ ID No. 72 or an amino acid sequence which has at least 70% (preferably at least 80%, preferably at least 90%, preferably at least 96%, preferably at least 98%) sequence identity thereto, or one or more mutations in a nucleotide sequence shown as SEQ ID No. 69 or a nucleotide sequence which has at least 70% (preferably at least 80%, preferably at least 90%, preferably at least 96%, preferably at least 98%) sequence identity thereto.

One or more Nic2 ERF genes may be one, or two, or three, or four, or five, or six, or seven or eight or nine Nic2 ERF genes selected from Table 2.

In some embodiments a modification which decreases the activity or expression of at least one Nic3 gene (or of at least one Nic3 gene and at least one Nic1 ERF gene and/or at least one Nic2 ERF gene in combination) and thereby decreases alkaloid content is one or more selected from the group consisting of a point mutation, a deletion, an insertion, a duplication, and an inversion in one or more genes. Suitably the modification is introduced by a method selected from random mutagenesis and targeted mutagenesis. Suitably the modification may be introduced by a targeted mutagenesis method selected from meganuclease, zinc finger nuclease, TALEN, gene editing and CRISPR for example.

As used herein, the term “mutation” encompasses a natural genetic variant or an engineered variant.

A mutation refers to an inheritable genetic modification introduced to a plant or part thereof or cell, which alters the activity or expression of a product encoded by a gene. These modifications may be in any sequence which controls the activity or expression of a gene, for example in a promoter, 5′ UTR, exon, intron, 3′UTR, or terminator region. In an aspect, a mutation reduces inhibits or eliminates the expression or activity of a gene product. In another aspect a mutation increases, elevates, or augments the activity or expression of a gene product.

In particular, the term “mutation” refers to a variation in the amino acid sequence compared to the sequences shown in Table 1, Table 2 or Table 3, which reduces the expression or function of the protein.

In a preferred embodiment, each copy of a nucleic acid sequence shown in Table 1, Table 2 or Table 3 or a sequence which has at least 80% (at least 85%, at least 90%, at least 95%, at least 96%, at least 97%, at least 98%, at least 99%) sequence identity thereto which is present in the plant is mutated as defined herein (e.g. each genomic copy of a gene encoding said protein in a plant is mutated). For example, each copy of the gene in the allotetraploid genome of N. tabacum may be mutated.

In a preferred embodiment the plant or plant cell according to the present invention is homozygous for the mutation.

In one embodiment preferably the plant or plant cell according to the present invention expresses only the mutated nucleic acid. In other words, in some embodiments no endogenous (or endogenous and functional) protein is present in the plants according to the present invention. In other words if any endogenous protein is present it is preferably in an inactive and/or truncated form.

The mutation may interrupt the nucleic acid sequence which encodes a protein as detailed herein. The interruption may cause the nucleic acid sequence to not be transcribed and/or translated.

The nucleic acid sequence may be interrupted, for example, by deleting or otherwise modifying the ATG start codon of the nucleic acid sequence such that translation of the protein is reduced or prevented.

The nucleic acid sequence may comprise one or more nucleotide change(s) that reduce or prevent expression of the protein or affect protein trafficking. For example, expression of the protein may be reduced or prevented by introduction of one or more pre-mature stop codons, a frame shift, a splice mutant or a non-tolerated amino acid substitution in the open reading frame. A premature stop codon refers to a mutation which introduces a stop codon into the open reading frame and prevents translation of the entire amino acid sequence. The premature stop codon may be a TAG (“amber”), TAA (“ochre”), or TGA (“opal” or “umber”) codon.

Suitably, the premature stop codon may be introduced to Nitab4.5_0003090 g0030.1 (ERF199); as shown in any of SEQ ID No. 5-7.

Suitably, the premature stop codon in Nitab4.5_0003090 g0030.1 (ERF199); as shown in any of SEQ ID No. 5-7 may be a TGA (“opal” or “umber”) premature stop codon.

A frame-shift mutation (also called a framing error or a reading frame shift) is a mutation caused by indels (insertions or deletions) of a number of nucleotides in a nucleic acid sequence that is not divisible by three. Due to the triplet nature of gene expression by codons, the insertion or deletion can change the reading frame, resulting in a completely different translation from the original. A frameshift mutation will often cause the reading of the codons after the mutation to code for different amino acids. The frameshift mutation will commonly result in the introduction of a premature stop codon.

A splice mutant inserts, deletes or changes a number of nucleotides in the specific site at which splicing takes place during the processing of precursor messenger RNA into mature messenger RNA. The deletion of the splicing site results in one or more introns remaining in mature mRNA and may lead to the production of abnormal proteins.

A non-tolerated amino acid substitution refers to a mutation which causes a non-synonymous amino acid substitution in the protein which results in reduced or ablated function of the protein.

Any method known in the art for providing a mutation in a nucleic acid sequence may be used in the present method. For example, homologous recombination may be used, in which a vector is created in which the relevant nucleic acid sequence(s) are mutated and used to transform plants or plant cells. Recombinant plants or plant cells expressing the mutated sequence may then be selected.

The nucleic acid sequence may be wholly or partially deleted. The deletion may be continuous, or may comprise a plurality of sections of sequence. The deletion preferably removes a sufficient amount of nucleotide sequence such that the nucleic acid sequence no longer encodes a functional protein. The deletion may, for example, remove at least 50, 60, 70, 80 or 90% of the coding portion of the nucleic acid sequence.

The deletion may be total, in which case 100% of the coding portion of the nucleic acid sequence is absent, when compared to the corresponding genome a comparable unmodified plant.

Methods for deletion of nucleic acid sequences in plants are known in the art. For example, homologous recombination may be used, in which a vector is created in which the relevant nucleic acid sequence(s) are missing and used to transform plants or plant cells. Recombinant plants or plant cells expressing the new portion of sequence may then be selected.

Plant cells transformed with a vector as described above may be grown and maintained in accordance with well-known tissue culturing methods such as by culturing the cells in a suitable culture medium supplied with the necessary growth factors such as amino acids, plant hormones, vitamins, etc.

Modification of the nucleic acid sequence may be performed using targeted mutagenesis methods (also referred to as targeted nucleotide exchange (TNE) or oligo-directed mutagenesis (ODM)). Targeted mutagenesis methods include, without limitation, those employing zinc finger nucleases, TALENs (see WO2011/072246 and WO2010/079430), Cas9-like, Cas9/crRNA/tracrRNA or Cas9/gRNA CRISPR systems (see WO 2014/071006 and WO2014/093622), meganucleases (see WO2007/047859 and WO2009/059195), or targeted mutagenesis methods employing mutagenic oligonucleotides, possibly containing chemically modified nucleotides for enhancing mutagenesis with sequence complementarity to the gene, into plant protoplasts (e.g., KeyBase® or TALENs).

Alternatively, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines which comprise a gene encoding a protein having a mutation. TILLING uses traditional chemical mutagenesis (e.g. ethyl methanesulfonate (EMS) mutagenesis) followed by high-throughput screening for mutations. Thus, plants, seeds and tissues comprising a gene having the desired mutation may be obtained.

The method may comprise the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such modified plants. Seeds may, for example, be radiated or chemically treated and the plants may be screened for a modified phenotype.

Modified plants may be distinguished from non-modified plants, i.e., wild type plants, by molecular methods, such as the mutation(s) present in the DNA, and by the modified phenotypic characteristics. The modified plants may be homozygous or heterozygous for the mutation.

Suitably the method may comprise transforming a cell of a plant (e.g. a tobacco plant) with a genetic construct which is capable of inhibiting the activity or expression of at least one Nic3 gene (or a construct which is capable of inhibiting the activity or expression of at least one Nic1 ERF gene and/or at least one Nic2 ERF gene, in combination with at least one Nic3 gene).

In some embodiments a modification which increases the activity or expression of at least one Nic3 gene (or of at least one Nic3 gene and at least one Nic1 ERF gene and/or at least one Nic2 ERF gene in combination) and thereby increases alkaloid content is selected from the group consisting of:

increasing, promoting or augmenting transcription, translation or expression of the at least one Nic3 gene (or the at least one Nic3 gene and the at least one Nic1 ERF gene and/or the at least one Nic2 ERF gene in combination);

increasing synthesis of the polypeptide encoded by at least one Nic3 gene (or of the at least one Nic3 gene and the at least one Nic1 ERF gene and/or the at least one Nic2 ERF gene in combination), or its release from intracellular stores; or

decreasing the rate of degradation of the polypeptide encoded by at least one Nic3 gene (or of the at least one Nic3 gene and the at least one Nic1 ERF gene and/or the at least one Nic2 ERF gene in combination).

Suitably the method may comprise transforming a cell of a plant (e.g. a tobacco plant) with a genetic construct which encodes at least one exogenous Nic3 gene (or which encodes at least one Nic3 gene and at least one Nic1 ERF gene and/or at least one Nic2 ERF gene in combination), or which comprises a nucleotide sequence which encodes a protein which is capable of promoting or augmenting at least one endogenous Nic3 gene (or at least one endogenous Nic3 gene and at least one endogenous Nic1 ERF gene and/or at least one endogenous Nic2 ERF gene in combination). It will be appreciated that each of these options would result in an increased activity and expression of the polypeptide encoded by the at least one Nic3 gene (or of the at least one Nic3 gene and at least one Nic1 ERF gene and/or at least one Nic2 ERF gene in combination). The method may comprise regenerating the plant from the transformed cell.

Thus, there is provided use of genetic construct which is capable of increasing the activity and/or expression of a polypeptide encoded by at least one Nic3 (or at least one Nic3 gene and at least one Nic1 ERF gene and/or at least one Nic2 ERF gene in combination), for increasing the alkaloid content in a plant transformed with the construct.

The genetic construct may encode a polypeptide comprising the amino acid sequence as set out in: Table 1, Table 2 and/or Table 3, or a functional variant or functional fragment or orthologue thereof.

In some embodiments a method or use according to the present invention comprises increasing the alkaloid content of a plant (e.g. a tobacco plant) or cell by increasing the activity or expression of a Nic3 gene, or the activity or expression of a Nic3 gene and a Nic1 ERF gene and/or a Nic2 ERF gene.

The term “inhibiting” (e.g. inhibiting the activity or expression of a Nic3 gene) as used herein means that the activity or expression of the gene (e.g. Nic3 gene) is lower or decreased compared with the gene activity or expression of the gene in a comparable product or the amount or activity of a protein produced by the gene is lower.

In one embodiment the term “inhibiting” (e.g. inhibiting the activity or expression of a Nic3 gene) as used herein means that the activity or expression of the Nic3 gene is lower compared with the gene activity or expression of the gene in a comparable product.

The activity of specific Nic3 gene, Nic1 ERF gene or Nic2 ERF gene can be measured by measuring transcription of the gene. Methods for measuring transcription are well known in the art and include, amongst others, northern blot, RNA-Seq, in situ hybridization, DNA microarrays and RT-PCR. Alternatively, the activity of a gene may be measured indirectly by measuring the level of the gene product for example the protein encoded by said gene.

In some embodiments the activity or expression of a Nic3 gene, Nic1 ERF gene or Nic2 ERF gene may be modulated i.e. increased or decreased by at least about 10% 20% 30%, or 40%, suitably at least about 50%, 60%, 70%, more suitably at least about 80%, 90%, 95% or 100% when compared to the activity or expression of said gene in a plant (e.g. a tobacco plant) which has not been modified in accordance with the present invention.

Suitably, the expression or function of the Nic3 gene, Nic1 ERF gene or Nic2 ERF gene may be reduced, partly inactivated, inhibited, eliminated, knocked out or lost such that the protein expression or function of said gene is not detectable.

In one aspect, the at least one Nic3 gene, Nic1 ERF gene or Nic2 ERF gene is knocked out. In other words, the gene has been rendered completely inoperative.

In a preferred embodiment the Nic3 gene may have substantially no activity or expression, which means that the plant may comprise less than about 1% (suitably less than about 0.1%) activity or expression, preferably when compared to a plant which has not been modified to inhibit the activity or expression of a Nic3 gene.

In a preferred embodiment the Nic1 ERF gene may have substantially no activity or expression, which means that the plant may comprise less than about 1% (suitably less than about 0.1%) activity or expression, preferably when compared to a plant which has not been modified to inhibit the activity or expression of a Nic1 ERF gene.

In a preferred embodiment the Nic2 ERF gene may have substantially no activity or expression, which means that the plant may comprise less than about 1% (suitably less than about 0.1%) activity or expression, preferably when compared to a plant which has not been modified to inhibit the activity or expression of a Nic2 ERF gene.

An “ERF gene” as used herein refers to a transcription factor gene which belongs to the ethylene response factor (ERF) subfamily.

A “Nic1 ERF gene” as used herein refers to an ERF gene which the present inventors have identified in WO2018/237107 as mapping to the Nic1 region. Nic1 ERF genes as used herein are listed in Table 1 along with their corresponding nucleotide, cDNA, cds and amino acid sequence identifiers.

Suitably the at least one Nic1 ERF gene for use in the present invention is any one of those listed in Table 1.

The genomic sequences of each of the Nic1 ERFs and the Nic2 ERFs listed in the tables above are identical to their corresponding coding sequences with the exception of the Nic1 ERF ERF17L3. The genomic sequence of ERF17L3 (SEQ ID No. 1) is not identical to the coding sequence of ERF17L3 (SEQ ID No. 3).

A “Nic2 ERF gene” as used herein refers to an ERF gene which t maps to the Nic2 region. Nic2 ERF genes as used herein are listed in Table 2 below along with their corresponding nucleotide, cDNA, cds and amino acid sequence identifiers.

Suitably the Nic2 ERF gene for use in the present invention is any one of those listed in Table 2. In one embodiment the at least one Nic3 gene referred to herein may be encoded by a polynucleotide sequence shown in Table 3.

Suitably, the at least one Nic3 gene referred to herein may be encoded by a polynucleotide sequence comprising:
i) a polynucleotide sequence shown herein as SEQ ID No. 73, SEQ ID No. 76, SEQ ID No. 79, SEQ ID No. 82, SEQ ID No. 85, SEQ ID No. 88, SEQ ID No. 91, SEQ ID No. 94, SEQ ID No. 97, SEQ ID No. 100, SEQ ID No. 103, SEQ ID No. 106, SEQ ID No. 109, SEQ ID No. 112, SEQ ID No. 115, SEQ ID No. 118, SEQ ID No. 121, SEQ ID No. 124, SEQ ID No. 127, SEQ ID No. 130, SEQ ID No. 133, SEQ ID No. 136, SEQ ID No. 139, SEQ ID No. 142, SEQ ID No. 145, SEQ ID No. 148 or SEQ ID No. 151 (suitably SEQ ID No. 73, 118, 124 or 127); or a sequence which has at least 80% identity thereto; or
ii) a functional fragment of the polynucleotide sequence shown in i) which functional fragment encodes a Nic3 gene, or
iii) a polynucleotide which encodes a polypeptide comprising the amino acid sequence shown herein as SEQ ID No. 75, SEQ ID No. 78, SEQ ID No. 81, SEQ ID No. 84, SEQ ID No. 87, SEQ ID No. 90, SEQ ID No. 93, SEQ ID No. 96, SEQ ID No. 99, SEQ ID No. 102, SEQ ID No. 105, SEQ ID No. 108, SEQ ID No. 111, SEQ ID No. 114, SEQ ID No. 117, SEQ ID No. 120, SEQ ID No. 123, SEQ ID No. 126, SEQ ID No. 129, SEQ ID No. 132, SEQ ID No. 135, SEQ ID No. 138, SEQ ID No. 141, SEQ ID No. 144, SEQ ID No. 147, SEQ ID No. 150 or SEQ ID No. 153, suitably SEQ No. 75, 120, 126 or 129), or
iv) a polynucleotide sequence which can hybridize to the polynucleotide taught in i), ii) or iii) above under high stringency conditions, or
v) a polynucleotide sequence which has at least 80% (preferably 85%, preferably 90%, preferably 95%, more preferably 96%, more preferably 97%, more preferably 98%) identity with the polynucleotide shown in i), ii) or iii) above, or
vi) a polynucleotide sequence which differs from polynucleotide shown in i), ii) or iii) due to degeneracy of the genetic code.

In one embodiment the at least one Nic1 ERF gene referred to herein may be encoded by a polynucleotide sequence comprising:
i) a polynucleotide sequence shown herein as SEQ ID No. 1, SEQ ID No. 3; SEQ ID No. 5, SEQ ID No. 9, SEQ ID No. 13, SEQ ID No. 17, SEQ ID No. 21, SEQ ID No. 25, SEQ ID No. 29 or SEQ ID No. 33; or
ii) a functional fragment of the polynucleotide sequence shown in i) which functional fragment encodes a Nic1 ERF synthesis gene, or
iii) a polynucleotide which encodes a polypeptide comprising the amino acid sequence shown herein as SEQ ID No. 4, SEQ ID No. 8, SEQ ID No. 12, SEQ ID No. 16, SEQ ID No. 20, SEQ ID No. 24, SEQ ID No. 28, SEQ ID No. 32 or SEQ ID No. 36, or
iv) a polynucleotide sequence which can hybridize to the polynucleotide taught in i), ii) or iii) above under high stringency conditions, or
v) a polynucleotide sequence which has at least 70% (preferably 80%, preferably 85%, preferably 90%, preferably 95%, more preferably 96%, more preferably 97%, more preferably 98%) identity with the polynucleotide shown in i), ii) or iii) above, or
vi) a polynucleotide sequence which differs from polynucleotide shown in i), ii) or iii) due to degeneracy of the genetic code.

In one embodiment the at least one Nic2 ERF gene referred to herein may be encoded by a polynucleotide sequence comprising:
i) a polynucleotide sequence shown herein as SEQ ID No. 37, SEQ ID No. 41, SEQ ID No. 45, SEQ ID No. 49, SEQ ID No. 53, SEQ ID No. 57, SEQ ID No. 61, SEQ ID No. 65 or SEQ ID No. 69; or
ii) a functional fragment of the polynucleotide sequence shown in i) which functional fragment encodes a Nic1 ERF gene, or
iii) a polynucleotide which encodes a polypeptide comprising the amino acid sequence shown herein as SEQ ID No. 40, SEQ ID No. 44, SEQ ID No. 48, SEQ ID No. 52, SEQ ID No. 56, SEQ ID No. 60, SEQ ID No. 64, SEQ ID No. 68 or SEQ ID No. 72, or iv) a polynucleotide sequence which can hybridize to the polynucleotide taught in i), ii) or iii) above under high stringency conditions, or
v) a polynucleotide sequence which has at least 70% (preferably 80%, preferably 85%, preferably 90%, preferably 95%, more preferably 96%, more preferably 97%, more preferably 98%) identity with the polynucleotide shown in i), ii) or iii) above, or
vi) a polynucleotide sequence which differs from polynucleotide shown in i), ii) or iii) due to degeneracy of the genetic code.

In one embodiment the at least one Nic3 gene for use in accordance with the present invention may be endogenous to the plant (e.g. a tobacco plant).

In one embodiment the at least one Nic1 ERF gene for use in accordance with the present invention may be endogenous to the plant (e.g. a tobacco plant).

In one embodiment the at least one Nic2 ERF gene for use in accordance with the present invention may be endogenous to the plant (e.g. a tobacco plant).

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene) or a plant cell. For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

In another embodiment the at least one Nic3 gene for use in accordance with the present invention may be exogenous to the plant (e.g. a tobacco plant).

In another embodiment the at least one Nic1 ERF gene for use in accordance with the present invention may be exogenous to the plant (e.g. a tobacco plant).

In another embodiment the at least one Nic2 ERF gene for use in accordance with the present invention may be exogenous to the plant (e.g. a tobacco plant).

The term “exogenous gene” can mean the gene that is transformed into the unmodified plant is from an external source, i.e. from a different species to the one being transformed. The exogenous gene may comprise a nucleic acid sequence substantially the same or different to an endogenous gene in the unmodified plant. The exogenous gene may be derived from a genomic or cDNA sequence corresponding to the gene from any species. The exogenous gene may form a chimeric gene. The exogenous gene may encode a polypeptide comprising the amino acid sequence as set out in Table 1, or a functional variant or fragment or orthologue thereof. The exogenous gene may comprise the nucleotide sequence as set out in Table 2, or a functional variant or fragment or orthologue thereof. The exogenous gene may comprise the nucleotide sequence as set out in Table 3, or a functional variant or fragment or orthologue thereof.

The present invention also provides the use of a Nic3 gene for modulating the alkaloid content of a plant.

In one embodiment the invention further provides the use of a Nic3 gene, and optionally a Nic1 ERF and/or a Nic2 ERF for modulating the alkaloid content of a plant.

Methods for decreasing expression of genes or gene products are well documented in the art. Any method described herein for modulating activity or expression of a Nic3 gene may be used to modify the activity or expression of a Nic3 gene, and optionally a Nic1 ERF gene and/or a Nic2 ERF gene.

In one embodiment the activity or expression of a Nic3 gene or the activity or expression of a Nic3 gene, and optionally a Nic1 ERF gene and/or a Nic2 ERF gene may be inhibited by any method known in the art.

Methods for inhibiting the activity or expression of a Nic3 gene or the activity or expression of a Nic3 gene and a Nic1 ERF gene and/or a Nic2 ERF gene may include gene editing, targeted mutagenesis, RNA interference, antisense or sense co-suppression (see Wang and Wagner 2003, Planta Volume 216, Issue 4, pp 686-691, which is incorporated herein by reference). In one embodiment the inhibition of activity or expression of a gene may be achieved by the use of gene editing. Gene editing may be carried out using any method known in the art. A few non-limiting examples are presented herein.

In one embodiment the inhibition of activity or expression of a Nic3 gene or the activity or expression of a Nic3 gene and a Nic1 ERF gene and/or a Nic2 ERF gene may be achieved using gene editing methods including CRISPR, including use of the CRISPR/Cas9 system.

CRISPR/Cas9 genomic editing tools are available commercially such as “Guide-it” from Clontech (Avenue du President Kennedy 78100 Saint-Germain-en-Laye, France).

Suitably, to generate a gene editing vector pRGEB-M24, the rice snoRNA U3 promoter in the vector pRGEB31 may be substituted with the M24 promoter amplified from pSiM24 (Sahoo et al., 2014 incorporated herein by reference) through infusion cloning assisted with HindIII and BsaI as described in WO2018/237107 which is incorporate herein by reference. For example, one pair of oligos can be designed to specifically target each of the candidate genes.

The oligo pairs are first annealed to produce a double-stranded fragment with 4-nt 5′ overhangs at both ends, and then ligated into the BsaI digested pRGEB-M24 vector.

Another method of gene editing includes the use of TALEN (transcription activator-like effector nuclease) technology with kits available commercially (e.g. from Addgene, 1Kendall Sq. Ste. B7102, Cambridge, Mass. 02139, USA). In one embodiment the inhibition of activity or expression of the at least one Nic3 gene or the activity or expression of at least one Nic3 gene and at least one Nic1 ERF gene and/or at least one Nic2 ERF gene may be achieved using TALEN.

In another embodiment the method may comprise the use of Zinc Finger Nucleases such as the CompoZr® Zinc Finger Nuclease Technology available from Sigma-Aldrich. Another embodiment may comprise the use of meganucleases (or a further method) described in Silva et al. Curr Gene Ther. February 2011; 11(1): 11-27 (the teaching of which is incorporated herein by reference). In one embodiment the method for inhibiting the activity or expression of a Nic3 gene or a Nic3 gene and a Nic1 ERF gene and/or a Nic2 ERF gene may be targeted mutagenesis. Any method of targeted mutagenesis may be used. In one embodiment the method may be oligonucleotide-directed mutagenesis (ODM) such as KeyBase® available from Keygene (Agro Business Park 90, 6708 P W Wageningen, The Netherlands). In another embodiment, inhibition of the activity or expression of a Nic3 gene or the activity or expression of a Nic3 gene and a Nic1 ERF gene and/or a Nic2 ERF gene may be achieved by use of a construct or vector (e.g. a plasmid).

Genetic constructs of the invention may be in the form of an expression cassette, which may be suitable for inhibition of the activity or expression of a Nic3 gene or the activity or expression of a Nic3 gene and a Nic1 ERF gene and/or a Nic2 ERF gene in a host cell or for increasing the activity or expression of a Nic3 gene or the activity or expression of a Nic3 gene and a Nic1 ERF gene and/or a Nic2 ERF gene in a host cell. The genetic construct may be introduced into a host cell without it being incorporated in a vector. For instance, genetic construct, which may be a nucleic acid molecule, may be incorporated within a liposome or a virus particle. Alternatively, a purified nucleic acid molecule (e.g. histone-free DNA or naked DNA) may be inserted directly into a host cell by suitable means, e.g. direct endocytotic uptake. The genetic construct may be introduced directly into cells of a host subject (e.g. a plant) by transfection, infection, microinjection, cell fusion, protoplast fusion or ballistic bombardment. Alternatively, genetic constructs of the invention may be introduced directly into a host cell using a particle gun.

Alternatively, the genetic construct may comprise or be harboured within a recombinant vector, for expression in a suitable host cell. The recombinant vector may be a plasmid, cosmid or phage.

Such recombinant vectors are highly useful for transforming host cells with the genetic construct of the invention, and for replicating the expression cassette therein. The skilled technician will appreciate that genetic constructs of the invention may be combined with many types of backbone vector for expression purposes. The backbone vector may be a binary vector, for example one which can replicate in both E. coli and Agrobacterium tumefaciens. For example, a suitable vector may be a pBIN plasmid, such as pBIN19 (Bevan M., 1984, Nucleic Acids Research 12:8711-21). Recombinant vectors may include a variety of other functional elements in addition to the sequence which inhibits the activity or expression of the at least one Nic3 gene, or the activity or expression of at least one Nic3 gene and at least one Nic1 ERF gene and/or at least one Nic2 ERF gene. For example, the vector may comprise a promoter. In addition, the recombinant vector may be designed such that it autonomously replicates in the cytosol of the host cell. In this case, elements which induce or regulate DNA replication may be required in the recombinant vector.

Alternatively, the recombinant vector may be designed such that it integrates into the genome of a host cell. In this case, DNA sequences which favor targeted integration (e.g. by homologous recombination) are envisaged.

The recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. The vector may also comprise DNA involved with regulating expression of the coding sequence, or for targeting the expressed polypeptide to a certain part of the host cell, e.g. to trichomes or glandular trichomes. Hence, the vector may comprise at least one additional element selected from a group consisting of: a selectable marker gene (e.g. an antibiotic resistance gene); a polypeptide termination signal; and a protein targeting sequence (e.g. a transit peptide).

In one embodiment, the method or use may comprise inhibiting the activity or expression of a Nic3 gene or the activity or expression of a Nic3 gene and a Nic1 ERF gene and/or a Nic2 ERF gene using an interfering oligonucleotide. In one embodiment the oligonucleotide is RNA based.

In one embodiment the oligonucleotide is RNA interference (RNAi), e.g. dsRNAi. In one embodiment the method may comprise transforming a cell of a plant (e.g. a tobacco plant) with an RNAi molecule, e.g. dsRNAi, which inhibits the activity or expression of a Nic3 gene, or the activity or expression of a Nic3 gene and a Nic1 ERF gene and/or a Nic2 ERF gene. Suitably the RNAi molecule may be provided from a vector which may be introduced into a cell of the plant, e.g. virus-included gene silencing may be used which carries a fragment of a relevant gene (for example a fragment which is from 100 to 300 nucleotides in length) and produces dsRNA to trigger RNA-mediated gene silencing.

In one embodiment the activity or expression of at least one Nic3 gene, Nic1 ERF gene, and/or Nic2 ERF gene is decreased by at least 30%, 35%, 40%, 45%, 50%, 55%, 60%, 65%, 70%, 75%, 80%, 85%, 90%, 95% or more, or by 100% compared to the activity or expression of the polypeptide in a comparable plant or part thereof or cell.

In one embodiment the activity or expression of the at least one Nic3 gene, the at least one Nic1 ERF gene and the at least one Nic2 ERF gene is decreased by at least 30%, 35%, 40%, 45%, 50%, 55%, 60%, 65%, 70%, 75%, 80%, 85%, 90%, 95% or more, or by 100% compared to the activity or expression of the polypeptide in the wild-type plant or a comparable plant, or part thereof or cell.

The activity or expression of the at least one Nic3 gene or the activity or expression of at least one Nic3 gene and at least one Nic1 ERF gene and/or at least one Nic2 ERF gene may be inhibited by any method known in the art. In any of the preceding embodiments the activity or expression of the at least one Nic3 gene or the activity or expression of at least one Nic3 gene and at least one Nic1 ERF gene and/or at least one Nic2 ERF gene may be inhibited by any method including gene editing methods including CRISPR, including use of the CRISPR-Cas9 system, RNA interference (RNAi), antisense or sense co-suppression, gene editing or targeted mutagenesis. In any of the preceding embodiments the activity or expression of at least one Nic3 gene or the activity or expression of at least one Nic3 gene and at least one Nic1 ERF gene and/or at least one Nic2 ERF gene may be inhibited using an RNAi method for example using miRNA, siRNA, dsRNA or shRNA.

In one embodiment the construct which modulates Nic3 gene activity or expression or the activity or expression of at least one Nic3 gene and at least one Nic1 ERF gene and/or at least one Nic2 ERF gene may be comprised in a vector. Suitably the vector may be a plasmid.

In one embodiment, the vector for use in the present invention is the Agrobacterium-based plasmid.

Accordingly in one embodiment plants (e.g. a tobacco plants) and plant propagation materials (e.g. a tobacco plant propagation materials), leaves (e.g. tobacco leaves), cut harvested leaves, processed leaves (e.g. processed tobacco leaves) or cut and processed leaves (e.g. cut and processed tobacco leaves) are provided wherein expression of a Nic3 gene or the activity or expression of a Nic3 gene and a Nic1 ERF gene and/or a Nic2 ERF gene is modulated.

In another embodiment the cell (e.g. tobacco cell), plant (e.g. a tobacco plant) or part thereof, and/or plant propagation material may comprise a construct which modulates the activity or expression of a Nic3 gene or the activity or expression of a Nic3 gene and a Nic1 ERF gene and/or a Nic2 ERF gene. In one embodiment the construct decreases the activity or expression of a Nic3 gene or the activity or expression of a Nic3 gene and a Nic1 ERF gene and/or a Nic2 ERF gene. In another embodiment the construct increases the activity or expression of a Nic3 gene or the activity or expression of a Nic3 gene and a Nic1 ERF gene and/or a Nic2 ERF gene.

In a further embodiment the cell (e.g. tobacco cell), plant (e.g. a tobacco plant) or part thereof and/or plant propagation material according to the invention may comprise:
i) a polynucleotide sequence shown in Table 3; or
ii) a functional fragment of the polynucleotide sequence shown in i) which functional fragment encodes a Nic3 gene, or
iii) a polynucleotide which encodes a polypeptide comprising the amino acid sequence shown in Table 3, or
iv) a polynucleotide sequence which can hybridize to the polynucleotide taught in i), ii) or iii) above under high stringency conditions, or
v) a polynucleotide sequence which has at least 80% (preferably 85%, preferably 90%, preferably 95%, more preferably 96%, more preferably 97%, more preferably 98%) identity with the polynucleotide shown in i), ii) or iii) above, or
vi) a polynucleotide sequence which differs from polynucleotide shown in i), ii) or iii) due to degeneracy of the genetic code. Suitably the Table 3 sequences are as described hereinbefore.

In a further embodiment the cell (e.g. tobacco cell), plant (e.g. a tobacco plant) or part thereof and/or plant propagation material according to the invention may comprise:
i) a polynucleotide sequence selected from Table 3, a polynucleotide sequence selected from Table 1, and a polynucleotide sequence selected from Table 2; or
ii) a functional fragment of the polynucleotide sequence shown in i) which functional fragment encodes a Nic3 gene, Nic1 ERF gene or Nic2 ERF gene; or
iii) a polynucleotide which encodes a polypeptide comprising the amino acid sequence shown in Table 3, Table 1 and Table 2; or
iv) a polynucleotide sequence which can hybridize to the polynucleotide taught in i), ii) or iii) above under high stringency conditions; or
v) a polynucleotide sequence which has at least 80% (preferably 85%, preferably 90%, preferably 95%, more preferably 96%, more preferably 97%, more preferably 98%) identity with the polynucleotide shown in i), ii) or iii) above; or
vi) a polynucleotide sequence which differs from polynucleotide shown in i), ii) or iii) due to degeneracy of the genetic code. Suitably the Table 1, 2 or 3 sequences are as described hereinbefore.

In one embodiment the cell (e.g. tobacco cell) is grown in a cell culture.

In one embodiment, at least one Nic3 gene (or at least one Nic3 gene and at least one Nic1 ERF gene and/or at least one Nic2 ERF gene) is used to modulate alkaloid content (e.g. nicotine content) in a cell or cell culture (e.g. a tobacco cell culture).

In an advantageous embodiment, inhibition of the activity or expression of at least one Nic3 gene or the activity or expression of at least one Nic3 gene and at least one Nic1 ERF gene and/or at least one Nic2 ERF gene may result in a decrease in alkaloid content. Suitably inhibition of the activity or expression of at least one Nic3 gene or the activity or expression of at least one Nic3 gene and at least one Nic1 ERF gene and/or at least one Nic2 ERF gene may result in a decrease in nicotine content.

In another embodiment increasing the activity or expression of a Nic3 gene (or the activity or expression of a Nic3 gene and a Nic1 ERF gene and/or a Nic2 ERF gene) may result in a decrease in alkaloid content. Suitably increasing the activity or expression of a Nic3 ERF or the activity or expression of a Nic3 gene and a Nic1 ERF gene and/or a Nic2 ERF gene may result in a decrease in nicotine content.

In one embodiment the plant or part thereof is a tobacco plant. In one embodiment the tobacco plant or part thereof according to the present invention is a Burley or Flue-cured plant modified in Burley or Flue-cured plant modified in accordance with the present invention. In one embodiment the tobacco plant (e.g. modified tobacco plant) according to the present invention is an Oriental or Turkish tobacco plant.

In one embodiment the tobacco plant or part thereof is cured. In one embodiment the tobacco plant or part thereof is cured e.g. air-cured, flue-cured, fire-cured or sun-cured. In a further aspect, the tobacco plant or part thereof is flue-cured. In a further aspect, the tobacco plant or part thereof is air-cured.

Flue-curing is well-known in the art and refers to the process of curing tobacco with flues which are fed by fire boxes or gas fueled systems. This process heat-cures the tobacco without exposing it to smoke, slowly raising the temperature over the course of the curing. This method produces tobacco that is high in sugar and has medium to high levels of nicotine. The Smith Tobacco Barn is an example of a traditional, flue-cured tobacco barn.

Air-cured tobaccos include Burley, Maryland, and dark tobaccos. The common factor is that curing is primarily without artificial sources of heat and humidity. Burley tobaccos are light to dark brown in colour, high in oil, and low in sugar. Burley tobaccos are air-cured in barns. Major Burley growing countries are Argentina, Brazil, Italy, Malawi, and the U.S. Burley tobacco plants include, for example, Clay 402, Clay 403, Clay 502, Ky 14, Ky 907, Ky 910, Ky 8959, NC 2, NC 3, NC 4, NC 5, NC 2000, TN 86, TN 90, TN 97, R 610, R 630, R711, R 712, NCBH 129, Bu 21xKy 10, HBO4P, Ky 14xL 8, Kt 200, Newton 98, Pedigo 561, Pf561 and Va 509.

Maryland tobaccos have good burning properties, low nicotine and a neutral aroma. Major Maryland growing countries include the U.S. and Italy. Dark air-cured tobaccos are distinguished from other types primarily by its fermentation process which gives dark air-cured tobacco its medium- to dark-brown colour and distinct aroma. Their leaves have low sugar content but high nicotine content. Dark air-cured tobaccos are mainly used in the production of chewing tobacco and snuff. Major growing regions for dark fire-cured tobaccos are Tennessee, Kentucky, and Virginia, USA.

The term “functional fragment” as used herein refers to a portion of a polynucleotide that is capable of functioning in the same way as the polynucleotide. For example, if the polynucleotide is an ERF gene then the functional fragment must be capable of functioning as an ERF gene, e.g. the functional fragment retains the activity of the ERF gene. The functional fragment may have a level of activity which is equal to or greater than the level of activity of a full length polynucleotide. In one embodiment a functional fragment may be a portion of a Nic3 gene as discussed herein comprising at least 50, 75, 100, 150, 200, 300, 400, 500, 600, 700, 800, 900 or 1000 contiguous nucleotides. Suitably the functional fragments comprises a domain of the Nic3 gene with SEQ ID No. 75, 120, 127 or 129 as described hereinbefore. In some embodiments the functional fragment may comprise at least 150 nucleotides of a Nic1 ERF discussed herein.

In one embodiment a functional fragment may be a portion of a Nic1 ERF gene as discussed herein comprising at least 50, 75, 100, 150, 200, 300, 400, 500, 600, 700, 800, 900 or 1000 contiguous nucleotides. In some embodiments the functional fragment may comprise at least 150 nucleotides of a Nic1 ERF discussed herein.

In one embodiment a functional fragment of a Nic2 ERF gene may be a portion of a Nic2 ERF gene as discussed herein comprising at least 50, 75, 100, 150, 200, 300, 400, 500, 600, 700, 800, 900 or 1000 contiguous nucleotides. In some embodiments the functional fragment may comprise at least 150 nucleotides of a Nic2 ERF discussed herein.

The term “functional variant” as used herein refers to variability which may arise in genomic sequences without significant loss of activity in either the gene function and/or the protein function. For example some amino acids present in a polypeptide (or some nucleotides present in a polynucleotide) may be substituted without significant loss of activity. The functional variant may have a level of activity which is equal to, or greater than, the level of activity of the non-variant polynucleotide and/or polypeptide. Sequences which differ from the genes disclosed herein due to degeneracy of the genetic code are functional variants. A variant may differ from the sequence of interest by as few as 10, as few as 9, as few as 8, as few as 7 as few as 6, as few as 5, as few as 4, as few as 3, as few as 2 or as few as 1 amino acid(s).

The term “degeneracy of the genetic code” as used herein refers to the redundancy in codons encoding polypeptide sequences exhibited as the multiplicity of three-codon combinations specifying an amino acid. For example in an mRNA molecule encoding a polypeptide having an isoleucine amino acid, isoleucine can be encoded by AUU, AUC or AUA. This means that a DNA molecule encoding the RNA can have multiple sequences yet the resulting polypeptide will have the same sequence. In other words polymorphic nucleotide sequences can encode the same polypeptide product. This means that one nucleic acid sequence can comprise a sequence with very low sequence identity to a second sequence while encoding the same polypeptide sequence.

Sequences having a degree of sequence identity or sequence homology with amino acid sequence(s) of a polypeptide having the specific properties described herein or of any nucleotide sequence described herein may be functional variants.

The term “orthologue” as used herein refers to genes which are derived from a common ancestral gene and which are found in different species as a result of speciation. Orthologues may share at least 70%, 75%, 80%, 85%, 90%, 91%, 92%, 93%, 94%, 95%, 96%, 97%, 98%, 99% or greater sequence identity at the nucleotide sequence and or amino acid sequence level. Orthologous genes often share the same or similar functions i.e. have conserved function.

In some embodiments of the present invention a promoter may be provided. The promoter for use in the present invention may be one or more selected from the group consisting of: a constitutive promoter, a senescence-specific promoter, a tissue-specific promoter, a developmentally-regulated promoter and an inducible promoter. In one embodiment the promoter may be a constitutive promoter.

A constitutive promoter directs the expression of a gene throughout the various parts of a plant continuously during plant development, although the gene may not be expressed at the same level in all cell types. Examples of known constitutive promoters include those associated with the cauliflower mosaic virus 35S transcript (Odell J T, Nagy F, Chua N H (1985) Identification of DNA sequences required for activity of the cauliflower mosaic virus 35S promoter, Nature 313 810-2), the rice actin 1 gene (Zhang W, McElroy D, Wu R. (1991) Analysis of rice Act1 5′ region activity in transgenic rice plants (Plant Cell 3 1155-65)) and the maize ubiquitin 1 gene (Cornejo M J, Luth D, Blankenship K M, Anderson O D, Blechl A E. (1993). Activity of a maize ubiquitin promoter in transgenic rice. Plant Molec. Biol. 23 567-81) which are incorporated herein by reference. Constitutive promoters include the Carnation Etched Ring Virus (CERV) promoter. The sequence of carnation etched ring virus DNA: comparison with cauliflower mosaic virus and retroviruses ((Hull R, Sadler J, Longstaff M 1986 EMBO Journal, 5(2):3083-3090) which is incorporated herein by reference).

The constitutive promoter may be selected from a: a carnation etched ring virus (CERV) promoter, a cauliflower mosaic virus (CaMV 35S promoter), a promoter from the rice actin 1 gene or the maize ubiquitin 1 gene. Suitably the promoter may be a CERV promoter.

Alternatively in some embodiments the promoter may not be a cauliflower mosaic virus (CaMV 35S promoter). In one embodiment the promoter may be a senescence-specific promoter. A “senescence-specific promoter” (SAG) can be a promoter, which is associated with controlling the expression of a senescence-associated gene. Hence, the promoter can restrict expression of a coding sequence (i.e. a gene) to which it is operably linked substantially exclusively in senescing tissue. Therefore, a senescence-specific promoter can be a promoter capable of preferentially promoting gene expression in a plant tissue in a developmentally-regulated manner such that expression of a 3′ protein-coding region occurs substantially only when the plant tissue is undergoing senescence. It will be appreciated that senescence tends to occur in the older parts of the plant, such as the older leaves, and not in the younger parts of the plants, such as the seeds.

One example of a plant which is known to express numerous senescence-associated genes is Arabidopsis. Hence, the promoter may be isolated from a senescence-associated gene in Arabidopsis. Gepstein et al. (The Plant Journal, 2003, 36, 629-642), incorporated herein by reference, conducted a detailed study of SAGs and their promoters using Arabidopsis as a model.

The genetic construct may comprise a promoter from any of the SAGs disclosed in this paper. For example, a suitable promoter may be selected from a group consisting of SAG12, SAG13, SAG101, SAG21 and SAG18, or a functional variant or a functional fragment thereof.

In one embodiment the promoter may be a SAG12 or a SAG13 promoter. In one embodiment, the promoter may be a SAG12 promoter, which will be known to the skilled technician, or a functional variant or a functional fragment thereof (Gan & Amasino, 1997, Plant Physiology, 113: 313-319, incorporated herein by reference). Suitable promoters and sequences thereof may be found in WO2010/097623 (incorporated herein by reference).

In another embodiment the promoter may be a tissue-specific promoter. A tissue-specific promoter is one which directs the expression of a gene in one (or a few) parts of a plant, usually throughout the lifetime of those plant parts. The category of tissue-specific promoter commonly also includes promoters whose specificity is not absolute, i.e. they may also direct expression at a lower level in tissues other than the preferred tissue. A number of tissue-specific promoters are known in the art and include those associated with the patatin gene expressed in potato tuber and the high molecular weight glutenin gene expressed in wheat, barley or maize endosperm.

Any of these promoters may be used in the present invention.

Suitably the tissue-specific promoter may be a leaf-specific promoter. Suitably leaf-specific promoters may include ASYMMETRIC LEAVES 1 (AS1).

In a particularly preferred embodiment the tissue-specific promoter is a root-specific promoter.

In another embodiment the promoter may be a developmentally-regulated promoter. A developmentally-regulated promoter directs a change in the expression of a gene in one or more parts of a plant at a specific time during plant development. The gene may be expressed in that plant part at other times at a different (usually lower) level, and may also be expressed in other plant parts.

In one embodiment the promoter may be an inducible promoter. An inducible promoter is capable of directing the expression of a gene in response to an inducer. In the absence of the inducer the gene will not be expressed. The inducer may act directly upon the promoter sequence, or may act by counteracting the effect of a repressor molecule. The inducer may be a chemical agent such as a metabolite, a protein, a growth regulator, or a toxic element, a physiological stress such as heat, wounding, or osmotic pressure, or an indirect consequence of the action of a pathogen or pest. A developmentally-regulated promoter might be described as a specific type of inducible promoter responding to an endogenous inducer produced by the plant or to an environmental stimulus at a particular point in the life cycle of the plant. Examples of known inducible promoters include those associated with wound response, such as described by Warner S A, Scott R, Draper J. (1993) (Isolation of an asparagus intracellular PR gene (AoPR1) wound-responsive promoter by the inverse polymerase chain reaction and its characterization in transgenic tobacco. Plant J. 3 191-201), incorporated herein by reference, temperature response as disclosed by Benfey & Chua (1989) (Benfey, P. N., and Chua, N—H. (1989) Regulated genes in transgenic plants. Science 244 174-181), incorporated herein by reference, and chemically induced, as described by Gatz (1995) (Gatz, C. (1995) Novel inducible/repressible gene expression systems. Methods in Cell Biol. 50 411-424), incorporated herein by reference.

Thus in one embodiment the promoter may be selected from the group consisting of: the CERV promoter, the cauliflower mosaic virus 35S promoter (full or truncated), the rubisco promoter, the pea plastocyanin promoter, the nopaline synthase promoter, the chlorophyll r/b binding promoter, the high molecular weight glutenin promoter, the α, β-gliadin promoter, the hordein promoter and the patatin promoter.

In one embodiment the promoter may be the CaMV 35S promoter or a modified 35S promoter with a duplicated enhancer region or double enhancer region (R. Kay et al. Science. 1987 Jun. 5; 236(4806):1299-302 which is incorporated herein by reference).

In one embodiment the promoter may be the native promoter.

As used herein “native promoter” refers to the promoter which is endogenous to the gene i.e. which is operably linked to the gene in nature.

The recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. The vector may also comprise DNA involved with regulating expression of the coding sequence, or for targeting the expressed polypeptide to a certain part of the host cell, e.g. the chloroplast. Hence, the vector may comprise at least one additional element selected from a group consisting of: a selectable marker gene (e.g. an antibiotic resistance gene); a polypeptide termination signal; and a protein targeting sequence (e.g. a chloroplast transit peptide).

Examples of suitable marker genes include antibiotic resistance genes such as those conferring resistance to Kanamycin, Geneticin (G418) and Hygromycin (npt-II, hyg-B); herbicide resistance genes, such as those conferring resistance to phosphinothricin and sulphonamide based herbicides (bar and sul respectively; EP-A-242246, EP-A-0249637), incorporated herein by reference; and screenable markers such as beta-glucuronidase (GB2197653), incorporated herein by reference, luciferase and green fluorescent protein (GFP). The marker gene may be controlled by a second promoter, which allows expression in cells, which may or may not be in the seed, thereby allowing the selection of cells or tissue containing the marker at any stage of development of the plant. Suitable second promoters are the promoter of nopaline synthase gene of Agrobacterium and the promoter derived from the gene which encodes the 35S cauliflower mosaic virus (CaMV) transcript. However, any other suitable second promoter may be used.

Commercially Desirable Traits

In one embodiment the plants of the present invention have reduced total alkaloid content and/or reduced content of one or more alkaloids selected from nicotine, nornicotine, anabasine, myosmine and anatabine and/or reduced nicotine, whilst the flavour characteristics and/or other commercially desirable traits are at least maintained. In one embodiment the plants of the present invention produce leaves of a similar grade and/or quality to plants which have not been modified according to the invention.

In one embodiment the plants of the present invention have reduced nicotine content without a significant change in the flavour characteristics of the plant (e.g. compared with the same plant which has not been modified in accordance with the present invention).

In one embodiment the plants of the present invention have a reduced nicotine content without a significant change (e.g. decrease) in other commercially desirable traits of the plant (e.g. compared with the same plant which has not been modified in accordance with the present invention). In particular the yield of the modified plant is preferably not reduced compared with the same plant which has not been modified in accordance with the present invention.

Therefore in one embodiment the methods and uses of the present invention relate to reducing total alkaloid content and/or reducing one or more alkaloids selected from nicotine, nornicotine, anabasine and anatabine and/or reducing nicotine, whilst maintain the flavour characteristics and/or other commercially desirable traits (e.g. yield).

The term “commercially desirable traits” will include traits such as yield, mature plant height, harvestable leaf number, average node length, cutter leaf length, cutter leaf width, quality, abiotic (for instance drought) stress tolerance, herbicide tolerance and/or biotic (for instance insect, bacteria or fungus) stress tolerance.

The term “commercially desirable traits” as taught herein will include traits such as drought resistance, pest resistance, mature plant height, harvestable leaf number, average node length, cutter leaf length, cutter leaf width, and yield which are comparable to those said traits in the flue-cured parent of a comparable plant when grown in similar field conditions.

Unless specified otherwise, used herein, tobacco yield refers to cured leaf yield which is calculated based on the weight of cured tobacco leaves per acre under standard field conditions following standard agronomic and curing practice.

In one aspect, a plant (e.g. a tobacco plant) of the present invention has a yield between 50% and 150%, between 55% and 145%, between 60% and 140%, between 65% and 135%, between 70% and 130%, between 75% and 125%, between 80% and 120%, between 85% and 115%, between 90% and 110%, between 95% and 105%, between 50% and 100%, between 55% and 100%, between 60% and 100%, between 65% and 100%, between 70% and 100%, between 75% and 100%, between 80% and 100%, between 85% and 100%, between 90% and 100%, between 95% and 100%, between 100% and 150%, between 105% and 150%, between 110% and 150%, between 115% and 150%, between 120% and 150%, between 125% and 150%, between 130% and 150%, between 135% and 150%, between 140% and 150%, or between 145% and 150% of the yield of a comparable plant when grown in similar field conditions.

In another aspect, the plant (e.g. a tobacco plant) yield of the present invention is approximately 1.5, 1.6, 1.7, 1.8, 1.9, 2.0, 2.1, 2.2, 2.3, 2.4, 2.5, 2.6, 2.7, 2.8, 2.9, or 3.0 times of the yield of a comparable plant when grown in similar field conditions.

In another aspect, the yield of a tobacco plant of the present invention is comparable to the yield of a comparable plant when grown in similar field conditions.

In another aspect, the yield of a tobacco plant of the present invention is comparable to the yield of the flue cured comparable plant when grown in similar field conditions.

In one aspect, a tobacco plant of the present invention provides a yield selected from the group consisting of about between 1200 and 3500, between 1300 and 3400, between 1400 and 3300, between 1500 and 3200, between 1600 and 3100, between 1700 and 3000, between 1800 and 2900, between 1900 and 2800, between 2000 and 2700, between 2100 and 2600, between 2200 and 2500, and between 2300 and 2400 lbs/acre.

In another aspect, a tobacco plant of the present invention provides a yield selected from the group consisting of about between 1200 and 3500, between 1300 and 3500, between 1400 and 3500, between 1500 and 3500, between 1600 and 3500, between 1700 and 3500, between 1800 and 3500, between 1900 and 3500, between 2000 and 3500, between 2100 and 3500, between 2200 and 3500, between 2300 and 3500, between 2400 and 3500, between 2500 and 3500, between 2600 and 3500, between 2700 and 3500, between 2800 and 3500, between 2900 and 3500, between 3000 and 3500, and between 3100 and 3500 lbs/acre.

In a further aspect, a tobacco plant of the present invention provides a yield selected from the group consisting of about between 1200 and 3500, between 1200 and 3400, between 1200 and 3300, between 1200 and 3200, between 1200 and 3100, between 1200 and 3000, between 1200 and 2900, between 1200 and 2800, between 1200 and 2700, between 1200 and 2600, between 1200 and 2500, between 1200 and 2400, between 1200 and 2300, between 1200 and 2200, between 1200 and 2100, between 1200 and 2000, between 1200 and 1900, between 1200 and 1800, between 1200 and 1700, between 1200 and 1600, between 1200 and 1500, and between 1200 and 1400 lbs/acre.

Tobacco Plants

The present invention provides methods, uses directed to plants (e.g. tobacco plants) as well as a cell (e.g. a tobacco cell), a plant (e.g. a tobacco plant) and a plant propagation material.

The term “tobacco” as used herein refers to a plant in the genus Nicotiana that is used in the production of delivery systems. Non-limiting examples of suitable “tobacco” plants include N. tabacum and N. rustica (for example, N. tabacum L., LA B21, LN KY171, TI 1406, Basma, Galpao, Perique, Beinhart 1000-1, and Petico).

In one embodiment a suitable tobacco plant may be any N. tabacum germplasm, line or variety.

In another embodiment a suitable tobacco plant may be a non tabacum species.

The tobacco material can be derived or obtained from varieties of Nicotiana tabacum types, commonly known as Burley varieties, flue or bright varieties and dark varieties. In some embodiments, the tobacco material is derived from a Burley, Virginia or a dark tobacco plant. The tobacco plant may be selected from Burley tobacco, rare tobacco, specialty tobacco, expanded tobacco or the like.

The use of tobacco cultivars and elite tobacco cultivars is also contemplated herein. The tobacco plant for use herein may therefore be a tobacco variety or elite tobacco cultivar. Particularly useful Nicotiana tabacum varieties include Flue-cured Virginia type, Burley type, and Oriental type.

In some embodiments, the tobacco plant may be, for example, selected from one or more of the following varieties: L. cultivar T.I. 1068, AA 37-1, B 13P, Xanthi (Mitchell-Mor), KT D #3 Hybrid 107, Bel-W3, 79-615, Samsun Holmes N N, F4 from cross BU21×Hoja Parado, line 97, KTRDC #2 Hybrid 49, KTRDC #4 Hybrid 1 10, Burley 21, PM016, KTRDC #5 KY 160 SI, KTRDC #7 FCA, KTRDC #6 TN 86 SI, PM021, K 149, K 326, K 346, K 358, K 394, K 399, K 730, KY 10, KY 14, KY 160, KY 17, KY 8959, KY 9, KY 907, MD 609, McNair 373, NC 2000, PG 01, PG 04, P01, P02, P03, RG 11, RG 17, RG 8, Speight G-28, TN 86, TN 90, VA 509, AS44, Banket A1, Basma Drama B84/31, Basma I Zichna ZP4/B, Basma Xanthi BX 2A, Batek, Besuki Jember, C104, Coker 319, Coker 347, Criollo Misionero, PM092, Delcrest, Djebel 81, DVH 405, Galpao Comum, HBO4P, Hicks Broadleaf, Kabakulak Elassona, PM102, Kutsage E1, KY 14×L8, KY 171, LA BU 21, McNair 944, NC 2326, NC 71, NC 297, NC 3, PVH 03, PVH 09, PVH 19, PVH 21 10, Red Russian, Samsun, Saplak, Simmaba, Talgar 28, PM132, Wislica, Yayaldag, NC 4, TR Madole, Prilep HC-72, Prilep P23, Prilep PB 156/1, Prilep P12-2/1, Yaka JK-48, Yaka JB 125/3, TI-1068, KDH-960, TI-1070, TW136, PM204, PM205, Basma, TKF 4028, L8, TKF 2002, TN 90, GR141, Basma xanthi, GR149, GR153, and Petit Havana.

Non-limiting examples of varieties or cultivars are: BD 64, CC 101, CC 200, CC 27, CC 301, CC 400, CC 500, CC 600, CC 700, CC 800, CC 900, Coker 176, Coker 319, Coker 371 Gold, Coker 48, CD 263, DF91 1, DT 538 LC, Galpao tobacco, GL 26H, GL 350, GL 600, GL 737, GL 939, GL 973, HB 04P, HB 04P LC, HB3307PLC, Hybrid 403LC, Hybrid 404LC, Hybrid 501 LC, K 149, K 326, K 346, K 358, K394, K 399, K 730, KDH 959, KT 200, KT204LC, KY10, KY14, KY 160, KY 17, KY 171, KY 907, KY907LC, KTY14×L8 LC, Little Crittenden, McNair 373, McNair 944, msKY 14×L8, Narrow Leaf Madole, Narrow Leaf Madole LC, NBH 98, N-126, N-777LC, N-7371 LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, PD 7302 LC, PD 7309 LC, PD 7312 LC ‘Periq'e’ tobacco, PVH03, PVH09, PVH19, PVH50, PVH51, R 610, R 630, R 7-1 1, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, VA359, AA 37-1, B 13P, Xanthi (Mitchell-Mor), Bel-W3, 79-615, Samsun Holmes NN, KTRDC number 2 Hybrid 49, Burley 21, KY 8959, KY 9, MD 609, PG 01, PG 04, P01, P02, P03, RG 1 1, RG 8, VA 509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpao Comum, HBO4P, Hicks Broadleaf, Kabakulak Elassona, Kutsage E1, LA BU 21, NC 2326, NC 297, PVH 21 10, Red Russian, Samsun, Saplak, Simmaba, Talgar 28, Wislica, Yayaldag, Prilep HC-72, Prilep P23, Prilep PB 156/1, Prilep P12-2/1, Yaka JK-48, Yaka JB 125/3, TI-1068, KDH-960, TI-1070, TW136, Basma, TKF 4028, L8, TKF 2002, GR141, Basma xanthi, GR149, GR153, Petit Havana. Low converter subvarieties of the above, even if not specifically identified herein, are also contemplated.

The plant may be a hybrid produced by crossing any varieties disclosed herein.

The tobacco plant may be a Burley, Flue-cured Virginia, or Oriental.

In one embodiment the plant propagation material may be obtainable from a plant (e.g. a tobacco plant) of the invention. A “plant propagation material” as used herein refers to any plant matter taken from a plant from which further plants may be produced. Suitably the plant propagation material may be a seed. Suitably the plant propagation material may be pollen.

In one embodiment the cell (e.g. a tobacco cell), plant (e.g. a tobacco plant) or part thereof and/or plant propagation material of the invention may comprise modulated activity or expression of a Nic3 gene (or a Nic3 gene and a Nic1 ERF gene and/or a Nic2 ERF gene). In another embodiment the cell (e.g. tobacco cell), plant (e.g. tobacco plant) and/or plant propagation material may comprise a construct or vector according to the invention. In another embodiment the cell (e.g. tobacco cell), plant (e.g. tobacco plant) and/or plant propagation material may be obtainable (e.g. obtained) by a method according to the invention.

Suitably a plant (e.g. a tobacco plant) or part thereof according to the present invention may comprise modulated activity or expression of a Nic3 ERF gene (or a Nic3 gene and a Nic1 ERF gene and/or a Nic2 ERF gene), when compared to a plant (e.g. a tobacco plant) or part thereof that has not been modified to modulate the activity or expression of a Nic3 gene (or a Nic3 gene and a Nic1 ERF gene and/or a Nic2 ERF gene).

In one embodiment the plant (e.g. tobacco plant) or part thereof in accordance with the present invention comprises a cell (e.g. a tobacco cell) of the invention. In another embodiment the plant propagation material may be obtainable (e.g. obtained) from a plant (e.g. a tobacco plant) of the invention.

In one embodiment there is provided the use of a cell (e.g. a tobacco cell) as provided for in the foregoing embodiments for production of a product (e.g. a delivery system). Additionally, there is provided the use of a plant (e.g. a tobacco plant) as described herein to breed a plant (e.g. a tobacco plant).

The present invention also provides in another embodiment the use of a plant (e.g. a tobacco plant) of the foregoing embodiments for the production of a product (e.g. a delivery system). In another embodiment there is provided the use of a plant (e.g. a tobacco plant) of the invention to grow a crop. In one embodiment the use of a Nic3 gene (or a Nic3 gene and a Nic1 ERF gene and/or a Nic2 ERF gene) according to the present invention results in modulation of the alkaloid content of a plant (e.g. a tobacco plant).

In one embodiment the method or use of a Nic3 gene (or a Nic3 gene and a Nic1 ERF gene and/or a Nic2 ERF gene) according to the present invention may result in the modulation of the alkaloid content. In another embodiment the use of a Nic3 gene (or a Nic3 gene and a Nic1 ERF gene and/or a Nic2 ERF gene) (e.g. decreased activity or expression thereof) may result in a decrease in content of one or more alkaloids. Suitably the content of one or more of anatabine, anabasine, myosmine, nornicotine or nicotine may be reduced. Suitably the nicotine content is reduced. Suitably this may be observed when Nic3 gene activity or expression (or activity of a Nic3 gene and a Nic1 ERF gene and/or a Nic2 ERF gene), is decreased compared to wild type plants.

In another embodiment the method or use of a Nic3 gene (or a Nic3 gene and a Nic1 ERF gene and/or a Nic2 ERF gene) (e.g. increased activity or expression thereof) may result in an increase in content of one or more alkaloids. Suitably the content of one or more of anatabine, anabasine, nornicotine or nicotine may be increased. Suitably the nicotine content is reduced.

In one embodiment the plant (e.g. tobacco plant) or part thereof, e.g. the leaf, or harvested leaf or harvested processed leaf, or cells or products (e.g. delivery systems) comprise a modified (e.g. mutated or deleted) Nic3 gene of the present invention (or a modified (e.g. mutated or deleted) Nic3 gene in combination with a modified (e.g. mutated or deleted) Nic1 ERF gene and/or a modified (e.g. mutated or deleted) Nic2 ERF gene in accordance with the present invention).

In one embodiment the present invention provides a tobacco cell culture (e.g. in in vitro culture).

The tobacco cell culture may be a tobacco cell suspension culture. These tobacco cells cultured in vitro may be incorporated into a delivery system, e.g. as a substitute for conventional tobacco particles, shreds, fine cut or long cut tobacco lamina, as an additive ingredient or as both a substitute and an additive.

In one embodiment there is provided the use of a tobacco cell culture, e.g. a harvested and/or processed tobacco cell culture, or an extract therefrom according to the present invention for the production of a delivery system.

The tobacco cells harvested from an in vitro culture may be dried, e.g. freeze-dried, for example to produce a powder.

The skilled person will be aware of known methods for establishing in vitro cultures of tobacco cells. By way of example only the following method may be used: collecting seeds from a tobacco plant of interest and sterilising their exterior to eliminate unwanted organisms, planting said seeds to grown a tobacco plant of interest, removing tissue from the tobacco plant (for example, from the tobacco stem) for use as an explant, establishing a callus culture form the tobacco explant, establishing a cell suspension culture from the callus culture, and harvesting culture material (e.g. including tobacco cells) to produce a tobacco cell culture.

The tobacco cells can be harvested by various methods, including filtration, e.g. vacuum filtration. The sample may be washed in the filter by adding water and the remaining liquid removed with the filtration, e.g. vacuum filtration.

The harvested tobacco cell culture may be further processed, e.g. dried, such as air-dried and/or freeze-dried. The harvested tobacco cell culture or dried harvested tobacco cell culture or an extract therefrom may be incorporated into delivery systems according to the present invention.

In one embodiment, the present invention provides a tobacco plant or part thereof for use in molecular farming. Suitably, a plant or part thereof modified in accordance with the present invention may be used in the manufacture of proteins such as therapeutics e.g. antibiotics, virus like particles, neutraceuticals or small molecules.

In one embodiment, the present invention provides a method for the production of proteins (e.g. therapeutic proteins); the method comprising modifying a plant or part thereof capable of producing said protein (e.g. therapeutic protein) by modulating the activity or expression of at least one Nic3 ERF gene; or at least one Nic3 gene and at least one Nic1 ERF gene and/or at least one Nic2 ERF gene as described herein and culturing the plant under conditions sufficient to allow the production of said protein (e.g. therapeutic protein).

In one aspect, the present invention provides a method of introgressing a low nicotine trait into a tobacco variety, the method comprising,

a) crossing a first tobacco variety comprising g a low nicotine trait with a second tobacco variety without the low nicotine trait to produce one or more progeny tobacco plants;

b) genotyping the one or more progeny tobacco plants for a polymorphic marker linked to the low nicotine trait, there the polymorphic marker is within 20 cM, 10 cM, 5 cM, 4 cM, 3 cM, 2 cM, 1 cM 0.5 cM or less than 0.5 cM of a Nic3 locus; and

c) selecting a progeny tobacco plant comprising the low nicotine trait.

In an aspect this method optionally comprises selecting simultaneously or concurrently for one of more molecular markers associated with or closely linked to a Nic1 locus and/or one or more molecular markers associated with or closely linked to a Nic2 locus.

Products

The present invention also provides for products obtainable or obtained from tobacco according to the present invention. Products are provided which are obtainable or obtained from a tobacco plant in which Nic3 gene activity or expression (or activity or expression of a Nic3 gene and Nic1 ERF gene and/or a Nic2 ERF gene) has been modulated and which comprises modulated alkaloid content (e.g. reduced nicotine content).

In one aspect, the present invention comprises a delivery system comprising a tobacco plant or part thereof or plant cell according to the invention;

a tobacco plant or part thereof propagated from a tobacco plant propagation material according to the invention; a harvested leaf of a plant according to the invention; a processed leaf according to the invention; or a plant produced by the method according to the invention.

As used herein, the term “delivery system” is intended to encompass systems that deliver at least one substance to a user, and includes:

combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokable material);

non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and

aerosol-free delivery systems that deliver the at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

According to the present disclosure, a “combustible” aerosol provision system is one where a constituent aerosol-generating material of the aerosol provision system (or component thereof) is combusted or burned during use in order to facilitate delivery of at least one substance to a user.

In some embodiments, the delivery system is a combustible aerosol provision system, such as a system selected from the group consisting of a cigarette, a cigarillo and a cigar.

In some embodiments, the disclosure relates to a component for use in a combustible aerosol provision system, such as a filter, a filter rod, a filter segment, a tobacco rod, a spill, an aerosol-modifying agent release component such as a capsule, a thread, or a bead, or a paper such as a plug wrap, a tipping paper or a cigarette paper.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller.

The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

Suitably, the delivery system may be prepared from (e.g. may comprise) a tobacco plant or a part thereof according to the present invention.

Suitably, the delivery system may be prepared from a tobacco cell culture according to the present invention.

Suitably, the delivery system may be prepared from (e.g. may comprise) a tobacco plant or part thereof propagated from a tobacco plant propagation material according to the present invention.

Suitably, the delivery system may be prepared from (e.g. may comprise) a harvested leaf of a tobacco plant according to the present invention.

Suitably, the delivery system may be prepared from (e.g. may comprise) a processed tobacco leaf according to the present invention.

Suitably, the delivery system may be prepared from (e.g. may comprise) a cured tobacco material according to the present invention.

Suitably, the delivery system may be prepared from (e.g. may comprise) a tobacco blend according to the present invention.

In one embodiment, the delivery system is a combustible smoking article, selected from the group consisting of a cigarette, a cigarillo and a cigar.

In one embodiment, the delivery system comprises one or more components of a combustible smoking article, such as a filter, a filter rod, a filter rod segments, tobacco, a tobacco rod, a tobacco rod segment, a spill, an additive release component such as a capsule, a thread, beads, a paper such as a plug wrap, a tipping paper or a cigarette paper.

In one embodiment, the delivery system is a non-combustible aerosol provision system.

In one embodiment, the delivery system comprises one or more components of a non-combustible aerosol provision system, such as a heater and an aerosolizable substrate.

In one embodiment, the aerosol provision system is an electronic cigarette also known as a vaping device.

In one embodiment the electronic cigarette comprises a heater, a power supply capable of supplying power to the heater, an aerosolizable substrate such as a liquid or gel, a housing and optionally a mouthpiece.

In one embodiment the aerosolizable substrate is contained in a substrate container. In one embodiment the substrate container is combined with or comprises the heater.

In one embodiment, the delivery system is a heating product which releases one or more compounds by heating, but not burning, a substrate material. The substrate material is an aerosolizable material which may be for example tobacco or other non-delivery systems, which may or may not contain nicotine. In one embodiment, the heating product is a tobacco heating product.

In one embodiment, the heating product is an electronic device.

In one embodiment, the tobacco heating product comprises a heater, a power supply capable of supplying power to the heater, an aerosolizable substrate such as a solid or gel material.

In one embodiment the heating product is a non-electronic article.

In one embodiment the heating product comprises an aerosolizable substrate such as a solid or gel material and a heat source which is capable of supplying heat energy to the aerosolizable substrate without any electronic means, such as by burning a combustion material, such as charcoal.

In one embodiment the heating product also comprises a filter capable of filtering the aerosol generated by heating the aerosolizable substrate.

In some embodiments the aerosolizable substrate material may comprise a vapour or aerosol generating agent or a humectant, such as glycerol, propylene glycol, triacetin or diethylene glycol.

In one embodiment, the delivery system is a hybrid system to generate aerosol by heating, but not burning, a combination of substrate materials. The substrate materials may comprise for example solid, liquid or gel which may or may not contain nicotine. In one embodiment, the hybrid system comprises a liquid or gel substrate and a solid substrate. The solid substrate may be for example tobacco or other non-delivery systems, which may or may not contain nicotine. In one embodiment, the hybrid system comprises a liquid or gel substrate and tobacco.

In another embodiment, the product may comprise a construct of the invention which modulates activity or expression of at least one Nic3 gene and thereby decreases alkaloid content (e.g. nicotine content) when expressed in a plant (e.g. tobacco plant).

In another embodiment, the product may comprise one or more constructs of the invention which modulates Nic3 gene activity or expression (or Nic3 gene activity or expression and Nic1 ERF gene activity or expression and/or Nic2 ERF gene activity or expression) wherein said product has modulated alkaloid content (e.g. reduced nicotine content).

In one embodiment there is provided the use of a plant of the invention (e.g. a tobacco plant) to produce leaf (e.g. tobacco leaf). Suitably the leaf (e.g. tobacco leaf) may be subjected to downstream applications such as processing. Thus in one embodiment the use of the foregoing embodiment may provide a processed leaf (e.g. a processed tobacco leaf). Suitably the tobacco leaf may be subjected to curing, fermenting, pasteurising or combinations thereof.

In another embodiment the leaf (e.g. tobacco leaf) may be cut. In some embodiments the leaf (e.g. tobacco leaf) may be cut before or after being subjected to curing, fermenting, pasteurising or combinations thereof.

In one embodiment the present invention provides a harvested leaf of a plant of the invention (e.g. a tobacco plant). In one embodiment the harvested leaf may be obtainable from a plant (e.g. a tobacco plant) which has modulated Nic3 gene activity or expression (or modulated Nic3 and Nic1 ERF and/or Nic2 ERF gene activity or expression). Suitably the harvested leaf has modulated alkaloid content. In a further embodiment the harvested leaf may be obtainable (e.g. obtained) from a plant (e.g. a tobacco plant) propagated from a propagation material of the present invention. In another embodiment there is provided a harvested leaf obtainable from a method or use of the present invention. Suitably the harvested leaf may be a cut harvested leaf. In some embodiments the harvested leaf may comprise viable cells (e.g. viable tobacco cells). In some embodiments the harvest leaf does not comprise viable cells (e.g. viable tobacco cells). In other embodiments the harvested leaf may be subjected to further processing.

Some tobacco plants may be harvested by cutting the stalks and harvesting all of the leaves simultaneously (e.g. as with burley tobacco). Other tobacco plants (e.g. flue cured tobacco) may be harvested in stages in a process such as priming, wherein individual leaves are removed from the stalk as they ripen.

As used herein “priming” refers to the removal of leaves from tobacco plants. This may refer to the removal of mature or ripe leaves of flue cured plants.

There is also provided a processed leaf (e.g. a processed tobacco leaf). The processed leaf (e.g. processed tobacco leaf) may be obtainable from a plant of the invention (e.g. tobacco plant).

Suitably the processed leaf may be obtainable from a plant obtained in accordance with any of the methods and/or uses of the present invention. In one embodiment the processed leaf (e.g. processed tobacco leaf) may be obtainable from a plant (e.g. tobacco plant) which has modulated Nic3 gene activity or expression (or Nic3 and Nic1 ERF gene and/or Nic2 ERF gene activity or expression) and modulated alkaloid content, preferably when compared to a control leaf i.e. compared to a leaf from a plant (e.g. tobacco plant) which has not been modified according to the invention. The processed leaf (e.g. processed tobacco leaf) may comprise a modulation in Nic3 gene activity or expression (or Nic3 and Nic1 ERF gene and/or a Nic2 ERF gene activity or expression) and modulated alkaloid content.

In another embodiment the processed leaf (e.g. processed tobacco leaf) may be obtainable from a plant (e.g. tobacco plant) propagated from a plant (e.g. tobacco plant) propagation material according to the present invention. The processed leaf (e.g. processed tobacco leaf) of the present invention is obtainable by processing a harvested leaf of the invention.

The term “processed leaf” as used herein refers to a leaf that has undergone one or more processing steps to which leaves are subjected to in the art. A “processed leaf” comprises no or substantially no viable cells.

The term “processed tobacco leaf” as used herein refers to a tobacco leaf that has undergone one or more processing steps to which tobacco is subjected to in the art. A “processed tobacco leaf” comprises no or substantially no viable cells.

The term “viable cells” refers to cells which are able to grow and/or are metabolically active. Thus, if a cell is said to not be viable, also referred to as “non-viable” then a cell does not display the characteristics of a viable cell.

The term “substantially no viable cells” means that less than about 5% of the total cells are viable. Preferably, less than about 3%, more preferably less than about 1%, even more preferably less than about 0.1% of the total cells are viable.

In one embodiment the processed tobacco leaf may be processed by one or more of: curing, fermenting and/or pasteurising. Suitably the processed tobacco leaf may be processed by curing.

Tobacco leaf may be cured by any method known in the art. In one embodiment tobacco leaf may be cured by one or more of the curing methods selected from the group consisting of: air curing, fire curing, flue curing and sun curing. Suitably the tobacco leaf may be air cured. Suitably the tobacco leaf may be flue cured.

Typically air curing is achieved by hanging tobacco leaf in well-ventilated barns and allowing to dry. This is usually carried out over a period of four to eight weeks. Air curing is especially suitable for Burley tobacco.

Suitably the tobacco leaf may be fire cured. Fire curing is typically achieved by hanging tobacco leaf in large barns where fires of hardwoods are kept on continuous or intermittent low smoulder and usually takes between three days and ten weeks, depending on the process and the tobacco.

In another embodiment the tobacco leaf may be flue cured. Flue curing may comprise stringing tobacco leaves onto tobacco sticks and hanging them from tier-poles in curing barns. The barns usually have a flue which runs from externally fed fire boxes. Typically this results in tobacco that has been heat-cured without being exposed to smoke. Usually the temperature will be raised slowly over the course of the curing with the whole process taking approximately 1 week.

Suitably the tobacco leaf may be sun cured. This method typically involves exposure of uncovered tobacco to the sun.

Suitably the processed tobacco leaf may be processed by fermenting. Fermentation can be carried out in any manner known in the art. Typically during fermentation, the tobacco leaves are piled into stacks (a bulk) of cured tobacco covered in e.g. burlap to retain moisture. The combination of the remaining water inside the leaf and the weight of the tobacco generates a natural heat which ripens the tobacco. The temperature in the centre of the bulk is monitored daily. In some methods every week, the entire bulk is opened. The leaves are then removed to be shaken and moistened and the bulk is rotated so that the inside leaves go outside and the bottom leaves are placed on the top of the bulk. This ensures even fermentation throughout the bulk. The additional moisture on the leaves, plus the actual rotation of the leaves themselves, generates heat, releasing the tobacco's natural ammonia and reducing nicotine, while also deepening the colour and improving the tobacco's aroma. Typically the fermentation process continues for up to 6 months, depending on the variety of tobacco, stalk position on the leaf, thickness and intended use of leaf.

Suitably the processed tobacco leaf may be processed by pasteurising. Pasteurising may be particularly preferred when the tobacco leaf will be used to make a smokeless delivery system, most preferably snus. Tobacco leaf pasteurisation may be carried out by any method known in the art. For example, pasteurisation may be carried out as detailed in J Foulds, L Ramstrom, M Burke, K Fagerstrom. Effect of smokeless tobacco (snus) on smoking and public health in Sweden. Tobacco Control (2003) 12: 349-359, the teaching of which is incorporated herein by reference.

During the production of snus, pasteurisation is typically carried out by a process in which the tobacco is heat treated with steam for 24-36 hours (reaching temperatures of approximately 100° C.). This results in an almost sterile product and without wishing to be bound by theory one of the consequences of this is believed to be a limitation of further TSNA formation.

In one embodiment the pasteurisation may be steam pasteurisation.

In some embodiments the processed tobacco leaf may be cut. The processed tobacco leaf may be cut before or after processing. Suitably, the processed tobacco leaf may be cut after processing.

In some embodiments the tobacco plant, harvested leaf of a tobacco plant and/or processed tobacco leaf may be used to extract nicotine. The extraction of nicotine can be achieved using any method known in the art. For example, a method for extracting nicotine from tobacco is taught in U.S. Pat. No. 2,162,738 which is incorporated herein by reference.

In one aspect, the present invention provides cured tobacco material made from a tobacco plant or part thereof according to the invention.

In another aspect, the present invention provides a tobacco blend comprising tobacco material made from a tobacco plant or part thereof according to the present invention. In one aspect, the present invention provides a tobacco blend comprising cured tobacco material according to the present invention.

Suitably, the tobacco blend according to the present invention may comprise approximately 10%, 20%, 30%, 40%, 50%, 60%, 70%, 80% or 90% tobacco from a tobacco plant or part thereof according to the present invention. Suitably, the tobacco blend may comprise approximately 10% tobacco from a tobacco plant or part thereof according to the present invention. Suitably, the tobacco blend may comprise approximately 20% tobacco from a tobacco plant or part thereof according to the present invention. Suitably, the tobacco blend may comprise approximately 30% tobacco from a tobacco plant or part thereof according to the present invention. Suitably, the tobacco blend may comprise approximately 40% tobacco from a tobacco plant or part thereof according to the present invention. Suitably, the tobacco blend may comprise approximately 50% tobacco from a tobacco plant or part thereof according to the present invention. Suitably, the tobacco blend may comprise approximately 60% tobacco from a tobacco plant or part thereof according to the present invention. Suitably, the tobacco blend may comprise approximately 70% tobacco from a tobacco plant or part thereof according to the present invention. Suitably, the tobacco blend may comprise approximately 80% tobacco from a tobacco plant or part thereof according to the present invention. Suitably, the tobacco blend may comprise approximately 90% tobacco from a tobacco plant or part thereof according to the present invention.

In one aspect, a tobacco blend product of the present invention comprises at least about 5, 10, 20, 30, 40, 50, 60, 70, 80, 90, or 95 percent by dry weight of tobacco cured from a tobacco plant or part thereof according to the present invention.

Suitably the cured tobacco material may be air cured. Suitably the cured tobacco material may be flue cured. Suitably the cured tobacco material may be sun cured.

A delivery system or smoking article according to the present invention may comprise the tobacco material (e.g. cured tobacco material) according to the present invention.

In another aspect the present invention provides a delivery system. Suitably, a delivery system may be a blended delivery system. In one embodiment the delivery system may be prepared from a tobacco plant of the invention or a part thereof. In one embodiment the delivery system may be prepared from a tobacco plant which has modulated Nic3 gene activity or expression or Nic3 gene and Nic1 ERF gene and/or Nic2 ERF gene activity or expression. The delivery system may comprise a reduction in Nic1 ERF gene activity or expression and reduced alkaloid content. Suitably the tobacco plant or part thereof may be propagated from a tobacco plant propagation material according to the present invention.

The term “part thereof” as used herein in the context of a plant (e.g. a tobacco plant) refers to a portion of the plant (e.g. tobacco plant). Preferably the “part thereof” is a leaf of a plant (e.g. of a tobacco plant).

In another embodiment the delivery system may be prepared from a harvested leaf of the invention. In a further embodiment the delivery system may be prepared from a processed tobacco leaf of the invention. Suitably the delivery system may be prepared from a tobacco leaf processed by one or more of: curing, fermenting and/or pasteurising. Suitably the delivery system may comprise a cut tobacco leaf, optionally processed as per the foregoing embodiment.

In one embodiment the delivery system may be a smoking article. As used herein, the term “smoking article” can include smokeable products, such as rolling tobacco, cigarettes, cigars and cigarillos whether based on tobacco, tobacco derivatives, expanded tobacco, reconstituted tobacco or tobacco substitutes.

In another embodiment the delivery system may be a smokeless delivery system. The term “smokeless delivery system” as used herein refers to a delivery system that is not intended to be smoked and/or subjected to combustion. In one embodiment a smokeless delivery system may include snus, snuff, chewing tobacco or the like.

In a further embodiment the delivery system may be a tobacco heating device or hybrid device or e-cigarettes or the like. Typically in heating devices or hybrid devices, an aerosol is generated by the transfer of heat from a heat source to a physically separate aerosol-forming substrate or material, which may be located within, around or downstream of the heat source. During smoking, volatile compounds are released from the aerosol-forming substrate by heat transfer from the heat source and entrained in air drawn through the smoking article. As the released compounds cool, they condense to form an aerosol that is inhaled by the user.

Aerosol-generating articles and devices for consuming or smoking tobacco heating devices are known in the art. They can include, for example, electrically heated aerosol-generating devices in which an aerosol is generated by the transfer of heat from one or more electrical heating elements of the aerosol-generating device to the aerosol-forming substrate of a tobacco heating device. Suitably the tobacco heating device may be an aerosol-generating device.

Preferably the tobacco heating device may be a heat-not-burn device. Heat-not-burn devices are known in the art and release compounds by heating, but not burning, tobacco. An example of a suitable, heat-not-burn device may be one taught in WO2013/034459 or GB2515502 which are incorporated herein by reference.

In one embodiment the aerosol-forming substrate of a tobacco heating device may be a delivery system in accordance with the present invention.

In one embodiment the tobacco heating device may be a hybrid device.

Molecular Farming

The present invention may be particularly useful in the field of plant molecular farming, where plants, or parts thereof or plant cells (such as tobacco and other Nicotiana spp.) are used for the production of proteins, peptides, and metabolites e.g. for the production of therapeutics and pharmaceuticals such as antibiotics, virus like particles, or neutraceuticals or small molecules.

As used herein “molecular farming” relates to the production of recombinant proteins and/or other secondary metabolites in plants, or parts thereof or plant cells.

Suitably, molecular farming (or biopharming) may comprise modifying a plant or part thereof or plant cell by introducing a nucleic acid sequence which encodes a recombinant protein and cultivating said plant or part thereof or plant cell comprising the nucleic acid sequence under conditions which allow the expression of said recombinant protein. Suitably, the method may further comprise and extraction and optionally, purification of the recombinant protein from the plant or part thereof or plant cell. Suitably, molecular farming (or biopharming) may comprise modifying a plant or part thereof or plant cell by introducing a nucleic acid sequence which encodes a recombinant protein, cultivating said plant or part thereof or plant cell comprising the nucleic acid sequence under conditions which allow the expression of said recombinant protein, and extraction and purification of the recombinant protein from the plant or part thereof or plant cell.

Suitably, molecular farming (or biopharming) may comprise cultivating a plant or part thereof or plant cell under conditions which allow the expression of a secondary metabolite. Suitably, the method may further comprise and extraction and optionally, purification of the secondary metabolite from the plant or part thereof or plant cell. Suitably, molecular farming (or biopharming) may comprise cultivating a plant or part thereof or plant cell under conditions which allow the expression of a secondary metabolite, and extraction and purification of the recombinant protein from the plant or part thereof or plant cell.

Methods for extracting and purifying recombinant proteins and/or secondary metabolites from plants or parts thereof or plant cells are known in the art, for example: in U.S. Pat. Nos. 9,220,295; 9,289,011; 9,175,052 and US Patent Application Number: 2016/0029663.

Thus a plant or part thereof or plant cell according to the present invention may be used for molecular farming. A plant or part thereof or plant cell according to the present invention may be used to reduce or eliminate the presence of nicotine and/or other nicotinic alkaloids in the plant or part thereof or plant cell. A plant or part thereof or plant cell according to the present invention may be used to reduce or eliminate the presence of nicotine and/or other nicotinic alkaloids in the product extracted and/or purified from the plant or part thereof or plant cell.

Advantageously, the use of a low nicotine plant or rootsock in molecular farming will reduce downstream processing costs associated with purification of the product from the plant or part thereof or plant cell. Tobacco plants are attractive bioreactors for the production of recombinant proteins due to their potential for large-scale and low-cost production.

Plants or parts thereof or plant cells which are suitable for use in molecular farming include but are not limited to Nicotiana spp. Suitably, the plant or plant thereof or plant cell for use in molecular farming may be Nicotiana benthamiana Suitably, the plant or plant thereof or plant cell for use in molecular farming may be Nicotiana tabacum.

In one aspect, there is provided a tobacco plant for use in molecular farming. For example the tobacco plants according to the present invention may be used for the production of recombinant proteins. Recombinant proteins which may be produced in tobacco plants include for example: antigens for the production of vaccines, antibodies, enzymes, vaccines, growth factors.

Monoclonal antibodies and fragments thereof may be produced by molecular farming using plants or parts thereof or plant cells according to the present invention, including for example immunoglobulin G (IgG) and immunoglobulin A (IgA), IgA and IgG shimmer molecules, IgG and IgA secreted molecules, single-chain variable fragment, fragment antigen-binding, and variable of heavy and light chains.

Pharmaceutical proteins may be produced by molecular farming using plants or parts thereof or plant cells according to the present invention, for example, pharmaceutical proteins which have been expressed in plants include erythropoietin, interferon, hirudin, aprotinin, Leu-enkephalin, somatotropin of human growth hormone.

Non-pharmaceutical proteins may be produced by molecular faming using plants or parts thereof or plant cells according to the present invention, for example non-pharmaceutical proteins derived from plants include avidin, trypsin, aprotinin, β-glucocerebrosidase, peroxidase and cellulose.

Suitably, the plant or part thereof or plant cell for use in molecular farming according to the present invention may comprise an average alkaloid level or average nicotine level of about 0.01%, 0.02%, 0.05%, 0.0.75%. 0.1%, 0.2%, 0.3%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3%, 4% or 5% on a dry weight basis. Suitably, the plant or part thereof or plant cell for use in molecular farming according to the present invention may comprise an average alkaloid level or average nicotine level of less than 5%, less, than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.075%, less than 0.05%, less than 0.02% or less than 0.01%.

Suitably, molecular farming according to the present invention may produce a product, extract or purified product (e.g. recombinant protein) which comprises an average alkaloid level or average nicotine level of about 0.01%, 0.02%, 0.05%, 0.0.75%. 0.1%, 0.2%, 0.3%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3%, 4% or 5% on a dry weight basis. Suitably, molecular farming according to the present invention may produce a product, extract or purified product (e.g. recombinant protein) which comprises an average alkaloid level or average nicotine level of less than 5%, less, than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.075%, less than 0.05%, less than 0.02% or less than 0.01%.

Polynucleotides/Polypeptides/Constructs

In certain embodiments of the present invention, constructs which modulate activity or expression of at least one Nic3 gene (or at least one Nic3 gene and at least one Nic1 ERF gene and/or at least one Nic2 ERF gene) may be transformed into plant cells suitably under the direction of a promoter.

In certain embodiments of the present invention, constructs which decrease (i.e. inhibit) activity or expression of a Nic3 gene (or a Nic3 gene and a Nic1 ERF gene and/or a Nic2 ERF gene) may be transformed into plant cells under the direction of a promoter. The genetic construct may be a gene editing construct or may comprise an RNAi molecule, which may comprise a small interfering RNA (siRNA) molecule, or a short hairpin loop (shRNA) molecule.

In certain embodiments of the present invention, constructs which increase activity or expression of a Nic3 gene (or a Nic3 gene and a Nic1 ERF gene and/or a Nic2 ERF gene) may be transformed into plant cells under the direction of a promoter e.g. constructs which encodes the equivalent endogenous genes.

Constructs may be introduced into plants according to the present invention by means of suitable vector, e.g. plant transformation vectors. A plant transformation vector may comprise an expression cassette comprising 5′-3′ in the direction of transcription, a promoter sequence, a construct sequence targeting a Nic3 gene (or targeting a Nic3 gene and a Nic1 ERF gene and/or a Nic2 ERF gene) and, optionally a 3′ untranslated, terminator sequence including a stop signal for RNA polymerase and a polyadenylation signal for polyadenylase. The promoter sequence may be present in one or more copies, and such copies may be identical or variants of a promoter sequence as described above. The terminator sequence may be obtained from plant, bacterial or viral genes. Suitable terminator sequences are the pea rbcS E9 terminator sequence, the nos terminator sequence derived from the nopaline synthase gene of Agrobacterium tumefaciens and the 35S terminator sequence from cauliflower mosaic virus, for example. A person skilled in the art will be readily aware of other suitable terminator sequences.

The construct of the present invention may also comprise a gene expression enhancing mechanism to increase the strength of the promoter. An example of such an enhancer element is one derived from a portion of the promoter of the pea plastocyanin gene, and which is the subject of International Patent Application No. WO 97/20056, which is incorporated herein by reference. Suitable enhancer elements may be the nos enhancer element derived from the nopaline synthase gene of Agrobacterium tumefaciens and the 35S enhancer element from cauliflower mosaic virus, for example.

These regulatory regions may be derived from the same gene as the promoter DNA sequence or may be derived from different genes, from Nicotiana tabacum or other organisms, for example from a plant of the family Solanaceae, or from the subfamily Cestroideae. All of the regulatory regions should be capable of operating in cells of the tissue to be transformed.

The promoter DNA sequence may be derived from the same gene as the gene of interest, e.g. the gene the promoter is going to direct, for instance a gene encoding a Nic3 gene of the invention, a coding sequence used in the present invention or may be derived from a different gene, from Nicotiana tabacum, or another organism, for example from a plant of the family Solanaceae, or from the subfamily Cestroideae.

The expression cassette may be incorporated into a basic plant transformation vector, such as pBIN 19 Plus, pBI 101, pKYLX71:3552, pCAMBIA2300 or other suitable plant transformation vectors known in the art. In addition to the expression cassette, the plant transformation vector will contain such sequences as are necessary for the transformation process. These may include the Agrobacterium vir genes, one or more T-DNA border sequences, and a selectable marker or other means of identifying transgenic plant cells.

The term “plant transformation vector” means a construct capable of in vivo or in vitro expression. Preferably, the expression vector is incorporated in the genome of the organism. The term “incorporated” preferably covers stable incorporation into the genome.

Techniques for transforming plants are well known within the art and include Agrobacterium-mediated transformation, for example. The basic principle in the construction of genetically modified plants is to insert genetic information in the plant genome so as to obtain a stable maintenance of the inserted genetic material. A review of the general techniques may be found in articles by Potrykus (Annu Rev Plant Physiol Plant Mol Biol [1991] 42:205-225) and Christon (AgroFood-Industry Hi-Tech March/April1994 17-27), which are incorporated herein by reference. Typically, in Agrobacterium-mediated transformation a binary vector carrying a foreign DNA of interest, i.e. a Nic3 construct, is transferred from an appropriate Agrobacterium strain to a target plant by the co-cultivation of the Agrobacterium with explants from the target plant. Transformed plant tissue is then regenerated on selection media, which selection media comprises a selectable marker and plant growth hormones. An alternative is the floral dip method (Clough & Bent, 1998 Plant J. 1998 December; 16(6):735-43, which is incorporated herein by reference) whereby floral buds of an intact plant are brought into contact with a suspension of the Agrobacterium strain containing the chimeric gene, and following seed set, transformed individuals are germinated and identified by growth on selective media. Direct infection of plant tissues by Agrobacterium is a simple technique which has been widely employed and which is described in Butcher D. N. et al., (1980), Tissue Culture Methods for Plant Pathologists, eds.: D. S. Ingrams and J. P. Helgeson, 203-208 which is incorporated herein by reference.

Further suitable transformation methods include direct gene transfer into protoplasts using polyethylene glycol or electroporation techniques, particle bombardment, micro-injection and the use of silicon carbide fibres for example. Transforming plants using ballistic transformation, including the silicon carbide whisker technique are taught in Frame B R, Drayton P R, Bagnaall S V, Lewnau C J, Bullock W P, Wilson H M, Dunwell J M, Thompson J A & Wang K (1994) which is incorporated herein by reference. Production of fertile transgenic maize plants by silicon carbide whisker-mediated transformation is taught in The Plant Journal 6: 941-948, which is incorporated herein by reference) and viral transformation techniques is taught in for example Meyer P, Heidmann I & Niedenhof I (1992), which is incorporated herein by reference. The use of cassava mosaic virus as a vector system for plants is taught in Gene 110: 213-217, which is incorporated herein by reference. Further teachings on plant transformation may be found in EP-A-0449375, incorporated herein by reference.

In a further aspect, the present invention relates to a vector system which carries a construct and introducing it into the genome of an organism, such as a plant, suitably a tobacco plant. The vector system may comprise one vector, but it may comprise two vectors. In the case of two vectors, the vector system is normally referred to as a binary vector system. Binary vector systems are described in further detail in Gynheung Anetal, (1980), Binary Vectors, Plant Molecular Biology Manual A3, 1-19, which is incorporated herein by reference.

One extensively employed system for transformation of plant cells uses the Ti plasmid from Agrobacterium tumefaciens or a Ri plasmid from Agrobacterium rhizogenes described by An et al., (1986), Plant Physiol. 81, 301-305 and Butcher D. N. et al., (1980), Tissue Culture Methods for Plant Pathologists, eds.: D. S. Ingrams and J. P. Helgeson, 203-208 which are incorporated herein by reference. After each introduction method of the desired exogenous gene according to the present invention in the plants, the presence and/or insertion of further DNA sequences may be necessary. The use of T-DNA for the transformation of plant cells has been intensively studied and is described in EP-A-120516; Hoekema, in: The Binary Plant Vector System Offset-drukkerij Kanters B. B., Amsterdam, 1985, Chapter V; Fraley, et al., Crit. Rev. Plant Sci., 4:1-46; and Anetal., EMBO J (1985) 4:277-284, incorporated herein by reference.

Plant cells transformed with construct(s) which modulate the activity or expression of a Nic3 gene (or a Nic3 gene and a Nic1 ERF gene and/or a Nic2 ERF gene) may be grown and maintained in accordance with well-known tissue culturing methods such as by culturing the cells in a suitable culture medium supplied with the necessary growth factors such as amino acids, plant hormones, vitamins, etc.

The term “transgenic plant” in relation to the present invention includes any plant that comprises a construct which modulates the activity or expression of a Nic3 gene (or of a Nic3 gene and a Nic1 ERF gene and/or a Nic2 ERF gene in combination) according to the invention. Accordingly a transgenic plant is a plant which has been transformed with a construct according to the invention. Preferably the transgenic plant exhibits modulated Nic3 gene activity or expression (or of a Nic3 gene and a Nic1 ERF gene and/or a Nic2 ERF gene activity or expression) and modulated alkaloid content, according to the present invention. The term “transgenic plant” does not cover native nucleotide coding sequences in their natural environment when they are under the control of their native promoter which is also in its natural environment.

In one aspect, a Nic3 gene, a Nic1 ERF gene, a Nic2 ERF gene, a construct, plant transformation vector or plant cell according to the present invention is in an isolated form. The term “isolated” means that the sequence is at least substantially free from at least one other component with which the sequence is naturally associated in nature and as found in nature.

In one aspect, a Nic3 gene, a Nic1 ERF gene, a Nic2 ERF gene, a construct, plant transformation vector or plant cell according to the invention is in a purified form. The term “purified” means in a relatively pure state, e.g. at least about 90% pure, or at least about 95% pure or at least about 98% pure.

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention, i.e. the Nic3 gene, Nic1 ERF gene or Nic2 ERF gene, includes the native nucleotide sequence when in its natural environment and when it is linked to its naturally associated sequence(s) that is/are also in its/their natural environment. For ease of reference, we shall call this preferred embodiment the “native nucleotide sequence”. In this regard, the term “native nucleotide sequence” means an entire nucleotide sequence that is in its native environment and when operatively linked to an entire promoter with which it is naturally associated, which promoter is also in its native environment.

A nucleotide sequence encoding either a protein which has the specific properties as a Nic3 gene, Nic1 ERF or a Nic2 ERF as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

In a yet further alternative, the nucleotide sequence encoding the Nic3 gene or the Nic1 ERF or Nic2 ERF may be prepared synthetically by established standard methods, e.g. the phosphoroamidite method described by Beucage S. L. et al., (1981) Tetrahedron Letters 22, p 1859-1869 which is incorporated herein by reference, or the method described by Matthes et al., (1984) EMBO J. 3, p 801-805 which is incorporated herein by reference. In the phosphoroamidite method, oligonucleotides are synthesised, e.g. in an automatic DNA synthesiser, purified, annealed, ligated and cloned in appropriate vectors.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The present invention also encompasses the use of sequences having a degree of sequence identity or sequence homology with amino acid sequence(s) of a polypeptide having the specific properties defined herein or of any nucleotide sequence i.e. Nic3 gene, Nic1 ERF gene, Nic2 ERF gene encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence and/or fragments should provide and/or encode a polypeptide which retains the functional activity and/or enhances the activity of the Nic3 or Nic1 ERF or Nic2 ERF gene. Typically, the homologous sequences will comprise the same active sites etc. as the subject amino acid sequence for instance or will encode the same active sites. Although homology can also be considered in terms of similarity (i.e. amino acid residues having similar chemical properties/functions), in the context of the present invention it is preferred to express homology in terms of sequence identity. Homologous sequences typically retain functional domains or motifs.

In one embodiment, a homologous sequence is taken to include an amino acid sequence or nucleotide sequence which has one, two or several additions, deletions and/or substitutions compared with the subject sequence.

Homology or identity comparisons can be conducted by eye, or more usually, with the aid of readily available sequence comparison programs. These commercially available computer programs can calculate % homology between two or more sequences. % homology or % identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al. 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.qov), FASTA (Altschul et al. 1990 J. Mol. Biol. 403-410) and AlignX for example. At least BLAST, BLAST 2 and FASTA are available for offline and online searching (see Ausubel et al. 1999, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result.

Should gap penalties be used when determining sequence identity, then preferably the following parameters are used for pairwise alignment:

| FOR BLAST | | | |
|---|---|---|---|
| GAP OPEN | 0 | | |
| GAP EXTENSION | 0 | | |
| FOR CLUSTAL | DNA | PROTEIN | |
| WORD SIZE | 2 | 1 | K triple |
| GAP PENALTY | 15 | 10 | |
| GAP EXTENSION | 6.66 | 0.1 | |

In one embodiment, CLUSTAL may be used with the gap penalty and gap extension set as defined above. In some embodiments the gap penalties used for BLAST or CLUSTAL alignment may be different to those detailed above. The skilled person will appreciate that the standard parameters for performing BLAST and CLUSTAL alignments may change periodically and will be able to select appropriate parameters based on the standard parameters detailed for BLAST or CLUSTAL alignment algorithms at the time.

Suitably, the degree of identity with regard to a nucleotide sequence may be determined over at least 50 contiguous nucleotides, preferably over at least 60 contiguous nucleotides, preferably over at least 70 contiguous nucleotides, preferably over at least 80 contiguous nucleotides, preferably over at least 90 contiguous nucleotides, preferably over at least 100 contiguous nucleotides, preferably over at least 150 contiguous nucleotides, preferably over at least 200 contiguous nucleotides, preferably over at least 250 contiguous nucleotides, preferably over at least 300 contiguous nucleotides, preferably over at least 350 contiguous nucleotides, preferably over at least 400 contiguous nucleotides, preferably over at least 450 contiguous nucleotides, preferably over at least 500 contiguous nucleotides, preferably over at least 550 contiguous nucleotides, preferably over at least 600 contiguous nucleotides, preferably over at least 650 contiguous nucleotides, or preferably over at least 700 contiguous nucleotides.

Suitably, the degree of identity with regard to a nucleotide, cDNA, cds or amino acid sequence may be determined over the whole sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 4 below. Amino acids in the same block in the second column and preferably in the same line in the third column may be substituted for each other:

TABLE 4
Examples of conservative substitutions

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, ß-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid # and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, which will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention. The present invention also encompasses sequences that are complementary to the nucleic acid sequences of the present invention or sequences that are capable of hybridising either to the sequences of the present invention or to sequences that are complementary thereto. The term “hybridisation” as used herein shall include “the process by which a strand of nucleic acid joins with a complementary strand through base pairing” as well as the process of amplification as carried out in polymerase chain reaction (PCR) technologies.

The present invention also relates to nucleotide sequences that can hybridise to the nucleotide sequences of the present invention (including complementary sequences of those presented herein). Preferably, hybridisation is determined under stringency conditions (e.g. 50° C. and 0.2×SSC {1×SSC=0.15 M NaCl, 0.015 M Na₃citrate pH 7.0}). More preferably, hybridisation is determined under high stringency conditions (e.g. 65° C. and 0.1×SSC {1×SSC=0.15 M NaCl, 0.015 M Na₃citrate pH 7.0}).

In one aspect the sequence for use in the present invention is a synthetic sequence—i.e. a sequence that has been prepared by in vitro chemical or enzymatic synthesis. It includes, but is not limited to, sequences made with optimal codon usage for host organisms.

The term “expression vector” means a construct capable of in vivo or in vitro expression. In one embodiment the vector of the present invention expresses a Nic3 gene as described herein. In one embodiment the vector of the present invention further expresses a Nic1 ERF and/or aNic2 ERF gene as described herein. Preferably, the expression vector is incorporated into the genome of a suitable host organism. The term “incorporated” preferably covers stable incorporation into the genome.

The nucleotide sequence for use in the present invention may be present in a vector in which the nucleotide sequence is operably linked to regulatory sequences capable of providing for the expression of the nucleotide sequence by a suitable host organism. The constructs for use in the present invention may be transformed into a suitable host cell as described herein to provide for expression of a polypeptide of the present invention. The choice of vector e.g. a plasmid, cosmid, or phage vector will often depend on the host cell into which it is to be introduced. Vectors may be used in vitro, for example for the production of RNA or used to transfect, transform, transduce or infect a host cell.

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of a Nic3 gene as described herein operably linked to such a regulatory sequence, i.e. the vector is an expression vector. Suitably the vector may additionally comprise the nucleotide sequence of a Nic1 ERF gene and/or a Nic2 ERF gene as described herein is operably linked to a regulatory sequence.

The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals. The term “promoter” is used in the normal sense of the art, e.g. an RNA polymerase binding site. The nucleotide sequence within a construct which encodes a Nic3 gene or a Nic3 gene in combination with a Nic1 ERF gene and/or a Nic2 ERF gene may be operably linked to at least a promoter.

The term “construct”—which is synonymous with terms such as “cassette” or “vector”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment. The construct may even contain or express a marker, which allows for the selection of the genetic construct.

A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27), which are incorporated herein by reference. Further teachings on plant transformation may be found in EP-A-0449375, incorporated herein by reference.

In one embodiment provided herein are SNPs for use in genotyping plants (e.g. tobacco plants) having a low alkaloid (e.g. low nicotine) trait. Suitably, SNPs may be selected from tables 5-9 below. Suitably at least two SNPs may be selected, a first SNP may be selected from any of Tables 5 to 7 and a second SNP may be selected from any of Tables 5 to 7. Suitably at least two SNPs may be selected, a first SNP may be selected from Table 5 and a second SNP may be selected from Table 5. Suitably at least two SNPs may be selected, a first SNP may be selected from Table 6 and a second SNP may be selected from Table 6. Suitably at least two SNPs may be selected, a first SNP may be selected from Table 7 and a second SNP may be selected from Table 7.

In one embodiment provided herein are markers for use in genotyping plants (e.g. tobacco plants) having a low alkaloid (e.g. low nicotine trait).

In one embodiment provided herein are SNPs for use in genotyping the Nic3 locus in plants (e.g. tobacco plants). Suitably, SNPs may be selected from tables 5-7 below.

In one embodiment provided herein are markers for use in genotyping the Nic3 locus in plants (e.g. tobacco plants).

TABLE 5
SNPs associated with the QTL identified in the Examples.

| | | SEQ | | |
|---|---|---|---|---|
| Landmark\|pos | Flanking_seq | ID No. | Variant | iupac |
| Nitab4.5_0000 | TACCAAAAAGAATTACATTTGATGTAAAGAGGATTACACTGA | 313 | [T/A] | W |
| 535\|228474 | ACAACTAC[T/A]TATTTTCGTTTATTAGAAAATGGACTTTCAT | | | |
| | TCATTATCATTAGTACACA | | | |
| Nitab4.5_0000 | TTGTAGAGAGGGGATTTTTCTGATAAGCATATGCTATATTCT | 314 | [C/A] | M |
| 535\|242933 | ACCAAAAG[C/A]TCAATAATACTTTACTTTTCCTGCTTATTTG | | | |
| | CATTGTTCTTACTGACTCT | | | |
| Nitab4.5_0000 | GTGAGACAGTTGTGTAATTGAATTGATCCATGCATCTAATAC | 315 | [C/T] | Y |
| 535\|243206 | TGACTTGT[C/T]TGATTCTTTGTTCTTATCAAAATCATTAAAA | | | |
| | ATGGTAGATAATGGTGTTA | | | |
| Nitab4.5_0000 | TTCCATCAGCGATACCCGCAATAAGGGGAGCGAGGCCACGC | 316 | [G/A] | R |
| 535\|243346 | TGGTCCACG[G/A]CAGCCGATATCCGCGACATGTTTGGGAG | | | |
| | GCGACTCCTGATTATGCTGAAG | | | |
| Nitab4.5_0000 | CAACAACTTGGGCGGATGTTCACAACTGGTACAAATCTAAG | 317 | [G/A] | R |
| 535\|244324 | ATAAAAATT[G/A]AAGATGATCAGCTCGGTTTCCCAGCGTCA | | | |
| | TCCGAGGGTCGGGTCCGGGAG | | | |
| Nitab4.5_0000 | AGAATCTTATCAGGTATGGAGATATTTTCAGGTACCAAAAGA | 318 | [G/A] | R |
| 535\|245534 | AAACGGAC[G/A]CAACAAAGTCCACAGGGAAGAACTTGAAC | | | |
| | AAGTCGCATAGTTTGAAAAGT | | | |
| Nitab4.5_0000 | TTCATGCGACGTCATGGTACGTAGGCAATCTACATAGAGTTC | 319 | [C/T] | Y |
| 535\|250082 | GATCGAAT[C/T]ATTTTTTTATTTATTAAAAGAAAAAAAGGA | | | |
| | AAAAAGAAAAACAGATAAAA | | | |
| Nitab4.5_0000 | TTTCATTTTGATGTAATAACAGGATCGCAAACCGGAACCTCG | 320 | [G/A] | R |
| 535\|255470 | ACGGAACA[G/AJCACCTCAATCGGCTCTCCACCTCGGTAAAC | | | |
| | TCCGTCACTCTTCCTACTTC | | | |
| Nitab4.5_0000 | TCCAGCCTTGTTTCCGGTTTCCTTGCTGAGTTCTGCTCGAGTT | 321 | [C/T] | Y |
| 535\|256906 | TCAAGTT[C/T]GAGGTGTTGTCCGAGTTTAAGGTCATGAACA | | | |
| | GTGATTAAAGTGTGGGTTT | | | |
| Nitab4.5_0000 | GCAACAGAACAACGACGAGCTTGCCAGAAAATGAGCTGAAA | 322 | [G/A] | R |
| 535\|263907 | AGACCACCA[G/A]CGGCTCCTTATCCACCTCGTCGACCAACG | | | |
| | ACCTTTCAGCCTGTTCTTTTT | | | |
| Nitab4.5_0000 | CTAGATACACACAGCCGCACACTTGTCTTCTTCGGTACTCCCT | 323 | [G/A] | R |
| 535\|264016 | CAACAAC[G/A]TCACACGCACACATACGAACAGAGGCGGAA | | | |
| | AGGGAAGGTTTAAAAAATCA | | | |
| Nitab4.5_0000 | CTGAGTTCTGCTCGAGTTTCAAGTTCGAGGTGTTGTCCGAGT | 324 | [T/A] | W |
| 535\|264309 | TTAAGGTC[T/A]TGAACAGTGATTAAAGTGTGGGTTTGAATC | | | |
| | CGTGTTTGGTTGAGTTTCCG | | | |
| Nitab4.5_0000 | ATCACATCATCACCCTATCATCCGAGCATAAACGGCCAAGCA | 325 | [G/A] | R |
| 535\|268652 | GAATCAAC[G/A]AACAAGGTGATTATACAAAATCTCAAAAA | | | |
| | GAGATTGGAAGAAGCTAAAGG | | | |
| Nitab4.5_0000 | ATCTCTTATAAAAATCCTCACGTTAAAGGGTTGATTTCGGAA | 326 | [G/A] | R |
| 535\|270041 | GAACTTAT[G/A]CCTGGAATCCAAATGTTCTTAGAGGGAAAT | | | |
| | ACACCCGAAATCTTGCATAA | | | |
| Nitab4.5_0000 | AGGAAGAGAAGCATCTACGCCCCTAGAAGAAATGGGTGGA | 327 | [A/G] | R |
| 535\|270356 | TCCTCCCCCG[A/G]TTCGATATTTTGGCCTTCGTCATTTTAGT | | | |
| | CTTCAGCATCATCGCCTTCCG | | | |
| Nitab4.5_0000 | CCGATTTCTCTTCGGTTCCTGAGAAATCACAACCGGAACCAG | 328 | [G/A] | R |
| 535\|270454 | AAGAATTG[G/A]TAGCATCAGGTCGAGCCGAAAGACCCCTT | | | |
| | TTTGCAGCTAACTCCAACTCA | | | |
| Nitab4.5_0000 | CGAGCCGAAAGACCCCTTTTTGCAGCTAACTCCAACTCACGG | 329 | [G/T] | K |
| 535\|270516 | GCCTTAGA[G/T]ATTTCGGCATCAAATCAATAATACCTGCTTT | | | |
| | GGCCTATTCTAAGGTTTTT | | | |
| Nitab4.5_0000 | GGGGAAGAGGTACGTCGACCTTTCGGGAAACTGCGATCGGT | 330 | [G/A] | R |
| 535\|271398 | AGAGATGAT[G/A]CAGTAATTACTGGTGGCAGAAACAGAGT | | | |
| | TGGTGAAGAAGGAGATGAAGTT | | | |
| Nitab4.5_0000 | GCTAGTCCTTTCCACGTGCGGGTTCTGGGTGTAGACATGTGA | 331 | [C/A] | M |
| 535\|272053 | TTTTTGAC[C/A]CTCCCCAAGATTTTTCATAAGCATGTAAATA | | | |
| | TTTAATTTAGGCCTAATAT | | | |
| Nitab4.5_0000 | ACCAGATGAGCCTCCCCAATGCCACTTCTCTCCACAGAACTC | 332 | [G/A] | R |
| 535\|279401 | CCAAAGTC[G/A]TACATCACTCTTGCAGTACCCAGCTCCACA | | | |
| | AAATGCCTATCTACCCCCAC | | | |
| Nitab4.5_0000 | GTAACACTCTAGTCCAAGCAGGAAAAATAATGAAAATGAGA | 333 | [C/T] | Y |
| 535\|281378 | GAGTCTTAT[C/T]GGTGAAAACCCTTACAGGCACCGTAAGGC | | | |
| | GAAGGGAGCTGAGAGAAATCA | | | |
| Nitab4.5_0000 | ACTTTATAGTTTTCTCGTTAGGAATCATCTTTTTCCATTGTCCT | 334 | [C/A] | M |
| 535\|281825 | TCACTC[C/A]TTTCCTTTTTGTCTTGTTTACTTCCTTTTCCTAAG | | | |
| | TCTGTGCAGTTAGAA | | | |
| Nitab4.5_0000 | GCTAACACATAAAATGTCATAAGTCAGAAAAGAACAACAGG | 335 | [C/T] | Y |
| 535\|281986 | TGCATTGAG[C/T]TGGTAATAATCAACATGCTTTGGGATCCT | | | |
| | GTGAAAATCAAAGGTTGGTAC | | | |
| Nitab4.5_0000 | AAGTTATCATGGCAAGTGAAGGAGCAATAGACCTCAAGGCC | 336 | [G/A] | R |
| 535\|282301 | AATGCTAGT[G/A]GGTTAAGTCAGGAAATAACAACATGCAC | | | |
| | ATTGAGTGGATGGTAATTGACG | | | |
| Nitab4.5_0000 | TGGATAATGGGATTTAGTTTTTAAATTTTCAGGACTCTTCTAG | 337 | [G/A] | R |
| 535\|283098 | ATAATGG[G/A]ATTTAATTTTAAAAAAGTTTTCAAGCTTCTG | | | |
| | GTTAATGGGATTTAGTTTT | | | |
| Nitab4.5_0000 | GTTAAAATCGAACCACAGTCTAAAATATGTACTAAAATCAGA | 338 | [C/T] | Y |
| 535\|286896 | TAATAAGC[C/T]GAATATAGYAGTTGAGCGACCGTGCTAGAA | | | |
| | CCACGGAACTCGGGAATGCC | | | |
| Nitab4.5_0000 | GAACCACAGTCTAAAATATGTACTAAAATCAGATAATAAGCY | 339 | [T/C] | Y |
| 535\|286905 | GAATATAG[T/C]AGTTGAGCGACCGTGCTAGAACCACGGAA | | | |
| | CTCGGGAATGCCTAACACCTT | | | |
| Nitab4.5_0000 | GGGTAAGGTTATCCGCTCTGCCAGATCAGGAGCCATAATAT | 340 | [G/T] | K |
| 535\|287784 | CTAAATCTT[G/T]GCAATGACAGTCTTTTCCACAACAGGAAT | | | |
| | ACTAGAAGGACAGATGGAAGA | | | |
| Nitab4.5_0000 | TACAACGATAACCGGCAAAAGTGATGCTAAATCAAGGGATG | 341 | [T/C] | Y |
| 535\|288224 | ACGCGTGTT[T/C]AGCCCCAACCCACTCATGGCTGCATCTGC | | | |
| | GATAGACAACTCGCTTCTGCG | | | |
| Nitab4.5_0000 | AAGAAATTTCAGATTTTTCCTAAGTCCATATTTTTATCCGTTA | 342 | [G/A] | R |
| 535\|288360 | ACCATCC[G/A]AACTTCGCCGGAGGCCCTCGGTACCTCAACC | | | |
| | AAACATACCAAAAATCCAA | | | |
| Nitab4.5_0000 | TTATCATTATATTGTGGTTTATTGTTAATTGTCTTACCTAACG | 343 | [C/A] | M |
| 535\|289102 | GATTGGG[C/A]TATGTGCCATCACGACTAGTTGAATTTTGGG | | | |
| | TCGTAACAAGTTGGTATCA | | | |
| Nitab4.5_0000 | TGCAAGATGTGTTCAGATGGTTTGAATGTAACTGGAAGAGT | 344 | [A/G] | R |
| 535\|289649 | TTGCTTGAG[A/G]TGTGAAAAATGACTATTGGATGCTCGATT | | | |
| | TATGTGGATGTGTCATATAGT | | | |
| Nitab4.5_0000 | GGATGCAATCTTTAACCATTAATTTTTAGGTTTCAAGTTCGAG | 345 | [A/G] | R |
| 535\|290239 | ACATAAG[A/G]ATTGAGAAAATCCTTTACAATGAGATTTTCT | | | |
| | CTTTAGCAAGCCTCGAGGT | | | |
| Nitab4.5_0000 | TAGCTTCGTATTATATTTTTTTCCCATGGAGCTAGAAATAATT | 346 | [G/A] | R |
| 535\|295882 | AAATAAG[G/A]TATATATATTTTTGATCATTTGGATATAACCA | | | |
| | TGATACCTCGTTACTTCC | | | |
| Nitab4.5_0000 | AGTGCATAAAAGTGACAACAAAGACAACAAACCCATTGAAT | 347 | [G/A] | R |
| 535\|296545 | TCCACAAAT[G/A]GGGTCTGGAGAGGATAGTGTATACGCAG | | | |
| | ACCTTACCCTTACCTTATGAAG | | | |
| Nitab4.5_0000 | ATTAATACGAGTATCATGGTAAATTATGTACGTTATTTATTAT | 348 | [G/T] | K |
| 535\|299417 | TTCTTAA[G/T]GGACGTAAAAAGTCAAAACGTGACATGTAAA | | | |
| | AATGAACGGAGGGAGTACT | | | |
| Nitab4.5_0000 | CACTTCATCCACCCCACCCCTATACGGTGTGGCTTCCTCGTCG | 349 | [C/T] | Y |
| 535\|300790 | ATCTCCC[C/T]GATCCCCTGAATAACCGACCCAAGGTACTTG | | | |
| | AAACTACCCCTCTTGGGAA | | | |
| Nitab4.5_0000 | GCGGACCCATAATTTTTAGGCGATGGGGCACATTATTCTGCT | 350 | [G/C] | S |
| 535\|301472 | CAACCAAT[G/C]CCCCCTAAAGGCTTTTAGTGTTCAGGGTGC | | | |
| | CAACTGCTTTAAATTAATTA | | | |
| Nitab4.5_0000 | AGATTGTACATCCTCCTTATTTTTGGTAATCTTGTCACCTTCAC | 351 | [C/T] | Y |
| 535\|311720 | CCTCCC[C/T]CTTCTCTTGAGAAACCTTTTTCTTTTCTAGAGCC | | | |
| | CTACAATTGACTATGT | | | |
| Nitab4.5_0000 | GCCCCGCCAGCCACGCCGCCAATCTGCCGGAAAACACAGTT | 352 | [C/T] | Y |
| 535\|312924 | CCCATCCAT[C/T]GAGCAGCCCATCTTCTTCTTCAGCTGAACG | | | |
| | ACCAAACGACCCCTGACCTC | | | |
| Nitab4.5_0000 | GTGCGTAGTTGCTTTAGGCGTGCTATTTTAAAATTACCTTCCT | 353 | [G/A] | R |
| 535\|314236 | AAACTCG[G/A]GTGTGCATTTATGTGACCAAATCCAAGTCTC | | | |
| | AACAACGTTAGATAAAATG | | | |
| Nitab4.5_0000 | CCGATCTTCTAGAACTACGCAAAGATCTGATTCATGCGAGGT | 354 | [G/A] | R |
| 535\|316783 | CATGATAC[G/A]TAGGCAACCTACATAGGGTTCGATCGAATC | | | |
| | ATTTTTTTATCACTAAAAGA | | | |
| Nitab4.5_0000 | AAAAAGGGACAAGAGGATCAAAGGCCATTGTTTGTTGCTAT | 355 | [G/A] | R |
| 535\|329120 | TGTGGTGCC[G/A]TTTAACATCAACATGGATCTGGCACGGTT | | | |
| | TTGGCGTTTTTTTTTTGTCAC | | | |
| Nitab4.5_0000 | TGTCACTTTAAGTTGGAAAAAACTCCCTTATATACTCCCTTCT | 356 | [G/A] | R |
| 535\|329994 | CGGGGGT[G/A]TAAGAAATAGGAGGTGTGACAAGTGTATCC | | | |
| | ATGTTGAATGCACCAGTTGA | | | |
| Nitab4.5_0000 | GCAATTAGGGAAAGAATTGTTGATGGCTATCTATAGAGTCC | 357 | [G/A] | R |
| 535\|330357 | AACTGATTA[G/A]CTGCTCCATTTTGAAAAGCTTAAATCTCTT | | | |
| | TTGATTCCCATACTTACAAG | | | |
| Nitab4.5_0000 | TCTGAACCAAATAAAGATTTTTGCTCTTTAATATACTCCATAT | 358 | [C/T] | Y |
| 535\|333529 | AATCTGA[C/T]TGCACTGATCCTAGATCCGATAATTTTGTCTC | | | |
| | ACCCATTACAAACTGCTC | | | |
| Nitab4.5_0000 | ATCACTGCACAGGGTACAATCATTGCAACATCAATGACCTAT | 359 | [C/T] | Y |
| 535\|336046 | CCTGAGTC[C/T]GAGCTCACTCTCTAGTTGCACCAGTCCATTC | | | |
| | ACTCCTCGTGGACATCAAT | | | |
| Nitab4.5_0000 | CCCCAAATCGCTACTAACTTTAAACCTTTCCGGGCATCATCTT | 360 | [C/T] | Y |
| 535\|336533 | TCTCGAG[C/T]CKTCAACATTAGAAACACTGACTCAATCCTGA | | | |
| | ACCACTGCACCCCGCGAT | | | |
| Nitab4.5_0000 | CCAAATCGCTACTAACTTTAAACCTTTCCGGGCATCATCTTTC | 361 | [G/T] | K |
| 535\|336535 | TCGAGYC[G/T]TCAACATTAGAAACACTGACTCAATCCTGAA | | | |
| | CCACTGCACCCCGCGATCT | | | |
| Nitab4.5_0000 | AAATACCAAGTCTCATTACTCCATCAACCCAAATTTGAACATT | 362 | [C/T] | Y |
| 535\|336771 | CTTAGTC[C/T]GATTGCCTTTCCCCTGCTGGGAATAAAATACC | | | |
| | AAATCTCTAGATCATGCA | | | |
| Nitab4.5_0000 | GGAAACATCCTACCATCATATCAATACTGCGGAACAATCGTT | 363 | [C/T] | Y |
| 535\|336916 | CATAGCAT[C/T]ATGGCAACTTGTGCCTAGCCTCAGAGCTCT | | | |
| | CAGAAGTACTGCTACTGAGC | | | |
| Nitab4.5_0000 | ATCAAATCGCACGATGAGGAATCAAGAAGGGAAGTACTCCT | 364 | [G/A] | R |
| 535\|337195 | AATAGCCCT[G/A]TAGCCTCCCGAGATAAGTACAGACATCTC | | | |
| | CTGTCACGATCCAAACCGAAG | | | |
| Nitab4.5_0000 | CCTCTGTGGGCATCATCATCATCATATTGTACTAGCCTCAAA | 365 | [A/G] | R |
| 535\|337944 | GAGGACTC[A/G]GTAAAAGCGTACCCGACCATCATAAGGTT | | | |
| | CAGTAGAATCGTACCCGGCCA | | | |
| Nitab4.5_0000 | CACCGCCTAAGTGGGTCCCATTGGGAGCTTCTTGCGCGGTCT | 366 | [C/T] | Y |
| 535\|342273 | CGCGAAAA[C/T]GCGAATATCTCTCTACTCTGATATCGTATTG | | | |
| | ATGAACGGTTTAATGCGTT | | | |
| Nitab4.5_0000 | CGATCGCGCTGCTAATACAAGAATCACCATACAGAGCTATTC | 367 | [A/G] | R |
| 535\|345340 | CCAAACTC[A/G]GAATCCCAAACGAACATCGATAACACTGAA | | | |
| | ATGCAATTCAACCCAAACTT | | | |
| Nitab4.5_0000 | CCGGAATTCAATTCCGACGATGCGCACAAGTCATAATACCCG | 368 | [T/C] | Y |
| 535\|345668 | AAGTGAAG[T/C]TGTTCATGGCCTCAAACCGCCGAAAAACAT | | | |
| | GCCAAAGCTCAAAACGACCG | | | |
| Nitab4.5_0000 | TAATTAGATTAGTGGACTGCCACGTTTCCCTTGTTAGGGGAT | 369 | [T/A] | W |
| 535\|346524 | TTAAATTT[T/A]ATCTAATAATTAATTAATTAGGTAATATYCC | | | |
| | GCTACCTGGTAATTAACCA | | | |
| Nitab4.5_0000 | CTTGTTAGGGGATTTAAATTTWATCTAATAATTAATTAATTA | 370 | [C/T] | Y |
| 535\|346553 | GGTAATAT[C/T]CCGCTACCTGGTAATTAACCAATTACCCGTA | | | |
| | AAATAAAAAAAATTGCCCC | | | |
| Nitab4.5_0000 | TTGTTGGTTGGAAGAATTGCCCCTTTGGCCCTGATAATTGTT | 371 | [G/A] | R |
| 535\|349363 | CACATATT[G/A]AACTTCTTCATTCTGCCCATCATAATCATCA | | | |
| | TCTTGAAATCCACTACAAT | | | |
| Nitab4.5_0000 | GAACTTATCCTCCAACTCATCCCAAGTAGTGATGGAATGGTT | 372 | [G/T] | K |
| 535\|349990 | GGGAAGTC[G/T]CTCAAGCCAATCTAATACCTTCCCCCCCTA | | | |
| | AGTGATAAAGGGAATAATCT | | | |
| Nitab4.5_0000 | ATACACTAAACTTCTCACTCTATACCTATCGGTAGTTTGAGTG | 373 | [C/G] | 5 |
| 535\|350939 | ATTTTGC[C/G]CTAATTGACTTTCTCAAGACCAATTGGGTATG | | | |
| | ATAATTTGTGCAAGCAAT | | | |
| Nitab4.5_0000 | TGATATCCTGGTGTACTCTTGTAGCAGAAGGAGCATGCCCAT | 374 | [G/A] | R |
| 535\|352791 | CATTTGCG[G/A]ATTGTATTGCAGYGGTTGAGAGAGGAGAA | | | |
| | ACTTTATGCTAAGTTCTCTAG | | | |
| Nitab4.5_0000 | TACTCTTGTAGCAGAAGGAGCATGCCCATCATTTGCGRATTG | 375 | [C/T] | Y |
| 535\|352804 | TATTGCAG[C/T]GGTTGAGAGAGGAGAAACTTTATGCTAAG | | | |
| | TTCTCTAGGTGTGAGTTTTGG | | | |
| Nitab4.5_0000 | GGAGTGGAAGTGAGAGCGGATCACCATGGATTTCATAGTTG | 376 | [G/A] | R |
| 535\|354051 | GGATCCCAC[G/A]GAACTCGAGGAGGTTCGATTTTGTTTGG | | | |
| | GTGATTGTGGATCGACTGACCA | | | |
| Nitab4.5_0000 | TATGAGCTTGCCTTACCACCTAGCTTGTCGGGGGTGCATCCA | 377 | [T/C] | Y |
| 535\|354819 | ATATTTCA[T/C]GTTTCTATGCTCYAGAAGTATATTAGCGATC | | | |
| | CGTCTCATGTTTTGAATTT | | | |
| Nitab4.5_0000 | TACCACCTAGCTTGTCGGGGGTGCATCCAATATTTCAYGTTTC | 378 | [T/C] | Y |
| 535\|354832 | TATGCTC[T/C]AGAAGTATATTAGCGATCCGTCTCATGTTTTG | | | |
| | AATTTCAGCACAGTTCAG | | | |
| Nitab4.5_0000 | TGGATGTGATTTGATAGGTTTTGGCATTGTTGGTAGAATTTG | 379 | [G/A] | R |
| 535\|355608 | GAAGTTTC[G/A]AGGTTCATTAGGCTTAAATCCGTGTGCAAG | | | |
| | TCGTGTTTTTGATGTTGTTC | | | |
| Nitab4.5_0000 | GGACGTGTTGGTATGTTTGGTTGAGGTCCCGAGGGCCTCGG | 380 | [G/A] | R |
| 535\|355749 | GTTGATTTC[G/A]GGTGGTTAACAGAATAAGATTGAAGTTTG | | | |
| | AAGGATTRCTGTAGTCTGGTG | | | |
| Nitab4.5_0000 | TCGGGTTGATTTCRGGTGGTTAACAGAATAAGATTGAAGTTT | 381 | [G/A] | R |
| 535\|355786 | GAAGGATT[G/A]CTGTAGTCTGGTGTTTTTGCACCTACGGTG | | | |
| | GGGAGCCAGCAGGTGCTGAC | | | |
| Nitab4.5_0000 | GCGAGGTCGTGTACTTGCTGAATTAGATGCTAATTGTTGTTT | 382 | [G/A] | R |
| 535\|356948 | TGTACTCA[G/A]CCATAGCTTTTTTTGCTTATTATGCCTCCGTC | | | |
| | TCTTATCATTGTTGAAAC | | | |
| Nitab4.5_0000 | CCGAGTGATTGAGCTATTGAGATGAGCTAAGTGATTGTTGC | 383 | [C/T] | Y |
| 535\|357398 | CTTGAGAGG[C/T]TGTACTTGCTTTCGGTTATTGTTGCACTAG | | | |
| | TTGCTATTTGCCCTTATTGT | | | |
| Nitab4.5_0000 | TTCACGACTTCAACATGGTAAGTATCATATATCAGAAAGTGA | 384 | [C/T] | Y |
| 535\|359956 | TTATATTT[C/T]ATAAATCCATGGTTATATTCATCGTTTAATTT | | | |
| | AGATTTAAATGGAAGAAA | | | |
| Nitab4.5_0000 | AGGAGCGATAAGGGTGGCTATTGATATTGTCAGGGCGGAG | 385 | [G/T] | K |
| 535\|360937 | GGATAAGAGA[G/T]GCTATTATATGAGTGATAAGGGTGTAT | | | |
| | ATATGAGCAATAAGGGTGGTTAT | | | |
| Nitab4.5_0000 | TTCTGCACATTTTGTGCAGAGCTAGGTACTAAAGCTTTCAGA | 386 | [T/G] | K |
| 535\|361967 | CTCGAGCA[T/G]AGTTAGTGTGTTGATTGCAAGGATTTAAGG | | | |
| | TAGAGTTGCTTGGTCGTCAT | | | |
| Nitab4.5_0000 | TGCAAAATATAGAAGTTCAAAGATTTGAAAACTTATAATTTG | 387 | [C/T] | Y |
| 535\|365559 | ATTCGATG[C/T]GCGATTCATAATTTCGACATTTTTTGATGTG | | | |
| | ATTTGAAGTATCGACTAAG | | | |
| Nitab4.5_0000 | TGTGTTTGGATTTCATGAAAATTATGTAGGGTTCTGAGAGGC | 388 | [C/T] | Y |
| 535\|366358 | GAGTCCCG[C/T]GTTGACTTGTGGTTGACCTTTTTGTAACGA | | | |
| | CCCGACCATTTATTTGAGAG | | | |
| Nitab4.5_0000 | GATTGCATTTTGGATGTCTGTAGCTCATTTAAACTTGAAATG | 389 | [C/T] | Y |
| 535\|366691 | GCGAAAGT[C/T]GAATTTTTGGAGTTTTGGTCCGATAGTAGA | | | |
| | ATTTTTGATATCAGGGTCGG | | | |
| Nitab4.5_0000 | TGCGGTCTCAGCCCATTTTAAATTAATTTCGCACCTGCGATG | 390 | [G/A] | R |
| 535\|367265 | GTTTTCCC[G/A]CAGGTGCGGGCCGCAAATACGTGTGAGTA | | | |
| | ACCGCATGTGCAGTTTAACTA | | | |
| Nitab4.5_0000 | TAGATTCTGGAGTATAGAATTACTGTTTCCTTTCTATAGTTTG | 391 | [C/T] | Y |
| 535\|368361 | TGACTTA[C/T]GACATTCCGGGTTTTGGGAGAGGTTATGTGC | | | |
| | ACCTCGAGAGTGGTTATTG | | | |
| Nitab4.5_0000 | TTCTGCATTGGTTTTTCGGCCGTCGAGGTCGAGCCGTGAAAT | 392 | [G/A] | R |
| 535\|372498 | CAATTACT[G/A]ATGTATCAGAATAGGTTGCTTACATTATATC | | | |
| | TTTTGGAATGTGATTTTAT | | | |
| Nitab4.5_0000 | AAAAGTCAGTTTTGATAAAAATACATTGTGTAGAGGCGTAG | 393 | [A/G] | R |
| 535\|374002 | CAAGATTTT[A/G]CACTTTATAAGTTCTAAATTTAAAATAGCG | | | |
| | ACACCAGTTATTAATAATTG | | | |
| Nitab4.5_0000 | TAGTAAATTATTGTTATATACAGATGCTGTTATAGAGAAGTC | 394 | [C/T] | Y |
| 535\|374421 | TGATTGTT[C/T]TAATTATTTATTCATAATAGCAAACATCAGC | | | |
| | TATGTTTGGCTTTTTAAAA | | | |
| Nitab4.5_0000 | GTAACTAAGATAAGCAGTGGATTCAATAATAAATTTAGAACC | 395 | [T/C] | Y |
| 535\|375362 | CATAAACT[T/C]CATATCCTAGCTCCGGCACCGAGCAGCTCA | | | |
| | AAATATTACCATTTTAAAAA | | | |
| Nitab4.5_0000 | TACCAAAAAGAATTACATTTGATGTAAAGAGGATTACACTGA | 396 | [T/A] | W |
| 535\|228474 | ACAACTAC[T/A]TATTTTCGTTTATTAGAAAATGGACTTTCAT | | | |
| | TCATTATCATTAGTACACA | | | |
| Nitab4.5_0000 | CCTTCTTCTACTCCCCTTTCTCCCCTACTCGTCGACTTAGCTGC | 397 | [T/G] | K |
| 535\|236506 | TCGATT[T/G]GAATTTGGTTTGTTTGAGACCTCAATGATCACT | | | |
| | TCATTTTCCGATCCTTT | | | |
| Nitab4.5_0000 | TTGTAGAGAGGGGATTTTTCTGATAAGCATATGCTATATTCT | 398 | [C/A] | M |
| 535\|242933 | ACCAAAAG[C/A]TCAATAATACTTTACTTTTCCTGCTTATTTG | | | |
| | CATTGTTCTTACTGACTCT | | | |
| Nitab4.5_0000 | AGAATCTTATCAGGTATGGAGATATTTTCAGGTACCAAAAGA | 399 | [G/A] | R |
| 535\|245534 | AAACGGAC[G/A]CAACAAAGTCCACAGGGAAGAACTTGAAC | | | |
| | AAGTCGCATAGTTTGAAAAGT | | | |
| Nitab4.5_0000 | ACCTGGGAATGCCTAACACCTTCTCCCGTGTTAATAAAATTC | 400 | [G/A] | R |
| 535\|247977 | CTTGCCCG[G/A]ATTTCTGTGTTCGCAAACCATAATTAGAGTT | | | |
| | AAACCTTCCTCGATGCTAG | | | |
| Nitab4.5_0000 | AGCTAGCACTTGCAACTAGCAAGCGTCAAGGTTCCAATCCAA | 401 | [T/A] | W |
| 535\|263142 | AGTCTACA[T/A]GAAGAACCATTCAAGACTCAAGATCAAGCT | | | |
| | TCAGAAGACTTATAGATAGG | | | |
| Nitab4.5_0000 | CTGAGTTCTGCTCGAGTTTCAAGTTCGAGGTGTTGTCCGAGT | 402 | [T/A] | W |
| 535\|264309 | TTAAGGTC[T/A]TGAACAGTGATTAAAGTGTGGGTTTGAATC | | | |
| | CGTGTTTGGTTGAGTTTCCG | | | |
| Nitab4.5_0000 | ATCTCTTATAAAAATCCTCACGTTAAAGGGTTGATTTCGGAA | 403 | [G/A] | R |
| 535\|270041 | GAACTTAT[G/A]CCTGGAATCCAAATGTTCTTAGAGGGAAAT | | | |
| | ACACCCGAAATCTTGCATAA | | | |
| Nitab4.5_0000 | AGGAAGAGAAGCATCTACGCCCCTAGAAGAAATGGGTGGA | 404 | [A/G] | R |
| 535\|270356 | TCCTCCCCCG[A/G]TTCGATATTTTGGCCTTCGTCATTTTAGT | | | |
| | CTTCAGCATCATCGCCTTCCG | | | |
| Nitab4.5_0000 | TTCTAACCACTTTTTGGCCAAATAAGTCATTTTGCAAAAATAG | 405 | [C/T] | Y |
| 535\|275825 | CCTCAAT[C/T]CTGTCTTGCATGTGTCTAGTAGGGAATGTTAT | | | |
| | TGTCTCACATAGTATTGG | | | |
| Nitab4.5_0000 | GTAACACTCTAGTCCAAGCAGGAAAAATAATGAAAATGAGA | 406 | [C/T] | Y |
| 535\|281378 | GAGTCTTAT[C/T]GGTGAAAACCCTTACAGGCACCGTAAGGC | | | |
| | GAAGGGAGCTGAGAGAAATCA | | | |
| Nitab4.5_0000 | GCTAACACATAAAATGTCATAAGTCAGAAAAGAACAACAGG | 407 | [C/T] | Y |
| 535\|281986 | TGCATTGAG[C/T]TGGTAATAATCAACATGCTTTGGGATCCT | | | |
| | GTGAAAATCAAAGGTTGGTAC | | | |
| Nitab4.5_0000 | GTTAAAATCGAACCACAGTCTAAAATATGTACTAAAATCAGA | 408 | [C/T] | Y |
| 535\|286896 | TAATAAGC[C/T]GAATATAGYAGTTGAGCGACCGTGCTAGAA | | | |
| | CCACGGAACTCGGGAATGCC | | | |
| Nitab4.5_0000 | GAACCACAGTCTAAAATATGTACTAAAATCAGATAATAAGCY | 409 | [T/C] | Y |
| 535\|286905 | GAATATAG[T/C]AGTTGAGCGACCGTGCTAGAACCACGGAA | | | |
| | CTCGGGAATGCCTAACACCTT | | | |
| Nitab4.5_0000 | TACAACGATAACCGGCAAAAGTGATGCTAAATCAAGGGATG | 410 | [T/C] | Y |
| 535\|288224 | ACGCGTGTT[T/C]AGCCCCAACCCACTCATGGCTGCATCTGC | | | |
| | GATAGACAACTCGCTTCTGCG | | | |
| Nitab4.5_0000 | AATGTATTTTTTTGTTTTGGTGATGGGCCAGTCTAGAGGACT | 411 | [G/T] | K |
| 535\|289459 | TGAGGCAC[G/T]GTTTGGACCTTAGCTTAGGCTCTATAGTTT | | | |
| | CAGTTGTGTGAGCATGTGCT | | | |
| Nitab4.5_0000 | TGCAAGATGTGTTCAGATGGTTTGAATGTAACTGGAAGAGT | 412 | [A/G] | R |
| 535\|289649 | TTGCTTGAG[A/G]TGTGAAAAATGACTATTGGATGCTCGATT | | | |
| | TATGTGGATGTGTCATATAGT | | | |
| Nitab4.5_0000 | GGATGCAATCTTTAACCATTAATTTTTAGGTTTCAAGTTCGAG | 413 | [A/G] | R |
| 535\|290239 | ACATAAG[A/G]ATTGAGAAAATCCTTTACAATGAGATTTTCT | | | |
| | CTTTAGCAAGCCTCGAGGT | | | |
| Nitab4.5_0000 | TAATATTAAGTTCGTAGAATAGTTTCAGTTTATTTGTTGAAAA | 414 | [A/T] | W |
| 535\|296059 | CTAATTT[A/T]AAAAAAAGAAGAGTAAGAGATTATTCTTTTA | | | |
| | ATCTAGTGCGAATATTAAT | | | |
| Nitab4.5_0000 | TCATCAGGGCTTACCTCAACATTTCATTAATTGTTGCTAGAGC | 415 | [G/A] | R |
| 535\|302118 | TGCCAGA[G/A]ATAGAGTCGAGATTTGAAACTTATGGGTTCA | | | |
| | GAATTCTAGTCTTTTTAAG | | | |
| Nitab4.5_0000 | AGATTGTACATCCTCCTTATTTTTGGTAATCTTGTCACCTTCAC | 416 | [C/T] | Y |
| 535\|311720 | CCTCCC[C/T]CTTCTCTTGAGAAACCTTTTTCTTTTCTAGAGCC | | | |
| | CTACAATTGACTATGT | | | |
| Nitab4.5_0000 | CAAAAAAAAATTACTTTTATATTTCCTTTAGGATATTAGGACT | 417 | [C/A] | M |
| 535\|316145 | AAAAATT[C/A]AAAAAAAAAATATGATTTTTCTCTTTTGGTAT | | | |
| | CTCTTTACAAATTTTCTT | | | |
| Nitab4.5_0000 | ATTTTATTTTGCTTAGTTTGTTTAGTCATAATAGCCCGTTTAG | 418 | [T/A] | W |
| 535\|323584 | TGTTGTC[T/A]AGGTTTGTAACCCCAATTTAGTTTGTTTGTTTT | | | |
| | GTTGTCCAAATCCTTTC | | | |
| Nitab4.5_0000 | TCTGAACCAAATAAAGATTTTTGCTCTTTAATATACTCCATAT | 419 | [C/T] | Y |
| 535\|333529 | AATCTGA[C/T]TGCACTGATCCTAGATCCGATAATTTTGTCTC | | | |
| | ACCCATTACAAACTGCTC | | | |
| Nitab4.5_0000 | ATCAAATCGCACGATGAGGAATCAAGAAGGGAAGTACTCCT | 420 | [G/A] | R |
| 535\|337195 | AATAGCCCT[G/A]TAGCCTCCCGAGATAAGTACAGACATCTC | | | |
| | CTGTCACGATCCAAACCGAAG | | | |
| Nitab4.5_0000 | CGATCGCGCTGCTAATACAAGAATCACCATACAGAGCTATTC | 421 | [A/G] | R |
| 535\|345340 | CCAAACTC[A/G]GAATCCCAAACGAACATCGATAACACTGAA | | | |
| | ATGCAATTCAACCCAAACTT | | | |
| Nitab4.5_0000 | CCGGAATTCAATTCCGACGATGCGCACAAGTCATAATACCCG | 422 | [T/C] | Y |
| 535\|345668 | AAGTGAAG[T/C]TGTTCATGGCCTCAAACCGCCGAAAAACAT | | | |
| | GCCAAAGCTCAAAACGACCG | | | |
| Nitab4.5_0000 | TAATTAGATTAGTGGACTGCCACGTTTCCCTTGTTAGGGGAT | 423 | [T/A] | W |
| 535\|346524 | TTAAATTT[T/A]ATCTAATAATTAATTAATTAGGTAATATCCC | | | |
| | GCTACCTGGTAATTAACCA | | | |
| Nitab4.5_0000 | TACTCTTGTAGCAGAAGGAGCATGCCCATCATTTGCGGATTG | 424 | [C/T] | Y |
| 535\|352804 | TATTGCAG[C/T]GGTTGAGAGAGGAGAAACTTTATGCTAAG | | | |
| | TTCTCTAGGTGTGAGTTTTGG | | | |
| Nitab4.5_0000 | TACCACCTAGCTTGTCGGGGGTGCATCCAATATTTCATGTTTC | 425 | [T/C] | Y |
| 535\|354832 | TATGCTC[T/C]AGAAGTATATTAGCGATCCGTCTCATGTTTTG | | | |
| | AATTTCAGCACAGTTCAG | | | |
| Nitab4.5_0000 | GGCTCCAGGTATGTTTCTAGACCCATTCGAGGATGAACGTTT | 426 | [G/T] | K |
| 535\|355109 | GTTTAAGA[G/T]GGGGAGGATGTAACGACCCAGCCGGTCGT | | | |
| | TTCGAGAGTTATAGCCCCATT | | | |
| Nitab4.5_0000 | TGGATGTGATTTGATAGGTTTTGGCATTGTTGGTAGAATTTG | 427 | [G/A] | R |
| 535\|355608 | GAAGTTTC[G/A]AGGTTCATTAGGCTTAAATCCGTGTGCAAG | | | |
| | TCGTGTTTTTGATGTTGTTC | | | |
| Nitab4.5_0000 | TCCTTTAGCCTATATTGATTGTGTTGTACTACTTTTGGCTAGA | 428 | [C/T] | Y |
| 535\|356468 | TTCGGGA[C/T]GTTTGGAGGCCAGTTCGAAAGGCAAAGGCA | | | |
| | TTGCGGAGTAGGATTTTACT | | | |
| Nitab4.5_0000 | GTGAAAGACTGTATGGCCTTGTCGGCCTATGGTCAGTGTAC | 429 | [T/A] | W |
| 535\|359555 | GAGTGATCA[T/A]TTTGGTCTTATAGGCCAATATGTCTTATGT | | | |
| | ATAAGGTGGTATTACATATT | | | |
| Nitab4.5_0000 | TTCTGCACATTTTGTGCAGAGCTAGGTACTAAAGCTTTCAGA | 430 | [T/G] | K |
| 535\|361967 | CTCGAGCA[T/G]AGTTAGTGTGTTGATTGCAAGGATTTAAGG | | | |
| | TAGAGTTGCTTGGTCGTCAT | | | |
| Nitab4.5_0000 | GGGAGATTTGATGGTGTTAGCCTATGTCTATCATTTTGTTTT | 431 | [T/C] | Y |
| 535\|364083 | GTACACTA[T/C]TGAGGGTTATGAGTCCAAAAGTGACTTTCT | | | |
| | ATTACTCACTACAGTGGGTT | | | |
| Nitab4.5_0000 | GATTGCATTTTGGATGTCTGTAGCTCATTTAAACTTGAAATG | 432 | [C/T] | Y |
| 535\|366691 | GCGAAAGT[C/T]GAATTTTTGGAGTTTTGGTCCGATAGTAGA | | | |
| | ATTTTTGATATCAGGGTCGG | | | |
| Nitab4.5_0000 | CCCTTGTCCAGCTAGGCTGCAGGTGCAGTGAGGGATCCGCA | 433 | [G/A] | R |
| 535\|367169 | GAAGCATAA[G/A]CGCAGAAGTGGTTGGGGACCGCAAGTG | | | |
| | CGGCCTAGTGCTCGCAAATGCGG | | | |
| Nitab4.5_0000 | CATTCTGCATTGGTTTTTCGGCCGTCGAGGTCGAGCCGTGAA | 434 | [C/T] | Y |
| 535\|372496 | ATCAATTA[C/T]TRATGTATCAGAATAGGTTGCTTACATTATA | | | |
| | TCTTTTGGAATGTGATTTT | | | |
| Nitab4.5_0000 | TTCTGCATTGGTTTTTCGGCCGTCGAGGTCGAGCCGTGAAAT | 435 | [G/A] | R |
| 535\|372498 | CAATTAYT[G/A]ATGTATCAGAATAGGTTGCTTACATTATATC | | | |
| | TTTTGGAATGTGATTTTAT | | | |
| Nitab4.5_0000 | TAGTAAATTATTGTTATATACAGATGCTGTTATAGAGAAGTC | 436 | [C/T] | Y |
| 535\|374421 | TGATTGTT[C/T]TAATTATTTATTCATAATAGCAAACATCAGC | | | |
| | TATGTTTGGCTTTTTAAAA | | | |
| Nitab4.5_0000 | ACTAGAGCTGAAGTTCGCCAGTTACAAAAATAAAAACTTCAG | 437 | [G/C] | S |
| 535\|229285 | CACACTTG[G/C]TTCAGCACACTTGCTACTTCAGGTCCGTCTA | | | |
| | CTAGAATGCTGAAGTTTTA | | | |
| Nitab4.5_0000 | CGTATGCTGAAGTTATGTGAAAAAACGGGTACGCTTGCAAT | 438 | [C/T] | Y |
| 535\|229412 | TTGTTTTTG[C/T]AAAGCGGGCACAAGTTAAAACGTGACACA | | | |
| | AAAAGCGGATATAGATGTAAA | | | |
| Nitab4.5_0000 | CTTAAAGGAGCAATAGGAGGCCATAATATGTCATCCCACACT | 439 | [A/G] | R |
| 535\|243475 | GCAGGATC[A/G]GGTTATGACGGAATTAAAGCAAGCGTTAT | | | |
| | TGGGTGTATCCAATAATGAGA | | | |
| Nitab4.5_0000 | CGGCGCATGCTCCCAAGCCAAGCGAAGCTAACCAGGGGGA | 440 | [G/A] | R |
| 535\|245485 | GGAGTCGTCA[G/A]AATCTTATCAGGTATGGAGATATTTTCA | | | |
| | GGTACCAAAAGAAAACGGACGC | | | |
| Nitab4.5_0000 | ATAATTAGAGTTAAACCTTCCTCGATGCTAGATTTTAAACCG | 441 | [A/C] | M |
| 535\|248047 | GTGACTTG[A/C]GACACCAATAAACTATCCCAAGTGGCGAM | | | |
| | TCTGATTTTTATATGAATAAT | | | |
| Nitab4.5_0000 | AGATTTTAAACCGGTGACTTGMGACACCAATAAACTATCCC | 442 | [C/A] | M |
| 535\|248076 | AAGTGGCGA[C/A]TCTGATTTTTATATGAATAATCCCGTTTCG | | | |
| | ATTGTCACTTAAATTGAAAA | | | |
| Nitab4.5_0000 | TGAATGATGAATTGATTTTGTCATTTGAAAGTTAAACAAGTA | 443 | [G/T] | K |
| 535\|269931 | CTTCAAAT[G/T]CTAGTTCCGTAATGAACAGCAGAAGTCCTC | | | |
| | TTCAAGAGCACCGTAAACAT | | | |
| Nitab4.5_0000 | GGGGAAGAGGTACGTCGACCTTTCGGGAAACTGCGATCGGT | 444 | [G/A] | R |
| 535\|271398 | AGAGATGAT[G/A]CAGTAATTACTGGTGGCAGAAACAGAGT | | | |
| | TGGTGAAGAAGGAGATGAAGTT | | | |
| Nitab4.5_0000 | GCTAGTCCTTTCCACGTGCGGGTTCTGGGTGTAGACATGTGA | 445 | [C/A] | M |
| 535\|272053 | TTTTTGAC[C/AJCTCCCCAAGATTTTTCATAAGCATGTAAATA | | | |
| | TTTAATTTAGGCCTAATAT | | | |
| Nitab4.5_0000 | ATGTAAATTAAAGTGTAGTATTTATACATGTCCTAATAATTTC | 446 | [G/A] | R |
| 535\|274556 | GAATTTC[G/A]GATTGGATTGGATTAAAATATACCAATCCGA | | | |
| | AATCCGATCCGAAATCCGA | | | |
| Nitab4.5_0000 | GAGTCAATACCCCGATTGGTGAAGAAGAAATGGTTGAGCTT | 447 | [G/T] | K |
| 535\|279103 | TTCCTACAA[G/T]CCTAGGGTCCCACCTACTTTAGTCATTTGA | | | |
| | TCCCGGCCCTGGGGAAGCCT | | | |
| Nitab4.5_0000 | GGAAGAGTTTGCTTGAGATGTGAAAAATGACTATTGGATGC | 448 | [G/A] | R |
| 535\|289682 | TCGATTTAT[G/A]TGGATGTGTCATATAGTCTTGAGTTATAA | | | |
| | AGGTTTTTGAGAAATTCTTTC | | | |
| Nitab4.5_0000 | ATATATAATTCCAAATAAGTTTGAGGTCAGCTATATGAATAT | 449 | [C/G] | s |
| 535\|290818 | TTATTATT[C/G]ATATCGCTCTATTTAAACTTATTTAAATTAAA | | | |
| | TTAACTAATTTTATATTA | | | |
| Nitab4.5_0000 | ATCTTAGTTCGAATCCAAGACCTCTAATTAAGAGTAAAAGGA | 450 | [G/A] | R |
| 535\|295774 | TCCTATCC[G/A]TCACACTATAATCCTTGTTGATGGGAACCTC | | | |
| | TTTCTTCAACTCTCCTAAA | | | |
| Nitab4.5_0000 | TAGCTTCGTATTATATTTTTTTCCCATGGAGCTAGAAATAATT | 451 | [G/A] | R |
| 535\|295882 | AAATAAG[G/A]TATATATATTTTTGATCATTTGGATATAACCA | | | |
| | TGATACCTCGTTACTTCC | | | |
| Nitab4.5_0000 | TATATAATATATGCTAATTAAACACAGCTATTCATATATTATA | 452 | [G/A] | R |
| 535\|299003 | CATAAAC[G/A]TATATATTATACATAAACATATATATTATGCA | | | |
| | TTCCTCGTGTAAGTAGTT | | | |
| Nitab4.5_0000 | CTGCTTATGCTTTTTTGAGCCGAGAGTCTATTGGAAACAACC | 453 | [C/T] | Y |
| 535\|302771 | TCTCCATA[C/T]TCACAAAGTTGGGATAAGGTCTGCGTACAC | | | |
| | ACTACCCTCCCAGGCCCACG | | | |
| Nitab4.5_0000 | CATTCTCAGTATCACTGCCCCTTTTGTAGTTCATGTTGATTCT | 454 | [C/T] | Y |
| 535\|311059 | GTTCGTG[C/T]TGCCTTCGTTAACCTCTTGCTCCCATTCTTTTC | | | |
| | CATCCAAGTCGATAACT | | | |
| Nitab4.5_0000 | AAGAGGATCAAAGGCCATTGTTTGTTGCTATTGTGGTGCCAT | 455 | [T/G] | K |
| 535\|313843 | TTAACATC[T/G]ACATGGATCTGGCACGGTTTTGGTGTTTTTT | | | |
| | TTGTCACTTTGGTTTTGGT | | | |
| Nitab4.5_0000 | GTGCGTAGTTGCTTTAGGCGTGCTATTTTAAAATTACCTTCCT | 456 | [G/A] | R |
| 535\|314236 | AAACTCG[G/A]GTGTGCATTTATGTGACCAAATCCAAGTCTC | | | |
| | AACAACGTTAGATAAAATG | | | |
| Nitab4.5_0000 | TCTCATCTTCGGGGATGTGCTCGCTGGTCGAGACTCCTTATT | 457 | [G/A] | R |
| 535\|314848 | CTATTAGT[G/A]TCATACCTTGAAAGTAAGAAAGAGGCTCGG | | | |
| | ACAAGTTACTAAGCCGGATG | | | |
| Nitab4.5_0000 | AGTATGTTCTATCCTATGTGAATAAAATCTCAAAATGCATGG | 458 | [T/C] | Y |
| 535\|329401 | CCCTCACT[T/C]ATTTTTTTTAATCCTTAGAAATCGAGGCGTG | | | |
| | CCATTTAATTGAATTTCCA | | | |
| Nitab4.5_0000 | ACTACTAAAGATAACAATATCATCAGCATATGCCAAGTAATT | 459 | [G/A] | R |
| 535\|331807 | GATTTGAG[G/A]GCCCCTATGAGACATAGAGCAAGGTATAA | | | |
| | AATTATTATTGAGTTTCTAGT | | | |
| Nitab4.5_0000 | ATCACTGCACAGGGTACAATCATTGCAACATCAATGACCTAT | 460 | [C/T] | Y |
| 535\|336046 | CCTGAGTC[C/T]GAGCTCACTCTCTAGTTGCACCAGTCCATTC | | | |
| | ACTCCTCGTGGACATCAAT | | | |
| Nitab4.5_0000 | ACTTCAAACCTTTCTTAAGGTGCGTGGTCATCCTACCACAAG | 461 | [C/A] | M |
| 535\|336304 | CCCATATG[C/A]AACTCCGCCACTCTCCCACTTTGGCGAAACT | | | |
| | CACCCCTTTAGTCGACTAC | | | |
| Nitab4.5_0000 | CGCTGAGCTAATAGCCTACTGGAGGACTGTCAACCTATCAAT | 462 | [G/T] | K |
| 535\|337689 | CGAGACCT[G/T]CGGGCATGAAATGCAGCGTCCCCAGGCAA | | | |
| | AAGGGATGTCAGTACAAATAA | | | |
| Nitab4.5_0000 | CCTCTGTGGGCATCATCATCATCATATTGTACTAGCCTCAAA | 463 | [A/G] | R |
| 535\|337944 | GAGGACTC[A/G]GTAAAAGCGTACCCGACCATCATAAGGTT | | | |
| | CAGTAGAATCGTACCCGGCCA | | | |
| Nitab4.5_0000 | TTGTTGGTTGGAAGAATTGCCCCTTTGGCCCTGATAATTGTT | 464 | [G/A] | R |
| 535\|349363 | CACATATT[G/A]AACTTCTTCATTCTGCCCATCATAATCATCA | | | |
| | TCTTGAAATCCACTACAAT | | | |
| Nitab4.5_0000 | ATACACTAAACTTCTCACTCTATACCTATCGGTAGTTTGAGTG | 465 | [C/G] | S |
| 535\|350939 | ATTTTGC[C/G]CTAATTGACTTTCTCAAGACCAATTGGGTATG | | | |
| | ATAATTTGTGCAAGCAAT | | | |
| Nitab4.5_0000 | TCCCTAGTTATCAGGTCACTCAGCCCATTCTATCCCTCCATAC | 466 | [C/A] | M |
| 535\|352310 | CTTATGG[C/A]ACCTGCTGAGTTGAAAGAGTTGAATAAGTAG | | | |
| | CTTCAGGAACTCCTTGAGA | | | |
| Nitab4.5_0000 | AAATTTCCAAGATGTTAGATCGTGTTTAAAGTGAGGAAAGG | 467 | [C/T] | Y |
| 535\|363439 | GAGCCTAAG[C/T]AGGCCAAAAGGTCTCAAAATTCTGGAGG | | | |
| | ATTCGGTGGATTTTTTTTCTGCA | | | |
| Nitab4.5_0000 | CTTAGAATAGCATGCCTACTTACCTTAATTGCTTATCTGAGCT | 468 | [G/T] | K |
| 535\|367567 | CTGTGCA[G/T]CATGTTTAGTGAAATTTCCTGCTTTTCCTTGA | | | |
| | CTTGTACTTAGTCCACAG | | | |
| Nitab4.5_0000 | TAGATTCTGGAGTATAGAATTACTGTTTCCTTTCTATAGTTTG | 469 | [C/T] | Y |
| 535\|368361 | TGACTTA[C/T]GACATTCCGGGTTTTGGGAGAGGTTATGTGC | | | |
| | ACCTCGAGAGTGGTTATTG | | | |
| Nitab4.5_0000 | TTCGTGTGTTTGTTGATTTGATATTTCCTGAAAATAATTATAT | 470 | [T/C] | Y |
| 535\|368949 | TATAGAC[T/C]TTTTCATCTGCAAATAATTAATTAAATAAGTT | | | |
| | TAAATTGAGATGTTAATA | | | |
| Nitab4.5_0000 | TATAAAACGGGTCAAATATGAATAAGAACCATATTATTCATT | 471 | [G/A] | R |
| 535\|371802 | TAGAAAAT[G/A]GATAACTAATGAGTAAACATGGATAATCA | | | |
| | ATGGGCCTAACATTTACATTT | | | |
| Nitab4.5_0000 | CTGATGTATCAGAATAGGTTGCTTACATTATATCTTTTGGAAT | 472 | [T/C] | Y |
| 535\|372546 | GTGATTT[T/C]ATTTCAAATCCTAAGTAAACATGAGATATTTC | | | |
| | GTGTGCATTGAATTGTTC | | | |
| Nitab4.5_0004 | ATGCTAAATGAATAAGTTCTAATGCATGAAAATAAGTTTCCT | 473 | [C/T] | Y |
| 551\|218036 | AAAGTTTT[C/T]CCCAAAAAGTCAAAAAATGGCCCCAGCCCA | | | |
| | CATGGTTAAAACCCGAGGTT | | | |

TABLE 6
SNPs associated with the Nic3 locus identified in the Examples.

| | | SEQ | | |
|---|---|---|---|---|
| Landmark\|pos | Flanking sequence | ID No. | Variant | iupac |
| Nitab4.5_0001 | TCATGTACTTCACTATTTCTTGCGGATCTATCGTATCGTTGCTT | 474 | [G/T] | K |
| 816\|402186 | CTCTCT[G/T]ATTGTAATTTCCCATGTGCCTTACTATACCGCTA | | | |
| | AGGAAAACAAACAAGA | | | |
| Nitab4.5_0001 | ATTTCTTAATATAAAAAAAGTATTTATTTTCTTGGGACAGACT | 475 | [A/T] | W |
| 816\|402319 | AAAAAAG[A/T]AAGTGTATCACATAAATAGGGACAAATGGA | | | |
| | GTAATAATGTTACTTTATAT | | | |
| Nitab4.5_0001 | TAGAATGCTTAAGTTCAACAATTACAAACAAAAACTTCAGCC | 476 | [G/A] | R |
| 816\|404520 | AATACTAA[G/A]TTTATGCGAAATTTTTTAGCTAAAACATGCT | | | |
| | AAAGTTCARCAAATAGAGA | | | |
| Nitab4.5_0001 | CCAATACTAARTTTATGCGAAATTTTTTTAGCTAAAACATGCTA | 477 | [G/A] | R |
| 816\|404560 | AAGTTCA[G/A]CAAATAGAGAACAAAACTTCAGCTAGTAGA | | | |
| | ATGCTTAAGTTCGGCAATTA | | | |
| Nitab4.5_0001 | CAGATCTGCAGGTGAGAAAATGTCAAATGTAGTAGGCGTAA | 478 | [A/G] | R |
| 816\|406176 | GAGAGTGAG[A/G]CTCTGCCTATAAAAGGAGAGCTTCAGCT | | | |
| | CTCATCTCGACACACTAATCTC | | | |
| Nitab4.5_0001 | CGCTACAACATGAAGAATGATAAGCTAGTCAGCACACCTCTT | 479 | [T/A] | W |
| 816\|409526 | GCTAGTCA[T/A]CTAAAGTTGAGCAAAAAGATGTGTCCTACA | | | |
| | ACAGAAGAGACGAAAGGGAA | | | |
| Nitab4.5_0001 | GTTCCTTCAAGAGCTTGGATTGCATCAGAAGGAGTATATCAT | 480 | [A/G] | R |
| 816\|409791 | CTATTGTG[A/G]CAGTCAAAGTGCAATAGACCTGAGCAAAA | | | |
| | ATACCATGTATCATGCAAGGA | | | |
| Nitab4.5_0001 | CAGATCTGCATGAGAGAAAATGTCAAATGTAGTAGGCGTAA | 481 | [A/G] | R |
| 816\|410234 | GAGAGTGAG[A/G]CTCTGCCTATAAAAGGAGAGCTTCAGCT | | | |
| | CTCATYTCGACACACYAATCTC | | | |
| Nitab4.5_0001 | GGCGTAAGAGAGTGAGRCTCTGCCTATAAAAGGAGAGCTTC | 482 | [T/C] | Y |
| 816\|410268 | AGCTCTCAT[T/C]TCGACACACYAATCTCAGAAGAAAAATATA | | | |
| | TCTGAGAGTTAGAAAGAAAG | | | |
| Nitab4.5_0001 | AGTGAGRCTCTGCCTATAAAAGGAGAGCTTCAGCTCTCATYT | 483 | [C/T] | Y |
| 816\|410278 | CGACACAC[C/T]AATCTCAGAAGAAAAATATATCTGAGAGTT | | | |
| | AGAAAGAAAGAGTGAGGTTT | | | |
| Nitab4.5_0001 | CAGGTTCTGTGTATAGCTCCTTGTAGAATTCAATAATCTCGTT | 484 | [C/A] | M |
| 816\|414572 | CTCTATT[C/A]TTTCTGGATCCTCGATTACTTCATGTCGAATAA | | | |
| | TTATTTGGTCAATGTTG | | | |
| Nitab4.5_0001 | TCATGTACTTCACTATTTCTTGCGGATCTATCGTATCGTTGCTT | 485 | [G/T] | K |
| 816\|402186 | CTCTCT[G/T]ATTGTAATTTCCCATGTGCCTTACTATACCGCTA | | | |
| | AGGAAAACAAACAAGA | | | |
| Nitab4.5_0001 | ATTTCTTAATATAAAAAAAGTATTTATTTTCTTGGGACAGACT | 486 | [A/T] | W |
| 816\|402319 | AAAAAAG[A/T]AAGTGTATCACATAAATAGGGACAAATGGA | | | |
| | GTAATAATGTTACTTTATAT | | | |
| Nitab4.5_0001 | TAGAATGCTTAAGTTCAACAATTACAAACAAAAACTTCAGCC | 487 | [G/A] | R |
| 816\|404520 | AATACTAA[G/A]TTTATGCGAAATTTTTTAGCTAAAACATGCT | | | |
| | AAAGTTCAGCAAATAGAGA | | | |
| Nitab4.5_0001 | ATCTTTGGACCCTTCCGCGAATATTAAAGGCAGCACAATGCA | 488 | [T/C] | Y |
| 816\|405228 | CAAAGTAT[T/C]TTGTATTCACATAGAGTTCGGAAAAAGACC | | | |
| | GCATCAAAAGGAGTGTGATG | | | |
| Nitab4.5_0001 | CAGATCTGCAGGTGAGAAAATGTCAAATGTAGTAGGCGTAA | 489 | [A/G] | R |
| 816\|406176 | GAGAGTGAG[A/G]CTCTGCCTATAAAAGGAGAGCTTCAGCT | | | |
| | CTCATCTCGACACACTAATCTC | | | |
| Nitab4.5_0001 | ACCTACAAAGTGTAAAATTCCTTGCTATAGTGATATCAGTTG | 490 | [A/G] | R |
| 816\|410508 | CTCTTCTC[A/G]GGGCCGTGATTTTTTTTCCCTTATTCAAAAGG | | | |
| | GTTTTCCACGTAAAAATCT | | | |
| Nitab4.5_0001 | AACATTAAATTATTTCTTAATATAAAAAAAGTATTTATTTTCTT | 491 | [G/A] | R |
| 816\|402308 | GGGACA[G/A]ACTAAAAAAGAAAGTGTATCACATAAATAGG | | | |
| | GACAAATGGAGTAATAATG | | | |
| Nitab4.5_0001 | AACTTATACCCTAATTTTTTACAACTTTAGGCCCCTCCTTCTTTC | 492 | [C/A] | M |
| 816\|403841 | TGCTAC[C/A]TGTGCGCTCCTTTCTTCTTCATTTCTTTTCCTCTT | | | |
| | CCGCAAATGAAACTC | | | |
| Nitab4.5_0001 | CCAATACTAAGTTTATGCGAAATTTTTTTAGCTAAAACATGCTA | 493 | [G/A] | R |
| 816\|404560 | AAGTTCA[G/A]CAAATAGAGAACAAAACTTCAGCTAGTAGA | | | |
| | ATGCTTAAGTTCGGCAATTA | | | |
| Nitab4.5_0001 | TGCAAGCATTAATAACTGCTTCTATGGTTCGAACTCGTAACCT | 494 | [G/A] | R |
| 816\|405346 | ATAGACA[G/A]CCTACCCATTCGAGAATATATTCTACAAATAT | | | |
| | TGTACAATATAAACAAAA | | | |
| Nitab4.5_0001 | ATGAGGACGAGGAATGCATGCACCTGTCAGGTCCAAAGTCG | 495 | [G/A] | R |
| 816\|407240 | GAATGGGTG[G/A]TTGACACAGCGGCATCTTACCATGCCACA | | | |
| | CCGGTAAGAGATCCTTTTTTGC | | | |
| Nitab4.5_0001 | TGACTTCTATTCGAACAATTTTGAGCTTAGCAGCTAGCCTAG | 496 | [G/A] | R |
| 816\|409014 | ATCTTGAA[G/A]TGGAGCAGTTGGATGTGAAAACTGCATTTC | | | |
| | TTCATGGAGATTTGGAAGAG | | | |
| Nitab4.5_0001 | TCATTTGCTCCTTGTACTTCCATCTCTTCACTCGGTTGTTTTGA | 497 | [G/A] | R |
| 816\|415808 | TCTGTC[G/A]ACGCTTCTTTAAGTGCAAAGCATATCCATTCCA | | | |
| | TTGCCTTTCTACCCATG | | | |
| Nitab4.5_0001 | CCAAGTATCTTTGTTGGAGTTCCATCTGGTGATGTCAAAAGA | 498 | [C/A] | M |
| 816\|415953 | TTTGAATC[C/A]TGCATTGAAGTATATCCTGTTGTCTCCCATA | | | |
| | TTGTACTAAACAGCTGCTC | | | |
| Nitab4.5_0001 | AATTTTGAAAAGTGGCCGGGATTGAGCTCACGCGCCGGCGC | 499 | [G/T] | K |
| 816\|416247 | GTGGTATTT[G/T]GCTCGCCGGAAAGTGCCGCGCGTGGCGG | | | |
| | CGCGTGAGAGACTGTTTGTCGG | | | |
| Nitab4.5_000 | GGAGAAATACGCAAAAGGAATAAAAGCAACGTCAATTAAAAA | 500 | [T/C] | Y |
| 0556\|800382 | TACATATA[T/C]AAAGAAAATAGGAGAAAGCGAAGAGTACCT | | | |
| | CCTCAAAGAATTAGTTGAAT | | | |
| Nitab4.5_000 | GATGATCTACCACAACATGCCCAAAAGTGCGTCCGAGTACAGT | 501 | [C/G] | S |
| 0556\|802274 | TCTTAAT[C/G]GATATCAGCAATAAAAGCTAAAGGAACTAACG | | | |
| | CCCCAATGACATAAACTC | | | |
| Nitab4.5_000 | TCGCAAATGCGAGACTATGTTCGCATTTGCGAGGTGGGTGAT | 502 | [C/A] | M |
| 0556\|804914 | CGAAATTG[C/A]GACGTATGCTGGGGTCTGTCGGGTTCGCATT | | | |
| | TGCGAACTCAGTGTTCGTA | | | |
| Nitab4.5_000 | GGACTCGAGATTAATTTTTTGGGGAATTATGTAAAGATCATAGC | 503 | [A/G] | R |
| 0556\|805520 | TTTATTT[A/G]TTGGAATTAATTTCTCTTGCATTATTTGATGTTA | | | |
| | CTAAGCTGATTTTGGT | | | |
| Nitab4.5_000 | TTTGCACTATTTGAACTACGTGAAAGGCGTGTACTCGAGGTGA | 504 | [A/G] | R |
| 0556\|805755 | CGAGTGT[A/G]TACACGGCCTTATATATGAAAATTTGACTGGT | | | |
| | TTAGACTCTTAGGTTTCT | | | |
| Nitab4.5_000 | GTTGTTATATTATATTGTTACGCTTTTGGTACATGTGGGATTGT | 505 | [G/A] | R |
| 0556\|806405 | TGAGAG[G/A]ATTGTCGGGGTATTTGCACGTGAGTTGTCTGT | | | |
| | GCTAAAGTTATTATTAAT | | | |
| Nitab4.5_000 | AGTTTTGGCCCAAAAATAAAATAATTTTTTTTCATTAGCTTATAG | 506 | [C/T] | Y |
| 0556\|808699 | TAACTT[C/T]ACAACTCCACTTCCTTCAACAACTCATGTGTCTA | | | |
| | GGCCCAAACATTGGAT | | | |
| Nitab4.5_000 | TATAAATGAACACCAAAGAACACCAAAAGAAAAAGAAGCAAC | 507 | [T/C] | Y |
| 0556\|809145 | CCATATCA[T/C]TAATCAAAACAAGATAAGAAGTGGACCTCAA | | | |
| | AAGTTGAATCTGAAAATTT | | | |
| Nitab4.5_000 | AATTTTTTATTATTAAACACAATTTATGTCGAGGTCGTACGACCC | 508 | [G/A] | R |
| 0556\|820786 | GATCCA[G/A]AATAATGTGTACACTACCAATGATCGAGCGGCA | | | |
| | CGAACCATAGATGCATC | | | |
| Nitab4.5_000 | GCCGAGGCATTCGGCCTACTCCACAAGAAGAGAAGGATATAT | 509 | [T/C] | Y |
| 0556\|820895 | TATAAGCA[T/C]TTGACTTAAACATTTTTTGAGGTTTTATACAAA | | | |
| | AGCAATACGAATTTCATT | | | |
| Nitab4.5_000 | ATCCCATTCCATAACTATTTTCGTGGTTAAAATCTCATTTTTTATC | 510 | [T/C] | Y |
| 0556\|821499 | AAAAA[T/C]CTAGGTTTTTCATCTTAACCCTTGATTTCCACAGTT | | | |
| | TACATGTTATAATC | | | |
| Nitab4.5_000 | GCGGCTGTCCATGCAGAGGTGCGGTTACACTAGAAGCTAGAA | 511 | [C/T] | Y |
| 0556\|822015 | GCTTCAAC[C/T]ATTGCTCCTAAGTCCAAACTTGGTCCGAGCCT | | | |
| | CGTCCGATTAACACCCGA | | | |
| Nitab4.5_000 | TGATTGGTAATGTGGGCACGAGATGCCAAGTGATTATAATTC | 512 | [G/A] | R |
| 0556\|822766 | GTGATTTG[G/A]GACTTGTGATTGTGATTATGAGGTGTGGTAC | | | |
| | CTCAGGGTGATTCTCGTGT | | | |
| Nitab4.5_000 | GTACAAGTTCTTATGTCTAGTAGGTGCCTCGACTGTACCTCGT | 513 | [C/T] | Y |
| 0556\|823091 | CACTACT[C/T]CACCGAAGTTAGTCTTGATACTTACTGGGTACC | | | |
| | GTTGTGGTGTACTCATG | | | |
| Nitab4.5_000 | TAGGCTTACCTAGTCTTAGAGATTAAGTGTAATCGCGACATCT | 514 | [G/A] | R |
| 0556\|823531 | TAAGGTA[G/A]AAATTTGGGATCGTGATAAGTTGGTATGTTGA | | | |
| | GAATGAACCCTTTCTATA | | | |
| Nitab4.5_000 | CGTCCACAAAACAATGTGAACTTAGGGAATCAATGTAATATTT | 515 | [T/C] | Y |
| 0556\|823836 | TTTTTGG[T/C]CATTGGGAATTCAATGTAATATATATGCATGCA | | | |
| | TCCACATATATAATTTG | | | |
| Nitab4.5_000 | CGAAGCTAATTTATTTATTATTTTTTATTTTTTATTTTAAAATCATT | 516 | [G/T] | K |
| 0556\|803248 | AGGTG[G/T]GGATTGGGGAAGTAAGATGATAATGTCTATATT | | | |
| | ATTCGAACTATTTTATT | | | |
| Nitab4.5_000 | GGACTCGAGATTAATTTTTTGGGGAATTATGTAAAGATCATAGC | 517 | [A/G] | R |
| 0556\|805520 | TTTATTT[A/G]TTGGAATTAATTTCTCTTGCATTATTTGATGTTA | | | |
| | CTAAGCTGATTTTGGT | | | |
| Nitab4.5_000 | TTTGCACTATTTGAACTACGTGAAAGGCGTGTACTCGAGGTGA | 518 | [A/G] | R |
| 0556\|805755 | CGAGTGT[A/G]TACACGGCCTTATATATGAAAATTTGACTGGT | | | |
| | TTAGACTCTTAGGTTTCT | | | |
| Nitab4.5_000 | NNNNNNNNNNNNNNNNNNNNNNNNNNNNNNNNNNGAG | 519 | [T/G] | K |
| 0556\|819811 | AGAGAGAGAGAGA[T/G]AGATATGCTCATCCAAAGTTTGATC | | | |
| | TTATGCGTACTCATTTAGAACTTTA | | | |
| Nitab4.5_000 | ATCCCATTCCATAACTATTTTCGTGGTTAAAATCTCATTTTTTATC | 520 | [T/C] | Y |
| 0556\|821499 | AAAAA[T/C]CTAGGTTTTTCATCTTAACCCTTGATTTCCACAGTT | | | |
| | TACATGTTATAATC | | | |
| Nitab4.5_000 | AAACGAGACAAATTTGCGTAGTATGGAGCTGGAAAAATTAAA | 521 | [C/T] | Y |
| 0556\|797881 | TTTGACGC[C/T]GATTCATATATATTACCCTAACAAATTTGAGA | | | |
| | TTTGGTCTAGTTGATTGA | | | |
| Nitab4.5_000 | TAAACTGGAACAAAGGAAGTATCAACTTTTTTGAGTCTGAGGA | 522 | [C/T] | Y |
| 0556\|798666 | AAAGTGGG[C/T]TGTAGGAAGCGCTCGCTTCTTAGGCAGAGC | | | |
| | ATGAAGCGATGAAGATGCGA | | | |
| Nitab4.5_000 | CGCTTTCAAAATCACTGGTGACAAGACAGCATTTCAGGGCTCT | 523 | [C/T] | Y |
| 0556\|799620 | ACAATAC[C/T]TTTACATACACTCATCTAACAACCTTGCAGCTT | | | |
| | AGGTTGTAGCAATGCTG | | | |
| Nitab4.5_000 | GGAGAAATACGCAAAAGGAATAAAAGCAACGTCAATTAAAAA | 524 | [T/C] | Y |
| 0556\|800382 | TACATATA[T/C]AAAGAAAATAGGAGAAAGCGAAGAGTACCT | | | |
| | CCTCAAAGAATTAGTTGAAT | | | |
| Nitab4.5_000 | GATGATCTACCACAACATGCCCAAAAGTGCGTCCGAGTACAGT | 525 | [C/G] | S |
| 0556\|802274 | TCTTAAT[C/G]GATATCAGCAATAAAAGCTAAAGGAACTAACG | | | |
| | CCCCAATGACATAAACTC | | | |
| Nitab4.5_000 | ATGTCGTACACTTGTCGTTTGGTACACATTGCAAATCGCGCCA | 526 | [C/T] | Y |
| 0556\|808321 | TGTGAAA[C/T]GCACGCGCCACGTGAAACGCACGCGCGATTT | | | |
| | ACAACATGTTTATTATTAT | | | |
| Nitab4.5_000 | ACACTTATTATCATATCCAATTAATATCCATCATATTAATAGTCC | 527 | [T/C] | Y |
| 0556\|819988 | TCGTC[T/C]ACATACAAAATAATATAATTAGCGCATATCAACTT | | | |
| | TCTTAATAGTGCTTA | | | |
| Nitab4.5_000 | AATTTTTTATTATTAAACACAATTTATGTCGAGGTCGTACGACCC | 528 | [G/A] | R |
| 0556\|820786 | GATCCA[G/A]AATAATGTGTACACTACCAATGATCGAGCGGCA | | | |
| | CGAACCATAGATGCATC | | | |
| Nitab4.5_000 | TTGCGGTTGTGATTGGTAATGTGGGCACGAGATGCCAAGTGA | 529 | [C/T] | Y |
| 0556\|822757 | TTATAATT[C/T]GTGATTTGRGACTTGTGATTGTGATTATGAGG | | | |
| | TGTGGTACCTCAGGGTGA | | | |
| Nitab4.5_000 | TGATTGGTAATGTGGGCACGAGATGCCAAGTGATTATAATTY | 530 | [G/A] | R |
| 0556\|822766 | GTGATTTG[G/A]GACTTGTGATTGTGATTATGAGGTGTGGTAC | | | |
| | CTCAGGGTGATTCTCGTGT | | | |
| Nitab4.5_000 | GTACAAGTTCTTATGTCTAGTAGGTGCCTCGACTGTACCTCGT | 531 | [C/T] | Y |
| 0556\|823091 | CACTACT[C/T]CACCGAAGTTAGTCTTGATACTTACTGGGTACC | | | |
| | GTTGTGGTGTACTCATG | | | |
| Nitab4.5_000 | CCTAGTCTTAGAGATTAAGTGTAATCGCGACATCTTAAGGTAG | 532 | [G/A] | R |
| 0556\|823539 | AAATTTG[G/A]GATCGTGATAAGTTGGTATGTTGAGAATGAAC | | | |
| | CCTTTCTATACTCTGTAT | | | |
| Nitab4.5_000 | CGTCCACAAAACAATGTGAACTTAGGGAATCAATGTAATATTT | 533 | [T/C] | Y |
| 0556\|823836 | TTTTTGG[T/C]CATTGGGAATTCAATGTAATATATATGCATGCA | | | |
| | TCCACATATATAATTTG | | | |

TABLE 7
SNPs associated with marker Nt2AG2015.

| | | SEQ | | |
|---|---|---|---|---|
| Landmark\|pos | Flanking sequence | ID No. | Variant | iupac |
| Nitab4.5_0002 | GACCGTTATATTATACTGGAATTCCAGTATATATTATTGAAG | 534 | [C/T] | Y |
| 539\|87422 | TATTTTTTC[C/T]GGATTTTGTATAGTGTTTTCGTTCAGATTTAT | | | |
| | CTTTACATGAAAAGTGGC | | | |
| Nitab4.5_0002 | AGCGCTGTCTTCTTACACATTAATTGTATTGTGTGTTTATTAC | 535 | [G/A] | R |
| 539\|89149 | TCCCTCC[G/A]TCTCATAGTACCTGTCATGATTCTCTTGTGCA | | | |
| | CGCCCCTTAAGAAAAATT | | | |
| Nitab4.5_0002 | AAACATGATAAAGTGTTTAAGAAAACTAGGTTGACTCATTG | 536 | [C/A] | M |
| 539\|89367 | GTATTTGAA[C/A]AAAGTTATTCATAATTGAGGTGTTTGTAG | | | |
| | TCTTTAAGGATAATTATTACC | | | |
| Nitab4.5_0002 | ACGTGGTTCTTATCCACACAAAAATGAATTTTCCCATTCGGT | 537 | [C/T] | Y |
| 539\|90547 | ATTCAATA[C/T]CGRTATTGAGACCCGATTAAATTTAGATTCA | | | |
| | TGCTAGAAAAGTCCCCCGT | | | |
| Nitab4.5_0002 | TGGTTCTTATCCACACAAAAATGAATTTTCCCATTCGGTATTC | 538 | [G/A] | R |
| 539\|90550 | AATAYCG[G/A]TATTGAGACCCGATTAAATTTAGATTCATGC | | | |
| | TAGAAAAGTCCCCCGTTGA | | | |
| Nitab4.5_0002 | CCCGATTAAATTTAGATTCATGCTAGAAAAGTCCCCCGTTGA | 539 | [A/G] | R |
| 539\|90609 | GAGGTAAA[A/G]TGGTCTCTCATAAAGACGAATTTATACTTA | | | |
| | GGGCTCGAATCCGAGACTTC | | | |
| Nitab4.5_0002 | CGCGAAATCCACAACAGACGATTGCCAAAATATGGCATACG | 540 | [C/T] | Y |
| 539\|94658 | TCCACCCTT[C/T]GCGAGCCCCATCGATTAAAGTCTGGAGTC | | | |
| | GCTGTTGCAGTGACTCTTGGT | | | |
| Nitab4.5_0002 | CCCTTTGTGGTTTGCAGGTTATTACACAGGGAAAGTTTACCC | 541 | [C/T] | Y |
| 539\|96112 | AGTACACA[C/T]AAAGTGCTCACCACAGAGTGCTCATCCGAA | | | |
| | GGGTAGAGGCTGTAGCAAAG | | | |
| Nitab4.5_0002 | TCTAATTTTCAAATATTATTTTCACTTAAAAAAAGATTTCACTT | 542 | [G/A] | R |
| 539\|99931 | TTTTTG[G/A]AATTTTACAATTCTTATGTCCAAGCGCCAACTA | | | |
| | TTTTTGTTCATTTAAAT | | | |
| Nitab4.5_0002 | ACTCATAAACATGTTTCACACCTTACACTACTAGAAGTTCGAT | 543 | [G/C] | S |
| 539\|101581 | AAAAATC[G/C]ACCGGAGCTAACCGGTCGATATTGTAATTTA | | | |
| | CATATGGATTCATTTSAAT | | | |
| Nitab4.5_0002 | ATCSACCGGAGCTAACCGGTCGATATTGTAATTTACATATGG | 544 | [G/C] | S |
| 539\|101628 | ATTCATTT[G/C]AATTTTGAACTAAAATCTATCAAAATTAGTG | | | |
| | AAAATATTGAGCTTTGAAC | | | |
| Nitab4.5_0002 | CCAACAACTGTCAGGCAGCTAACAGCTTGTTTGGATGGTTGT | 545 | [G/A] | R |
| 539\|104045 | TACTCATT[G/A]TATCGTATTGTATTGTATTGTATTGTTATCTC | | | |
| | AAAATAATGCTTGTTTTG | | | |
| Nitab4.5_0002 | CTAGACAACGTGCTCTTAGGCTGATGCTTTCTGAATTGTTTC | 546 | [A/G] | R |
| 539\|108145 | CAAGGAAT[A/G]CTTCTATCATATGTTCTGCCTTTCTTCCTCTT | | | |
| | TTTTCCTTTGCTTCTCCT | | | |
| Nitab4.5_0002 | AAATTTCAGCACACTTGGTTCAACACACTTGCTACTTCAGTCC | 547 | [T/C] | Y |
| 539\|108639 | CGTCTAA[T/C]AGAATGCTGAAGTTTTGTGTGATTGTCTTTGC | | | |
| | TACTTCAGCCCTGTATGC | | | |
| Nitab4.5_0002 | CCTGTATGCTGAAGTTACGCGAAAAAGTAGGTACGCTTGCA | 548 | [G/A] | R |
| 539\|108731 | ATTTTTTTT[G/A]CAAAGCGGGCACAAGTTAAAACGTGACTC | | | |
| | AAAAAGCGGATATAGATGCAA | | | |
| Nitab4.5_0002 | CATCATCTATGAGTGGGGTCCAGGGACGGACCATAAAAATC | 549 | [C/T] | Y |
| 539\|109433 | TACTGTATT[C/T]AATTTTATACTRTATTTCTGCAAAAGATTAT | | | |
| | TTTCACGGCAACAACCCGT | | | |
| Nitab4.5_0002 | GTGGGGTCCAGGGACGGACCATAAAAATCTACTGTATTYAA | 550 | [G/A] | R |
| 539\|109445 | TTTTATACT[G/A]TATTTCTGCAAAAGATTATTTTCACGGCAA | | | |
| | CAACCCGTGACCTCTTGGTC | | | |
| Nitab4.5_0002 | AAACATGATAAAGTGTTTAAGAAAACTAGGTTGACTCATTG | 551 | [C/A] | M |
| 539\|89367 | GTATTTGAA[C/A]AAAGTTATTCATAATTGAGGTGTTTGTAG | | | |
| | TCTTTAAGGATAATTATTACC | | | |
| Nitab4.5_0002 | TGGTTCTTATCCACACAAAAATGAATTTTCCCATTCGGTATTC | 552 | [G/A] | R |
| 539\|90550 | AATACCG[G/A]TATTGAGACCCGATTAAATTTAGATTCATGC | | | |
| | TAGAAAAGTCCCCCGTTGA | | | |
| Nitab4.5_0002 | CCCGATTAAATTTAGATTCATGCTAGAAAAGTCCCCCGTTGA | 553 | [A/G] | R |
| 539\|90609 | GAGGTAAA[A/G]TGGTCTCTCATAAAGACGAATTTATACTTA | | | |
| | GGGCTCGAATCCGAGACTTC | | | |
| Nitab4.5_0002 | GTATATAAAATTTGTATATTTTTTTTTGTATGTAACATATATAT | 554 | [A/T] | W |
| 539\|91027 | ATATATA[A/T]TAAAAATATAATTGTTTGCTATTATTTTGAGA | | | |
| | GCGGCTATACGGTGTCCT | | | |
| Nitab4.5_0002 | CGCGAAATCCACAACAGACGATTGCCAAAATATGGCATACG | 555 | [C/T] | Y |
| 539\|94658 | TCCACCCTT[C/T]GCGAGCCCCATCGATTAAAGTCTGGAGTC | | | |
| | GCTGTTGCAGTGACTCTTGGT | | | |
| Nitab4.5_0002 | GATCGATCGGATTTAGCATATTTCGGATTTCGGATTATAGAA | 556 | [T/C] | Y |
| 539\|95344 | AATGCAAT[T/C]CGAATTCGATCCTAAATATATCGGATCGGA | | | |
| | TCGGATTTTAAAGTTTGGAT | | | |
| Nitab4.5_0002 | ATCGACCGGAGCTAACCGGTCGATATTGTAATTTACATATGG | 557 | [G/C] | S |
| 539\|101628 | ATTCATTT[G/C]AATTTTGAACTAAAATCTATCAAAATTAGTG | | | |
| | AAAATATTGAGCTTTGAAC | | | |
| Nitab4.5_0002 | CCAACAACTGTCAGGCAGCTAACAGCTTGTTTGGATGGTTGT | 558 | [G/A] | R |
| 539\|104045 | TACTCATT[G/A]TATCGTATTGTATTGTATTGTATTGTTATCTC | | | |
| | AAAATAATGCTTGTTTTG | | | |
| Nitab4.5_0002 | CCTGTATGCTGAAGTTACGCGAAAAAGTAGGTACGCTTGCA | 559 | [G/A] | R |
| 539\|108731 | ATTTTTTTT[G/A]CAAAGCGGGCACAAGTTAAAACGTGACTC | | | |
| | AAAAAGCGGATATAGATGCAA | | | |
| Nitab4.5_0002 | GTGTTTTATTTTTTATCCATTTGTCTAAGCTTTTTTGGTAGACAT | 560 | [A/G] | R |
| 539\|87891 | AGTTACT[A/G]GATACCTGTGCGGGTTGAAAATAGCAGGTA | | | |
| | TTTGGTAAAATTAATTGAAG | | | |
| Nitab4.5_0002 | GTGTTATTTCATATGTACTAAATATTTTTTTAGGAGATTGAGTT | 561 | [A/G] | R |
| 539\|88735 | GAGGGTA[A/G]GGGTTGTTTTTTTTGGATTTTTTGTTTTCTCCC | | | |
| | ATTTTATTGGAGATGTTA | | | |
| Nitab4.5_0002 | ATATAAAATTTGTATATTTTTTTTTGTATGTAACATATATATAT | 562 | [A/T] | W |
| 539\|91029 | ATATAAT[A/T]AAAATATAATTRTTTGCTATTATTTTGAGAGC | | | |
| | GGCTATACGGTGTCCTTT | | | |
| Nitab4.5_0002 | TATATTTTTTTTTGTATGTAACATATATATATATATAATWAAAA | 563 | [G/A] | R |
| 539\|91041 | TATAATT[G/A]TTTGCTATTATTTTGAGAGCGGCTATACGGT | | | |
| | GTCCTTTTCCTTTAAAATT | | | |
| Nitab4.5_0002 | TGCACGGATCAGATCGATCGGATTTAGCATATTTCGGATTTC | 564 | [G/A] | R |
| 539\|95333 | GGATTATA[G/A]AAAATGCAATTCGAATTCGATCCTAAATAT | | | |
| | ATCGGATCGGATCGGATTTT | | | |
| Nitab4.5_0002 | CTTTGTAAAAGAGACAATACATTAAGAAAAATTCATGTTTAT | 565 | [G/A] | R |
| 539\|95503 | GCAATTAT[G/A]AGAGTACTATGGTGCCAATATAGCTAAATC | | | |
| | TAGCAATTGTAAAGGTAATA | | | |
| Nitab4.5_0002 | CAGACCGCCTAAGAGACTTTCCGACTGTCGAAGGAAGGAAG | 566 | [T/C] | Y |
| 539\|98944 | TTCAGTTTT[T/C]AGTGACCAGAAATTGACTAGTTTATTTAAT | | | |
| | TGACTTAAAGATCTTTCTTT | | | |
| Nitab4.5_0002 | AAAATTTGATACGAACTAAAAAGGAAATTGGTTCACTTAAAT | 567 | [G/T] | K |
| 539\|105559 | AAAATGAA[G/T]AGTTATTTTTTATTTGACCAGAATTGACGCG | | | |
| | GCCCTGCAACTGAACACGCG | | | |
| Nitab4.5_0002 | TCTAGAGCTAAAGTTCGTCAGTTACAAAAACAAAAACTTCAG | 568 | [A/G] | R |
| 539\|108521 | CAATTACA[A/G]ACAAAAATTTCAGCAAATAGAGAACAAAA | | | |
| | CTTCAGCTACTATGCTTAAGT | | | |
| Nitab4.5_0002 | GTGGGGTCCAGGGACGGACCATAAAAATCTACTGTATTCAA | 569 | [G/A] | R |
| 539\|109445 | TTTTATACT[G/A]TATTTCTGCAAAAGATTATTTTCACGGCAA | | | |
| | CAACCCGTGACCTCTTGGTC | | | |

In one embodiment provided herein are markers for use in identifying plants with low levels of nicotine.

SNPs or markers for use in genotyping the Nic1 and/or Nic2 locus are available in WO2018237107 which is incorporated herein by reference.

As used herein, “SNP” or “single nucleotide polymorphism” means a sequence variation that occurs when a single nucleotide (A, T, C or G) in the genome sequence is altered or variable with respect to a reference sequence. “SNP markers” exist when SNPs are mapped to sites on the genome.

As used herein, “marker” or “SNP marker” means a nucleic or amino acid sequence which is sufficiently unique to characterise a specific locus on the genome. A polymorphic trait can be used as a marker if it is inherited differentially and exhibits linkage disequilibrium with a phenotypic trait of interest. When a trait is stated to be linked to a given marker it will be understood that the actual DNA segment whose sequence affects the trait usually co-segregates with the marker.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc. other than those specifically described herein. In addition, this disclosure may include other inventions not presently claimed, but which may be claimed in future.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” or “a nitrate reductase” includes a plurality of such candidate agents and equivalents thereof known to those skilled in the art, and so forth.

### Advantages

It has been surprisingly found that by modulating the activity or expression of a Nic3 gene as taught herein, for example by providing a mutation in a Nic3 locus, the alkaloid content (e.g. nicotine content) and/or TSNA precursor content of tobacco cells and tobacco plants or parts thereof can be modulated. Thereby delivery systems with modulated alkaloid (e.g. reduced nicotine) and/or reduced TSNA precursor content and commercially desirable traits sought after by consumers of delivery systems can be produced. In particular, tobacco cells and tobacco plants or parts thereof having reduced nicotine content may be produced by providing at least one mutation in a Nic3 locus, and optionally at least one mutation in a Nic1 locus and/or at least one mutation in a Nic2 locus.

The present inventors have for the first time identified a new locus which is capable of producing an ultra low nicotine phenotype. Prior to the present invention, it had not been known that modulation of the activity or expression of a Nic3 gene as described herein could be used to modulate alkaloid and/or TSNA content.

The present inventors have determined that the modulation of a new locus, referred to herein as the Nic3 locus can reduce the alkaloid content (e.g. nicotine content) of the modified plant to a surprisingly low level. In particular the present inventors have determined that the alkaloid content (e.g. nicotine content) may be reduced to a surprisingly low level by providing at least one mutation in a Nic3 locus, and optionally at least one mutation in a Nic1 locus and/or at least one mutation in a Nic2 locus.

The publications discussed herein are provided solely for their disclosure prior to the filing date of the present application. Nothing herein is to be construed as an admission that such publications constitute prior art to the claims appended hereto.

### EXAMPLES

### Example 1—Development of a Population Segregating for Nic3

A flue-cured tobacco variety (FC101) containing nic1 and nic2, was found to have lower than expected nicotine levels, based on these two loci alone.

It was hypothesized by the present inventors that a third locus, Nic3, was controlling the reduced nicotine levels in this variety. Here we present the work on identifying the underlying gene(s) controlling this locus.

Plant Materials

In order to develop a population segregating for Nic3, an F₂ population of 262 individuals was generated from a cross between FC101 (nic1 nic2 nic3) and LAFC53 (nic1 nic2 Nic3).

All individuals, along with five replicates of the two parents were seeded in the greenhouse and then transplanted into field in Kernersville, N.C. during the normal USA growing season. Plants were grown until 140 days post-transplant before harvesting. Lower, middle and upper stalk leaf positions were harvested as they became mature. Upper leaves (the top 5-7 leaves) were then cured in a six-rack flue curing barn as per standard flue-curing practices.

### Example 2—Phenotyping of Parents and F₂s Derived from the Cross Between FC101 (Nic1 Nic2 Nic3) and LAFC53 (Nic1 Nic2 Nic3)

Nicotine and nornicotine were measured in three technical replicates for both of the parents, as well as 218 individuals from the F2 population.

Alkaloids we measured via standard gas chromatographic methods.

Results

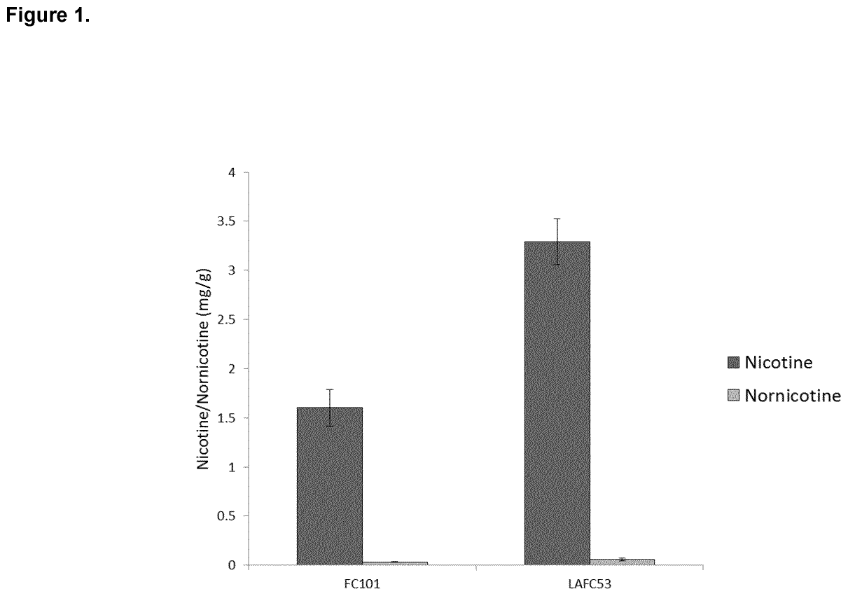

Nicotine and nornicotine content analysis of the two parents showed that FC101 contained significantly lower levels of both alkaloids compared to LAFC53 (FIG. 1).

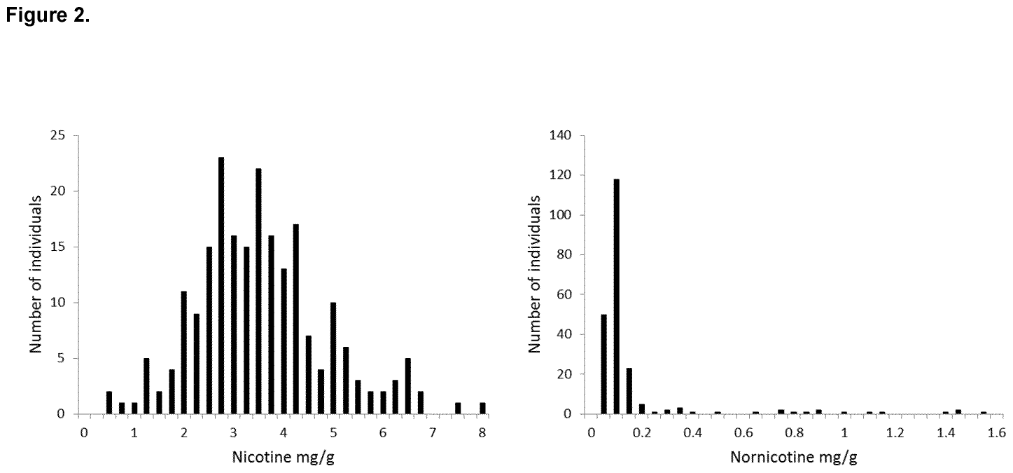

Nicotine levels for the F₂ plants derived from FC101×LAFC53 were found to be continuously distributed (FIG. 2A), so the genotype of Nic3 could not be unambiguously inferred based on the phenotype value.

Nornicotine content in the F₂s was found to be largely uniform, with a few individuals exhibiting spontaneously high levels of nornicotine (FIG. 2B).

### Example 3—Marker Development and Linkage Analysis

DNA was extracted from leaf samples of all F₂ lines and their respective parents using the CTAB method. All F₂ lines and the parental DNA samples were selected for SNP genotyping with a custom tobacco 50K Infinium iSelect HD BeadChip (Illumina Inc., San Diego, Calif.). SNP clusters were generated using GenomeStudio version 2.0 (IIlumina Inc., San Diego, Calif.) and all polymorphic markers identified were used for further analysis. A genetic linkage map for the population was constructed using the software Joinmap version 4.0 (Stam, 1993) using the regression mapping function, with default settings.

In order to rough map the nic3 locus, multiple QTL mapping was carried out on the two populations using the stepwiseqtI function of R/QTL (Broman & Sen, 2009; Manichaikul et al. 2009), using the Haley-Knott regression method, with genotype probabilities calculated at a maximum distance of 1 cM and 1000 permutations to determine logarithm of the odds (LOD) significant thresholds for incorporating both additive QTL and epistatic interactions at an experiment-wise α=0.01.

Results

iSelect HD BeadChip Genotyping of the F₂ Individuals Derived from FC101×LAFC53

To identify the Nic3 locus via QTL analysis, we then genotyped the F₂ individuals with a custom 50K Infinium iSelect HD BeadChip. Using the iSelect HD BeadChip, we identified approximately 4,400 SNP markers that were polymorphic between FC101 and LAFC53.

These markers were able to be mapped to 2992 unique loci in the F2 population.

QTL analysis of the F₂ population identified a linkage group containing a number of markers that was significantly associated with total nicotine content (maximum LOD score of 22.17), explaining 37.4% of the variance in this trait (FIG. 3).

These markers co-located with markers on linkage group 5 of the N. tabacum 30k Infinium HD consensus map 2015 (pseudo-chromosome 5 of the Edwards et al. (2017) genome).

Results

Using the markers identified as being closely linked to the peak of the QTL for nicotine content, we identified a genomic region encompassing the Nic3 locus subtended by the markers Nt1AG1750 (SEQ ID No. 311) and Nt1AC2307 (SEQ ID No. 312) (206 cM to 398 cM in FIG. 3).

Utilizing the BioNano hybrid assembly of Edwards et al. (2017), we were able to identify scaffolds mapped to the pseudo-chromosomes that covered a large part of this region. Markers that were not able to be placed on the pseudo-chromosomes, but were able to be uniquely mapped to a scaffold in the Edwards et al. (2017) genome, were integrated based on their position on the genetic map. We then utilized RNA-seq information from Edwards et al. (2017) in order to improve the gene models for genes within this identified region.

Candidate genes were then chosen based on predicted functions.

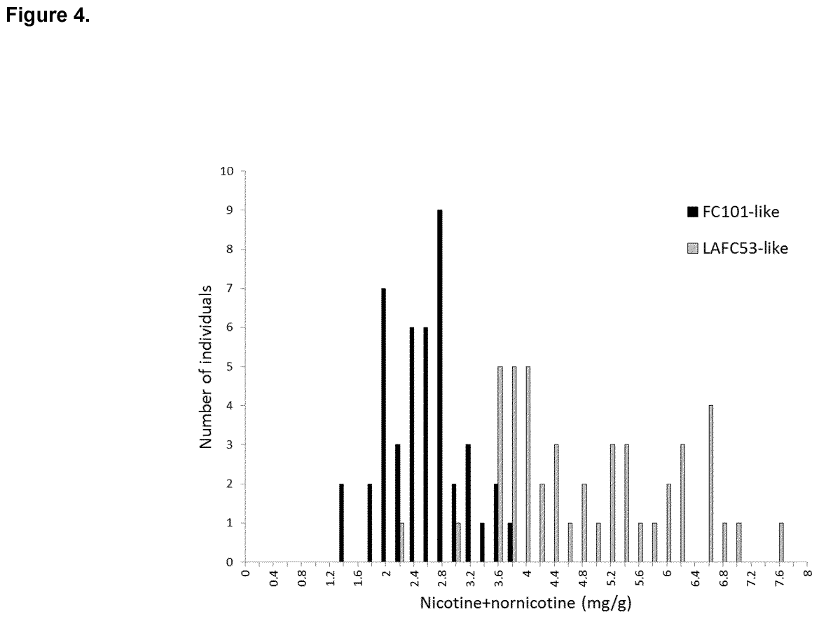

A MYC transcription factor (Nitab4.5_0002539 g0040.1) was identified that contained SNPs in its coding region (marker ID Nt2AG2015) that resulted in an amino acid change (K87E, wherein K is the wild-type) and G84V. F₂ individuals classified as containing FC101 or LAFC53 alleles at this marker exhibited a clear segregation for the nicotine and nornicotine content (FIG. 4), indicating that this alteration may be causal for the low nicotine phenotype.

### Example 4—Physical Mapping and Candidate Gene Identification

SNP markers found to be closely genetically linked to the Nic3 locus were identified by using the Iodint function of R/QTL (Broman & Sen, 2009), using a LOD drop of 1.5 to define the region. Markers within the region of interest surrounding the Nic3 locus that were able to be uniquely anchored to an improved tobacco genome assembly (Edwards et al., 2017) were used to identify BioNano hybrid scaffolds (i.e. pseudo-chromosome regions) subtending the region and therefore identify the chromosome on which Nic3 resides. Gaps in pseudo-chromosome sequences were filled by markers that were able to be uniquely mapped to the genome scaffolds, but were not present on the BioNano hybrid scaffold, based on their relative locations in the genetic map.

Gene model candidates in the updated region were then compared against RNA-seq data (Edwards et al., 2017) and amended if necessary.

### Example 5—Identification of Candidate Gene

To identify the gene(s) within the Nic3 locus responsible for the nicotine modulation observed in FC101, each gene is silenced individually in a low nicotine background (i.e. nic1nic2 background) by virus-induced gene silencing (VIGS), for example as described in WO2020/025963 and the alkaloid content is measured.

### Example 6—Modulation of Activity of Candidate Gene

To confirm that amino acids of interest are required for the protein function, two approaches are used:
1. Gene editing to mutate the residues of interest (e.g. G84V and/or K87E for MYC2) in a low nicotine background (i.e. nic1nic2 background)
2. Overexpression of the unmutated gene as well as gene edited variants (e.g. full-length MYC2, MYC2 G84V, MYC2 K87E and MYC2 G84V K87E)

Functional Domain:

In order to correlate the ultra-low nicotine phenotype with the function of our gene of interest, two approaches are used:
1. Gene editing to delete the functional domain (e.g. DNA-binding site for MYC2)
2. Overexpression of the full-length protein as well as the version comprising a deletion in the functional domain (e.g. full-length MYC2 and MYC2deltaDNA-binding domain) Alkaloid content is measured.

### Example 7—Virus-Induced Gene Silencing of Genes in the Nic3 Locus

A TRV vector comprising both (TRV RNA1, SEQ ID No. 570) and (TRV RNA2, selected from SEQ ID Nos. 571-574) comprising the targeted nucleotide sequence (from SEQ ID NOs: 73 (Nitab4.5_0002539 g0040.2), 118 (Nitab4.5_0002683 g0080.2), 124 (Nitab4.5_0005412 g0010.2) and 127 (Nitab4.5_0005412 g0020.2) were separately propagated in A. tumefaciens. These cultures were mixed (1:1) and syringe-infiltration into 2-week-old LaBY21 plants which have a nic1nic2 background (carrying ERF199 and ERF189 mutations in Nic1 and Nic2 genes, respectively, as disclosed in WO2018/237107). The silencing effect was assessed five weeks post-virus infection by assessing the expression level of the target gene (data not shown).

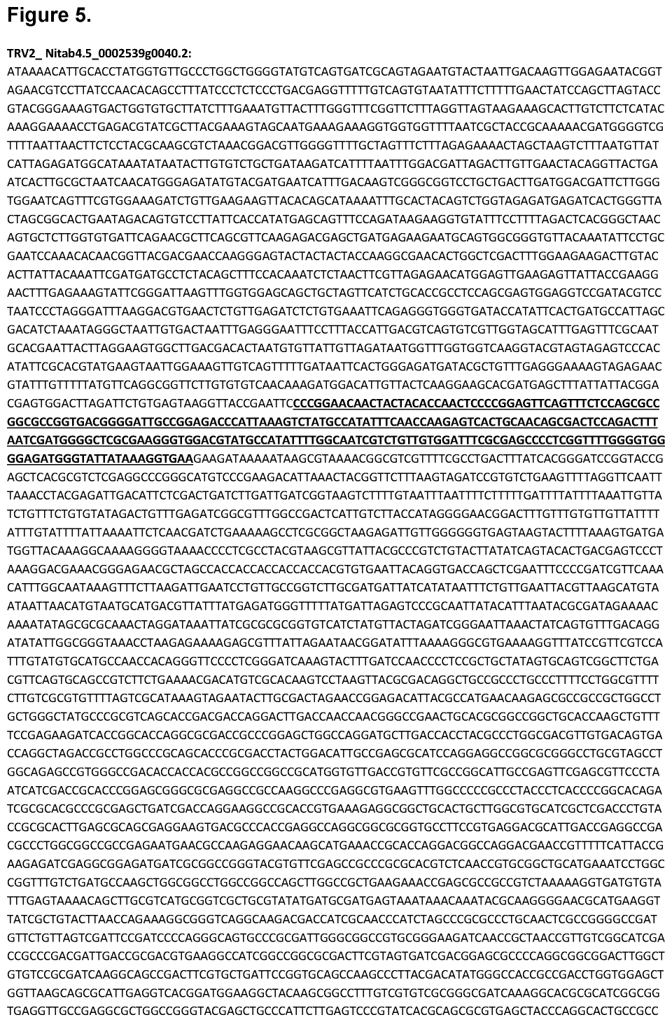

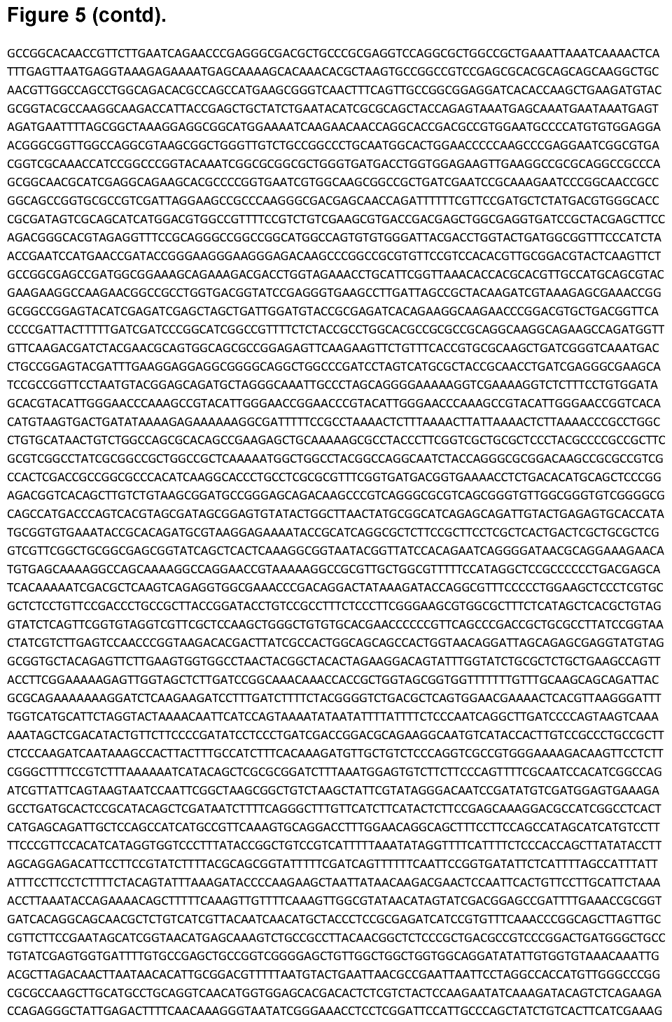

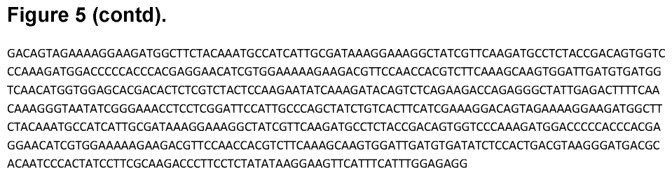

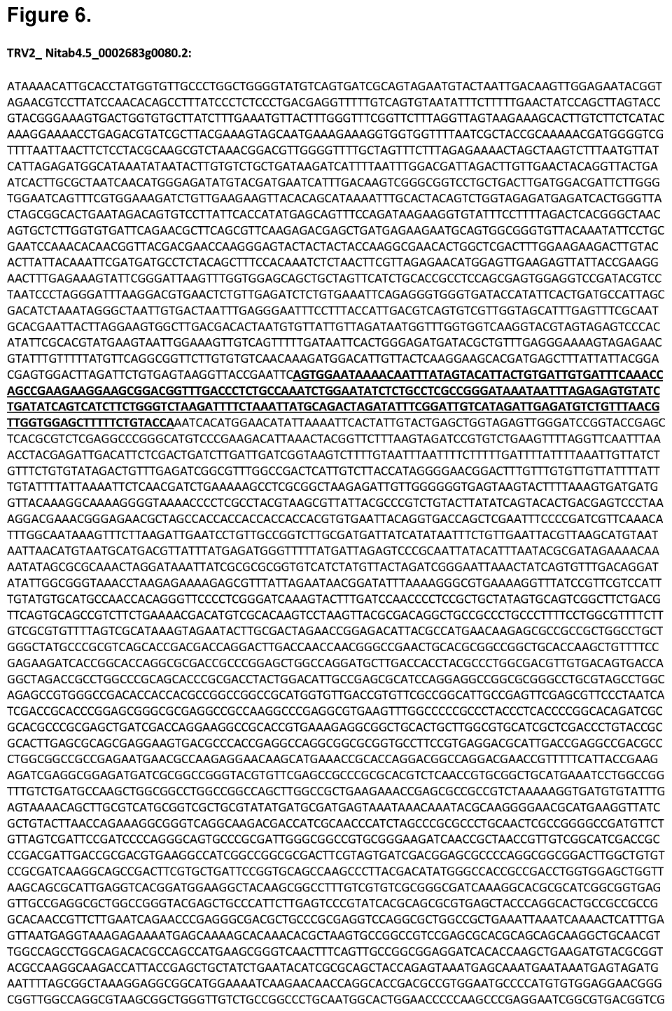

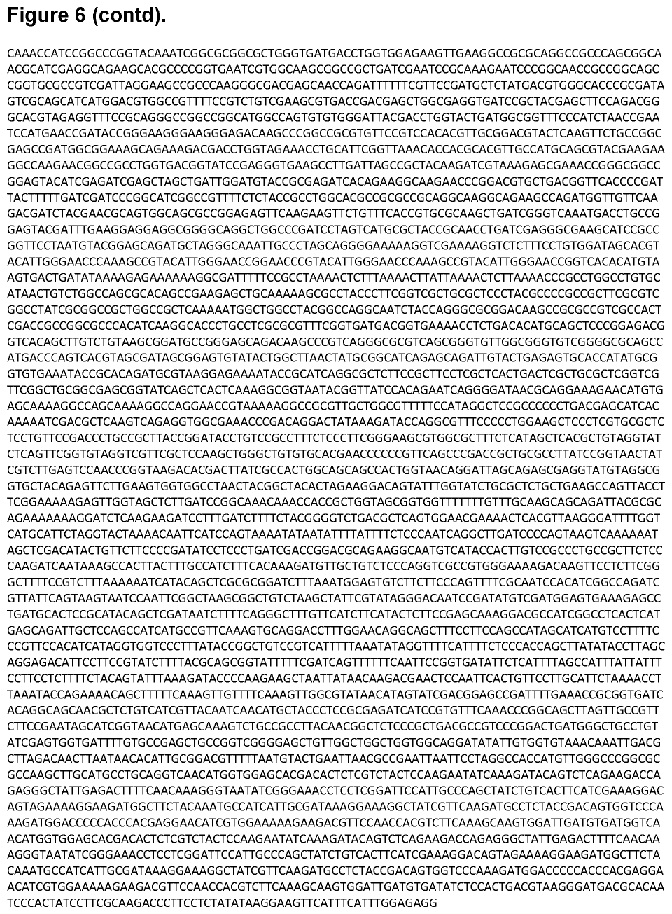

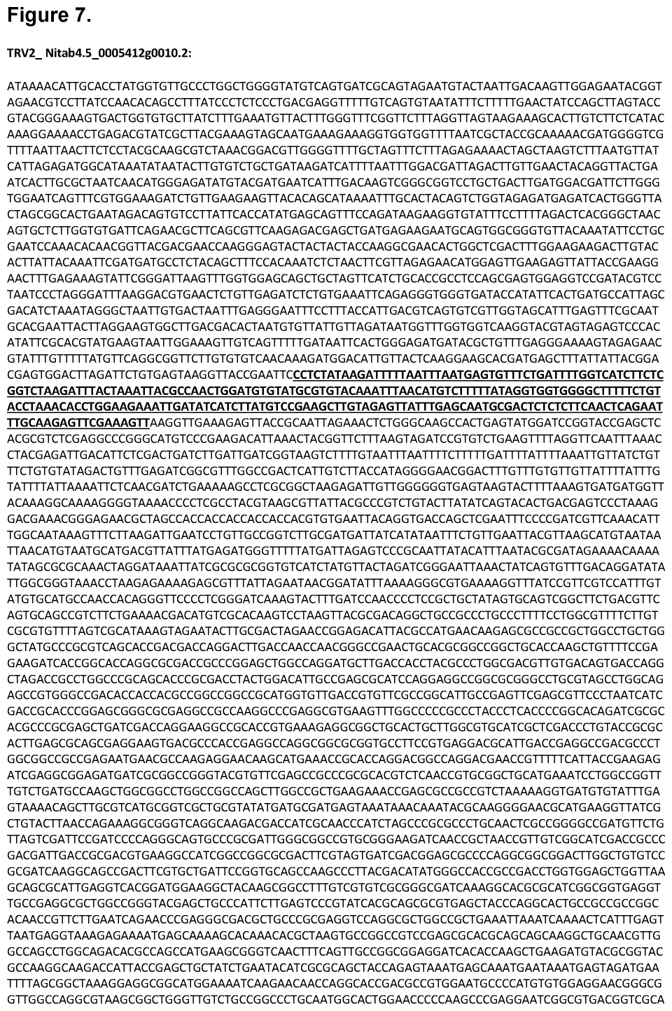

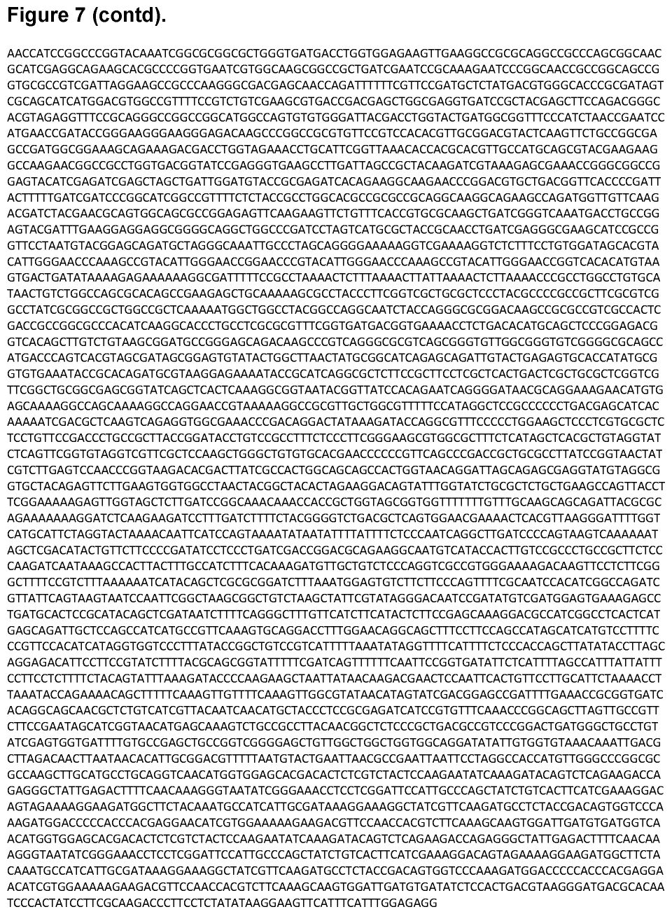

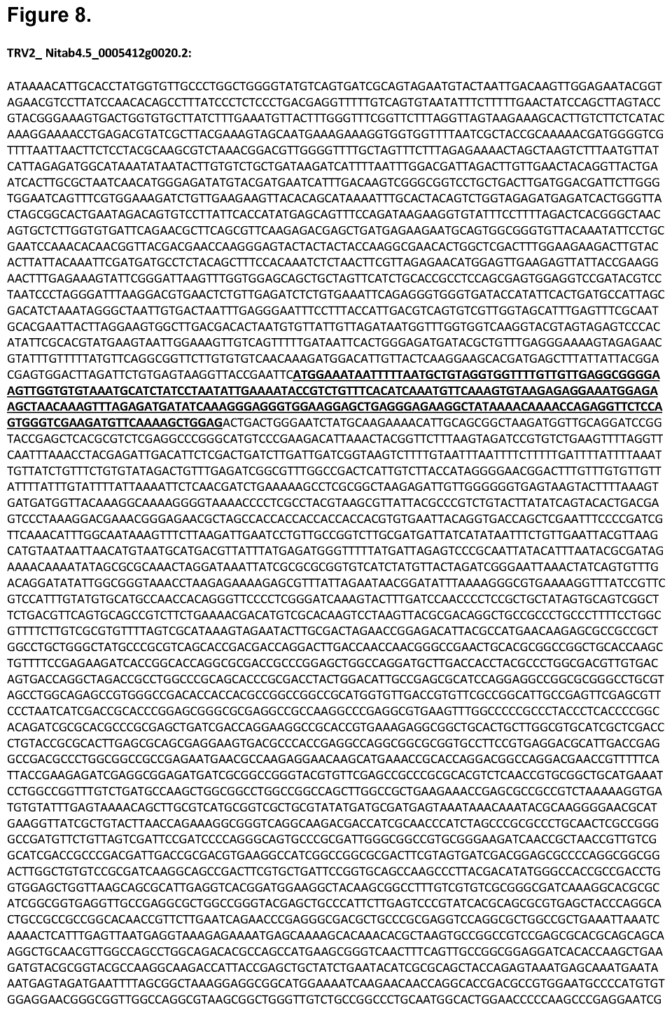

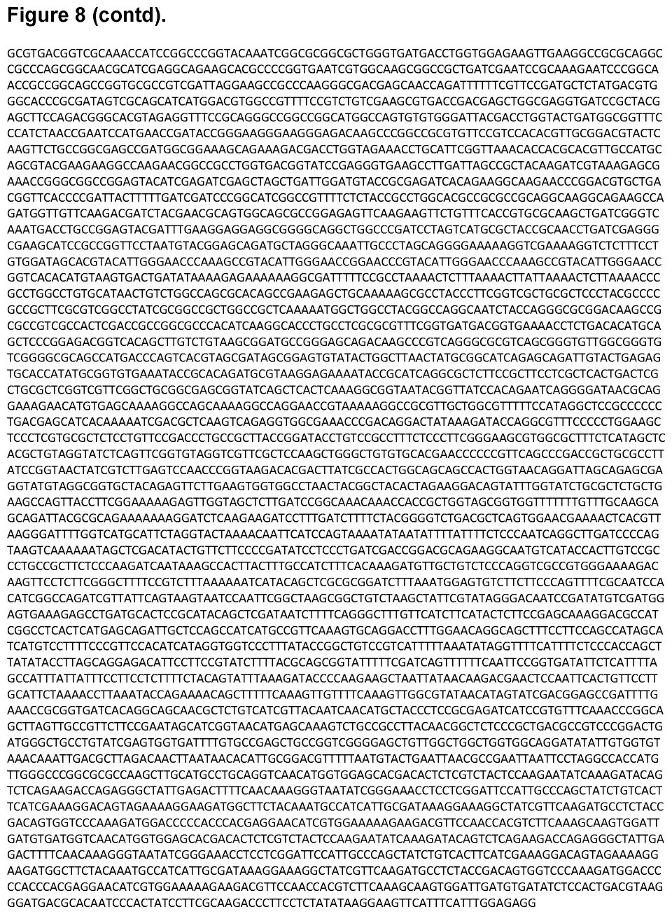

The TRV RNA2 sequences are shown in FIGS. 5-8 in which the gene-specific sequences are shown in bold and underlined.

Results

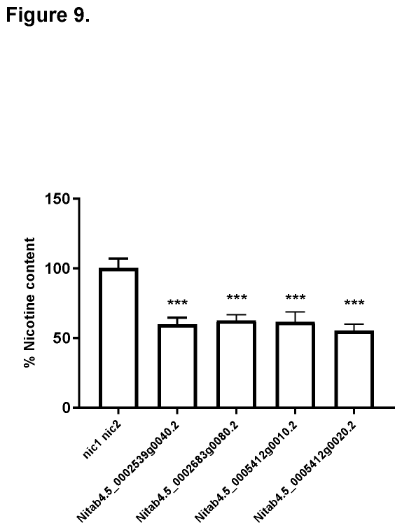

The nicotine content of 6-week-old LaBY21 (nic1nic2) leaves expressing silencing constructs for the indicated genes are shown in FIG. 9. Content is represented relative to control and comprises three biological replicates analysed by One way ANOVA. Values are shown as means±SEM. Asterisks indicate statistical significance of P value≤0.001

Silencing of genes in the Nic3 locus lead to a decrease in nicotine content as compared to nic1 nic2.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

Adams, A. C., De Godoy Lusso, M. S., Pramod, S., and Xu, D. (2016). Compositions and Methods for Producing Tobacco Plants and Products Having Altered Alkaloid Levels. US patent application 20160374387A1.
Bindler, G., Plieske, J., Bakaher, N., Gunduz, I., Ivanov, N., Hoeven, R., Ganal, M., and Donini, P. (2011). A high density genetic map of tobacco (Nicotiana tabacum L.) obtained from large scale microsatellite marker development. Theoretical and Applied Genetics 123, 219-230.
Collins, G. B., Legg, P. D., and Kasperba. M j (1974). Use of Anther-Derived Haploids in Nicotiana 0.1. Isolation of breeding lines differing in total alkaloid content. Crop Science 14, 77-80.
Chakrabarty, R., Banerjee, R., Chung, S. M., Farman, M., Citovsky, V., Hogenhout, S. A., Tzfira, T., and Goodin, M. (2007). PSITE vectors for stable integration or transient expression of autofluorescent protein fusions in plants: probing Nicotiana benthamiana-virus interactions. Molecular Plant-Microbe Interactions 20, 740-50.
Edwards, K. D., Fernandez-Pozo, N., Drake-Stowe, K., Humphry M., Evans, A. D., Bombarely, A., Allen, F., Hurst, R., White, B., Kernodle, S. P., Bromley, J. R., Sanchez-Tamburrino, J. P., Lewis, R. S., and Mueller, L. A. (2017) A reference genome for Nicotiana tabacum enables map-based cloning of homologous loci implicated in nitrogen utilization efficiency. BMC Genomics 18, 448.
Hibi, N., Higashiguchi, S., Hashimoto, T., and Yamada, Y. (1994). Gene-Expression in Tobacco Low-Nicotine Mutants. Plant Cell 6, 723-735.
Horsch, R. B., Fry, J. E., Hoffmann, N. L., Eichholtz, D., Rogers, S. G., and Fraley, R. T. (1985). A simple and general method for transferring genes into plants. Science 227, 1229-1231.
Kajikawa, M., Sierro, N., Kawaguchi, H., Bakaher, N., Ivanov, N. I., Hashimoto, T., and Shoji, T. (2017). Genomic insights into the evolution of the nicotine biosynthesis pathway in tobacco. Plant Physiology 174, 999-1011.
Kidd, S. K., Melillo, A. A., Lu, R. H., Reed, D. G., Kuno, N., Uchida, K., Furuya, M., and Jelesko, J. G. (2006). The A and B loci in tobacco regulate a network of stress response genes, few of which are associated with nicotine biosynthesis. Plant Mol Biol 60, 699-716.
Langmead, B., and Salzberg, S. L. (2012). Fast gapped-read alignment with Bowtie 2. Nat Methods 9, 357-9.
Legg P, Chaplin J, Collins G. (1969). Inheritance of percent total alkaloids in Nicotiana tabacum L.: populations derived from crosses of low alkaloid lines with burley and flue-cured varieties. Journal of Heredity 60: 213-217.
Legg P., and G., C. (1971). Inheritance of percent total alkaloids in Nicotiana tabacum L. II. genetic effects of two loci in Burley21×LA Burley 21 populations. Canadian Journal of Genetics and Cytology 13, 287-291.
Legg P D, Collins G B, Litton C C. 1970. Registration of La Burley-21 Tobacco Germplasm. Crop Science 10(2): 212.
McKenna, A., Hanna, M., Banks, E., Sivachenko, A., Cibulskis, K., Kernytsky, A., Garimella, K., Altshuler, D., Gabriel, S., Daly, M., and DePristo, M. A. (2010). The Genome Analysis Toolkit: a MapReduce framework for analyzing next-generation DNA sequencing data. Genome Research 20, 1297-303.
Neff, M. M., Turk, E., and Kalishman, M. (2002). Web-based primer design for single nucleotide polymorphism analysis. Trends in Genetics 18, 613-5.
Nielsen, M. T., Legg, P. D., and Collins, G. B. (1988). Registration of HI and LI burley 21 tobacco germplasms. Crop Science 28, 206-207.
Qin, Q., Li, D., Dai, X., Zhao, P., Miller, R., Jack, A., and Yang, S. (2015) Development of user-friendly marker for Nic2 in tobacco. SRC, Tobacco Science Research Conference, 69, abstract 79.
Reed, D. G., and Jelesko, J. G. (2004). The A and B loci of Nicotiana tabacum have non-equivalent effects on the mRNA levels of four alkaloid biosynthetic genes. Plant Science 167 1123-1130.
Rigola, D., van Oeveren, J., Janssen, A., Bonné, A., Schneiders, H., van der Poel, H. J. A., van Orsouw, N. J., Hogers, R. C. J., de Both, M. T. J., and van Eijk, M. J. T. (2009) High-Throughput Detection of Induced Mutations and Natural Variation Using KeyPoint™ Technology. PLOS ONE 4, e4761. https://doi.org/10.1371/journal.pone.0004761.
Rushton P J, Bokowiec M T, Han S C, Zhang H B, Brannock J F, Chen X F, Laudeman T W, Timko M P. 2008. Tobacco transcription factors: Novel insights into transcriptional regulation in the Solanaceae. Plant Physiology 147: 280-295.
Sahoo, D. K., Dey, N., and Maiti, I. B. (2014). pSiM24 is a novel versatile gene expression vector for transient assays as well as stable expression of foreign genes in plants. PLoS One 9, e98988. Shoji, T., Kajikawa, M., and Hashimoto, T. (2010). Clustered transcription factor genes regulate nicotine biosynthesis in tobacco. The Plant Cell 22,3390-3409.
Sierro, N., Battey, J. N., Ouadi, S., Bakaher, N., Bovet, L., Willig, A., Goepfert, S., Peitsch, M. C., and Ivanov, N. V. (2014). The tobacco genome sequence and its comparison with those of tomato and potato. Nature Communications 5, 3833.
Stam, P. (1993). Construction of integrated genetic linkage maps by means of a new computer package—Joinmap. Plant Journal 3, 739-744.
Valleau W. 1949. Breeding low-nicotine tobacco. Journal of Agricultural Research 78: 171-181. Van Ooijen J. W. (2009). MapQTL 6: Software for the mapping of quantitative trait loci in experimental populations of diploid species. Wageningen (The Netherlands): Kyazma B V.
Voelckel, C., Krugel, T., Gase, K., Heidrich, N., van Dam, N. M., Winz, R., and Baldwin, I. T. (2001). Anti-sense expression of putrescine N-methyltransferase confirms defensive role of nicotine in Nicotiana sylvestris against Manduca sexta. Chemoecology 11, 121-126.
Zhu, B., Cai, G., Hall, E. O., and Freeman, G. J. (2007). In-fusion assembly: seamless engineering of multidomain fusion proteins, modular vectors, and mutations. Biotechniques 43, 354-9.

1. A method of modulating (e.g. decreasing) the alkaloid content (e.g. nicotine content) of a tobacco plant or a part thereof, or tobacco plant cell, the method comprising modifying said plant or cell by modulating the activity or expression of:
a) at least one Nic3 gene;
and optionally
b) at least one Nic1 ERF gene; and/or
c) at least one Nic2 ERF gene.

2. A method of modulating (e.g. decreasing) the alkaloid content (e.g. nicotine content) of a tobacco plant or a part thereof, or tobacco plant cell, the method comprising modifying said plant or part thereof, or cell by: introducing at least one mutation to a Nic3 locus (e.g. in a Nic3 gene), and optionally at least one mutation to a Nic1 locus (e.g. in a Nic1 ERF gene) and/or at least one mutation to a Nic2 locus (e.g. in a Nic2 ERF gene).

3. A method of modulating (e.g. decreasing) the content of a tobacco specific nitrosamine (TSNA) precursor in a tobacco plant or plant part thereof, or tobacco plant cell, the method comprising modifying said plant or cell by:
i) modulating the activity or expression of:
a) at least one Nic3 gene; and optionally
b) at least one Nic1 ERF gene; and/or
c) at least one Nic2 ERF gene; or
ii) introducing at least one mutation to a Nic3 locus (e.g. in a Nic3 gene), and optionally at least one mutation to a Nic1 locus (e.g. in a Nic1 ERF gene) and/or at least one mutation to a Nic2 locus (e.g. in a Nic2 ERF gene).

4. Use of:
a) at least one Nic3 gene, and optionally at least one Nic1 ERF gene and/or at least one Nic2 ERF gene; or
b) at least one mutation in a Nic3 locus (e.g. in a Nic3 gene), and optionally at least one mutation in a Nic1 locus (e.g. in a Nic1 ERF gene) and/or at least one mutation in a Nic2 locus (e.g. in a Nic2 ERF gene);
for modulating (e.g. decreasing) alkaloid content (e.g. nicotine content) and or TSNA precursor content of a tobacco plant or part thereof or tobacco plant cell.

5. A method for producing a plant or part thereof, a tobacco plant cell, a tobacco plant propagation material, a tobacco leaf, a cut harvested tobacco leaf, a processed tobacco leaf or a cut and processed tobacco leaf which has modulated (e.g. decreased) alkaloid content (e.g. nicotine content), the method comprising modifying said tobacco plant or part thereof or tobacco cell to:
i) modulate the activity or expression of:
a) at least one Nic3 gene; and optionally
b) at least one Nic1 ERF gene; and/or
c) at least one Nic2 ERF gene; or
ii) introduce at least one mutation in a Nic3 locus (e.g. in a Nic3 gene), and optionally at least one mutation in a Nic1 locus (e.g. in a Nic1 ERF gene) and/or at least one mutation in a Nic2 locus (e.g. in a Nic2 ERF gene).

6. A method or use according to any one of the preceding claims, wherein the nicotine content is decreased in comparison to a tobacco plant or part thereof or tobacco cell which has not been modified to introduce at least one mutation to a Nic3 locus, and optionally at least one mutation to a Nic1 locus and/or at least one mutation to a Nic2 locus.

7. A tobacco plant or part thereof or tobacco cell which has been modified to achieve a reduction in alkaloid content (e.g. nicotine content) in comparison to an unmodified tobacco plant or part thereof or tobacco cell, wherein said modification comprises:
i) modulated activity of or expression of:
a) at least one Nic3 gene; and optionally
b) at least one Nic1 ERF gene; and/or
c) at least one Nic2 ERF gene; or
ii) at least one mutation in a Nic3 locus (e.g. in a Nic3 gene), and optionally at least one mutation in a Nic1 locus (e.g. in a Nic1 ERF gene) and/or at least one mutation in a Nic2 locus (e.g. in a Nic2 ERF gene).

8. A tobacco plant propagation material obtainable from a tobacco plant or part thereof or tobacco cell according to claim 7 or from a tobacco plant or part thereof or tobacco cell produced by the method of any one of claim 5 or 6.

9. A method or use according to any one of claims 1-6, a plant or part thereof or cell according to claim 7, or a plant propagation material according to claim 8 wherein:
a) the activity or expression of a Nic3 gene selected from SEQ ID No. 73, 118, 124 or 127 or a sequence which has at least 90% identity thereto or a functional variant or functional fragment or orthologue of said gene is modulated; or said at least one mutation in the Nic3 locus is in a Nic3 gene selected from SEQ ID No. 73, 118, 124 or 127 or a sequence which has at least 90% identity thereto or a functional variant or functional fragment or orthologue of said gene:
b) the activity or expression of a Nic1 ERF gene selected from those listed in Table 1 is modulated; or said at least one mutation in the Nic1 locus is in a Nic1 ERF gene selected from:
SEQ ID No. 8; or SEQ ID No. 12; or SEQ ID No. 16; or SEQ ID No. 20; or SEQ ID No. 24; or SEQ ID No. 28; or SEQ ID No. 32; or a sequence which has at least 90% identity thereto, or a functional variant or functional fragment or orthologue of said gene; and/or
c) the activity or expression of a Nic2 ERF gene selected from those listed in Table 2 is modulated; or said at least one mutation in the Nic2 locus is in a Nic2 ERF gene selected from: SEQ ID No. 69; SEQ ID No. 37; or SEQ ID No. 41; or SEQ ID No. 45; or SEQ ID No. 49; or SEQ ID No. 53; or SEQ ID No. 57; or SEQ ID No. 61; or SEQ ID No. 65; or a sequence which has at least 90% identity thereto or a functional variant or functional fragment or orthologue of said gene.

10. A method or use according to any one of claim 1-6 or 9, a plant or part thereof according to claim 7 or 9, or a plant propagation material according to claim 8 or 9 wherein:
i) the activity or expression of SEQ ID No. 8 is modulated; or said at least one mutation in the Nic1 locus is in SEQ ID No. 8; and/or
ii) the activity or expression of SEQ ID No. 69 is modulated; or said at least one mutation in the Nic2 locus is in SEQ ID No. 69.

11. A method or use according to any one of claim 1-6 or 9 or 10, a plant or part thereof according to claim 7 or 9, or a plant propagation material according to claims 8 to 10 wherein said at least one mutation in the Nic3 locus is in a Nic3 gene selected from SEQ ID No. 73, 118, 124 or 127 or a sequence which has at least 90% identity thereto or a functional variant or functional fragment or orthologue of said gene and said at least one mutation in the Nic3 gene is selected from:
i) a mutation in the Nic3 gene SEQ ID No. 73 or a sequence which has at least 90% identity thereto or a functional variant or functional fragment or orthologue of said gene that results in a mutation in amino acid residues 74 to 258 or 483-538 of SEQ ID No. 75 or a sequence which has at least 90% identity thereto, or a functional variant or functional fragment or orthologue of said polypeptide;
ii) a mutation in the Nic3 gene SEQ ID No. 118 or a sequence which has at least 90% identity thereto or a functional variant or functional fragment or orthologue of said gene that results in a mutation in amino acid residues 120-584 of SEQ ID No. 120 or a sequence which has at least 90% identity thereto, or a functional variant or functional fragment or orthologue of said polypeptide;
iii) a mutation in the Nic3 gene SEQ ID No. 124 or a sequence which has at least 90% identity thereto or a functional variant or functional fragment or orthologue of said gene that results in a mutation in amino acid residues 166-406 or 483-970 of SEQ ID No. 126 or a sequence which has at least 90% identity thereto, or a functional variant or functional fragment or orthologue of said polypeptide; and
iv) a mutation in the Nic3 gene SEQ ID No. 127 or a sequence which has at least 90% identity thereto or a functional variant or functional fragment or orthologue of said gene that results in a mutation in amino acid residues 171-406 or 509-967 of SEQ ID No. 129 or a sequence which has at least 90% identity thereto, or a functional variant or functional fragment or orthologue of said polypeptide.

12. Use of a plant or part thereof according to any one of claim 7 or 9-11, or of a plant produced by the method of any one of claim 5 or 6, or 9 to 11 to breed a plant.

13. Use of a plant or part thereof according to any one of claim 7 or 9-11, or of a plant produced by the method of any one of claim 5 or 6, or 9 to 11 for production of a product.

14. Use of a plant or part thereof according to any one of claim 7 or 9-11, or of a plant produced by the method of any one of claim 5 or 6, or 9 to 11 to grow a crop.

15. Use of a plant or part thereof according to any one of claim 7 or 9-11, or of a plant produced by the method of any one of claim 5 or 6, or 9 to 11 to produce a leaf.

16. A harvested leaf of a plant according to any one of claim 7 or 9-11, or obtainable from a plant propagated from a propagation material according to any one of claims 7-11, or obtainable from a plant obtained by a use according to any one of claim 4, 6 or 9-15, or obtainable from a plant produced by the method of any one of claim 5 or 6, or 9 to 15.

17. A harvested leaf of a plant according to claim 16, wherein the harvested leaf of a plant is a cut harvested leaf.

18. A processed leaf, preferably a processed tobacco leaf, preferably a non-viable processed tobacco leaf:
obtainable (e.g. obtained) from a plant obtainable from a use according to any one of claim 4, 6 or 9-15;
obtainable (e.g. obtained) by processing a plant according to any one of claim 7 or 9-11;
obtainable (e.g. obtained) from a plant propagated from a plant propagation material according to any one of claims 8-11; or
obtainable (e.g. obtained) by processing a harvested leaf of a plant according to claim 16 or 17; or
obtainable (e.g. obtained) from a plant produced by the method of any one of claim 5 or 6, or 9 to 11.

19. A processed leaf according to claim 18, wherein the leaf is processed by curing, fermenting, pasteurising or a combination thereof, preferably wherein the content of one or more TSNAs selected from N′-nitrosonornicotine (NNN), 4-(methylnitrosamino)-1-(3-pyridyl)-1-butanone (NNK), N′-nitrosoanatabine (NAT) and N-nitrosoanabasine (NAB) is decreased, wherein preferably the content of NNN and/or NNK is modulated (e.g. decreased), wherein more preferably the content of NNN is decreased.

20. A processed leaf according to claim 18 or 19, wherein the processed leaf is a cut processed leaf.

21. Cured tobacco material made from a plant or a part thereof:
obtainable (e.g. obtained) from a plant obtainable from a use according to any one of claim 4, 6 or 9-15;
obtainable (e.g. obtained) by processing a plant according to any one of claim 7 or 9-11;
obtainable (e.g. obtained) from a plant propagated from a plant propagation material according to any one of claims 8-11; or
obtainable (e.g. obtained) by processing a harvested leaf of a plant according to claim 16 or 17; or
obtainable from a plant produced by the method of any one of claim 5 or 6, or 9 to 11.

22. A tobacco blend comprising said cured tobacco material of claim 21.

23. A delivery system prepared from:
a tobacco plant according to any one of claim 7 or 9-11, or a part thereof according to any one of claim 7 or 9-11;
a tobacco plant or part thereof propagated from a tobacco plant propagation material according to any one of claims 8-11;
a harvested leaf of a plant according to claim 16 or 17;
a processed leaf according to any one of claim 18 or 19;
or
a plant produced by the method of any one of claim 5 or 6, or 9 to 11.

24. A delivery system according to claim 23, wherein the delivery system is a combustible smoking article.

25. A delivery system according to claim 23, wherein the delivery system is a smokeless delivery system.

26. A delivery system according to claim 25, wherein the delivery system is a non-combustible aerosol provision system such as a tobacco heating device or an aerosol-generating device.

27. A combustible smoking article, non-combustible aerosol provisioning system, smokeless delivery system or tobacco heating device comprising a plant or a part thereof according to any one of claims 7-11 or an extract (e.g. a tobacco extract) thereof; or a cured tobacco material according to claim 21; or a tobacco blend according to claim 22.

28. Use of a nucleotide sequence of a Nic3 locus (e.g. a Nic3 gene selected from a gene with SEQ ID No. 73, 118, 124 or 127 or a sequence which has at least 90% identity thereto or a functional variant or functional fragment or orthologue of said gene), and optionally a Nic1 locus (e.g. a Nic1 ERF gene) and/or Nic2 locus (e.g. a Nic2 ERF gene), to select a plant having reduced alkaloid content (e.g. nicotine content) and/or reduced content of tobacco specific nitrosamine (TSNA) or a precursor of a TSNA.

29. A mutant of a plant carrying at least one heritable mutation in a Nic3 locus (e.g. in a Nic3 gene selected from a gene with SEQ ID No. 73, 118, 124 or 127 or a sequence which has at least 90% identity thereto or a functional variant or functional fragment or orthologue of said gene), and optionally at least one heritable mutation in a Nic1 locus (e.g. in a Nic1 ERF gene) and/or at least one heritable mutation in a Nic2 locus (e.g. in a Nic2 ERF gene); wherein said heritable mutations decrease the alkaloid content (e.g. nicotine content), and/or decrease the content of a tobacco specific nitrosamine (TSNA) or a precursor of a TSNA in the mutant tobacco plant relative to a comparable plant which does not carry said heritable mutations.

30. Progeny or seed of a mutant plant which carries the heritable mutation according to claim 29.

31. A harvested leaf, a processed leaf or cured tobacco material produced from a plant comprising at least one mutation in a Nic3 locus (e.g. in a Nic3 ERF gene selected from a gene with SEQ ID No. 73, 118, 124 or 127 or a sequence which has at least 90% identity thereto or a functional variant or functional fragment or orthologue of said gene), and optionally at least one mutation in a Nic1 locus (e.g. in a Nic1 ERF gene) and/or at least one mutation in a Nic2 locus (e.g. in a Nic2 ERF gene) and wherein said plant has decreased nicotine content and/or decreased content of a tobacco specific nitrosamine (TSNA) or a precursor of a TSNA relative to a comparable plant which does not carry said mutations in a Nic3 locus, and optionally a Nic1 locus and/or a Nic2 locus.
